(19) United States
(12) Patent Application Publication
(10) Pub. No.: US 2008/0125441 A1
(43) Pub. Date: May 29, 2008

(54) SPIRO AND DISPIRO 1,2,4-TRIOXOLANE ANTIMALARIALS

(75) Inventors: JONATHAN L. VENNERSTROM, Omaha, NE (US); YUXIANG DONG, Omaha, NE (US); SUSAN A. CHARMAN, Parkville (AU); SERGIO WITTLIN, Basel (CH); JACQUES CHOLLET, Basel (CH); XIAOFANG WANG, Omaha, NE (US); KAMARAJ SRIRAGHAVAN, Omaha, NE (US); LIN ZHOU, Omaha, NE (US); HUGUES MATILE, Basel (CH); WILLIAM N. CHARMAN, Parkville (AU)

Correspondence Address: MCKEE, VOORHEES & SEASE, P.L.C., 801 GRAND AVENUE, SUITE 3200, DES MOINES, IA, 50309-2721, US

(73) Assignee: MEDICINES FOR MALARIA VENTURE MMV, Geneva (CH)

(21) Appl. No.: 11/930,582

(22) Filed: Oct. 31, 2007

Related U.S. Application Data
(63) Continuation-in-part of application No. 11/121,451, filed on May 4, 2005.
Application No. 11/121,451 is a continuation-in-part of application No. 10/742,010, filed on Dec. 19, 2003, now Pat. No. 6,906,205.
Application No. 10/742,010 is a continuation-in-part of application No. PCT/US02/19767, filed on Jun. 21, 2002.

(51) Int. Cl. A61K 31/496; C07D 323/02; C07D 405/12; A61K 31/343

(52) U.S. Cl. 514/254.11; 549/339; 514/462; 544/230

(57) ABSTRACT

A means and method for treating malaria, schistosomiasis, and cancer using a Spiro or dispiro 1,2,4-trioxolane is described. The preferred 1,2,4-trioxolanes include a spiroadamantane group on one side of the trioxolane group, and a spirocyclohexyl on the other side of the trioxolane group, whereby the spirocyclohexyl ring is preferably substituted at the 4-position. In comparison to artemisinin semisynthetic derivatives, the compounds of this invention are structurally simple, easy to synthesize, non-toxic, and potent against malarial parasites.

### CROSS REFERENCE TO RELATED APPLICATION

The present invention is a continuation-in-part of U.S. Ser. No. 11/121,451, which is a continuation-in-part of U.S. Ser. No. 10/742,010 (now U.S. Pat. No. 6,906,205) filed Dec. 19, 2003, which was a continuation-in-part of PCT/US02/19767 filed Jun. 21, 2001, the disclosures of which are herein specifically incorporated by reference.

### FIELD OF THE INVENTION

This invention relates to compositions and methods for treating malaria. Specifically, this invention relates to pharmaceutical compositions including spiro and dispiro trioxolanes, and methods of their use and manufacture.

### BACKGROUND OF THE INVENTION

Malaria is an acute and often chronic infectious disease resulting from the presence of protozoan parasites within red blood cells. Caused by single-celled parasites of the genus Plasmodium, malaria is transmitted from person to person by the bite of female mosquitoes.

Although once prevalent in North America and other temperate regions of the world, today malaria occurs mostly in tropical and subtropic countries. Each year, between 400 million and 600 million people contract the disease, and 1.5 million to 2.7 million die of the disease.

Four species of Plasmodium protozoan parasites are generally responsible for malaria, including Plasmodium vivax, Plasmodium falciparum, Plasmodium malariae, and Plasmodium ovale. Of the four, Plasmodium falciparum is the most dangerous, accounting for half of all clinical cases of malaria and 90% of deaths from the disease.

The transmission of malaria begins when a female mosquito bites a human already infected with the malaria parasite. When the infected mosquito bites another human, sporozoites in the mosquito's saliva are transferred into the blood, which then travel to the liver. In the liver, the sporozoites divide rapidly, then enter the bloodstream where they invade red blood cells. Inside these blood cells, the merozoites multiply rapidly until they cause the red blood cells to burst, releasing into the blood stream a new generation of merozoites that then infect other red blood cells.

The symptoms associated with malaria are generally associated with the bursting of the red blood cells. The destruction of the red blood cells spills wastes, toxin, and other debris into the blood. This in turn causes an intense fever that can leave the infected individual exhausted and bedridden. More severe symptoms associated with repeat infections and/or infection by Plasmodium falciparum include anemia, severe headaches, convulsions, delirium and, in some instances, death.

The treatment of malaria has been especially difficult due to the ability of malaria parasites to develop resistance to drugs. Quinine, an antimalarial compound that is extracted from the bark of the South American cinchona tree, is one of the oldest and most effective pharmaceuticals in existence. The downside to quinine is that it is short-acting, and fails to prevent disease relapses. Further, quinine is associated with side effects ranging from dizziness to deafness.

Chloroquine is a synthetic chemical similar to quinine. It became the drug of choice for malaria when it was developed in the 1940s due to its effectiveness, ease of manufacture, and general lack of side effects. However, in the last few decades, malaria parasites in many areas of the world have become resistant to chloroquine.

Mefloquine is another synthetic analog of quinine that has been used in the treatment of malaria. Malaria parasites have also developed resistance to mefloquine, however. Mefloquine is also associated with undesirable central nervous side effects in some patients, including hallucinations and vivid nightmares.

Antifolate drugs are effective against malaria parasites by inhibiting their reproduction. Although the parasites have also developed a resistance to antifolate drugs, the drugs can still be used effectively in combination with other types of antimalarials. The use of combination therapies in treating malaria has the drawbacks of being inconvenient and expensive, however.

More recent developments in the treatment of malaria have involved the use of the peroxide functional group, as exemplified by the drug artemisinin, which contains a unique 1,2,4-trioxane heterocyclic pharmacophore. The antimalarial action of artemisinin is due to its reaction with the iron in free heme molecules in the malaria parasite with the generation of free radicals leading to cellular destruction.

The discovery of artemisinin (qinghaosu), a naturally occurring endoperoxide sesquiterpene lactone (Meshnick et al., 1996; Vroman et al. 1999; Dhingra et al., 2000) initiated a substantial effort to elucidate its molecular mechanism of action (Jefford, 1997; Cumming et al., 1997) and to identify novel antimalarial peroxides (Dong and Vennerstrom, 2001). Many synthetic 1,2,4-trioxanes, 1,2,4,5-tetraoxanes, and other endoperoxides have been prepared.

Although the clinically useful semisynthetic artemisinin derivatives are rapid acting and potent antimalarial drugs, they have several disadvantages including recrudescence, neurotoxicity, (Wesche et al., 1994) and metabolic instability. (White, 1994). A fair number of these compounds are quite active in vitro, but most suffer from low oral activity. (White, 1994; van Agtmael et al., 1999). Although many synthetic antimalarial 1,2,4-trioxanes have since been prepared (Cumming et al., 1996; Jefford, 1997), there exists a need in the art to identify new peroxide antimalarial agents, especially those which are easily synthesized, are devoid of neurotoxicity, and which possess improved pharmacokinetic properties, e.g. improved stability, oral absorption, etc.

### SUMMARY OF THE INVENTION

The invention describes a method and composition for treating malaria with spiro and dispiro 1,2,4-trioxolanes, their prodrugs and analogues. The invention embraces achiral, achiral diastereomers, racemic mixtures, as well as enantiomeric forms of the compounds.

The trioxolanes of this invention possess excellent potency and efficacy against Plasmodium parasites. The compounds have further been found to be effective against fascioliasis. Further, several of the trioxolanes are suitable for both oral and non-oral administration. Moreover, in comparison to artemisinin semisynthetic derivatives, the compounds of this invention are structurally simple, easy and inexpensive to synthesize, and can be used effectively alone or in conjunction with other antimalarials.

### DETAILED DESCRIPTION OF THE PREFERRED EMBODIMENT

The present invention relates to the development of Spiro and dispiro 1,2,4-trioxolanes for use in the prophylaxis and treatment of malaria. As used herein the term “prophylaxis-effective amount” refers to a concentration of compound of this invention that is effective in inhibiting, decreasing the likelihood of, or preventing infection and subsequent disease by malarial parasites. Likewise, the term “treatment-effective amount” refers to a concentration of compound that is effective in treating malaria in terms of preventing an increase in the concentration of malarial parasites, decreasing the concentration of malarial parasites, and/or “curing” a malaria infection, i.e. survival for 30 days post-infection.

In previous applications, the present inventors disclosed certain novel tetrasubstituted trioxolanes having the following structural formula:

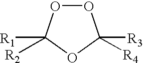

wherein R₁, R₂, R₃, and R₄ represent combinations of ring systems, acyclic systems, and functional groups that provide sufficient steric hindrance about the trioxolane ring in order to give the ring chemical and metabolic stability.

The trioxolane compounds of the present invention are iron-stable and therefore provide unexpectedly good antimalarial activity in comparison to the compounds of the inventors' prior applications.

Below are several dispiro 1,2,4-trioxolanes synthesized in accordance with the teachings of this invention. “OZ” (standing for “ozonide”) is an internal designation for these compounds that will be used throughout the remainder of the application for convenience.

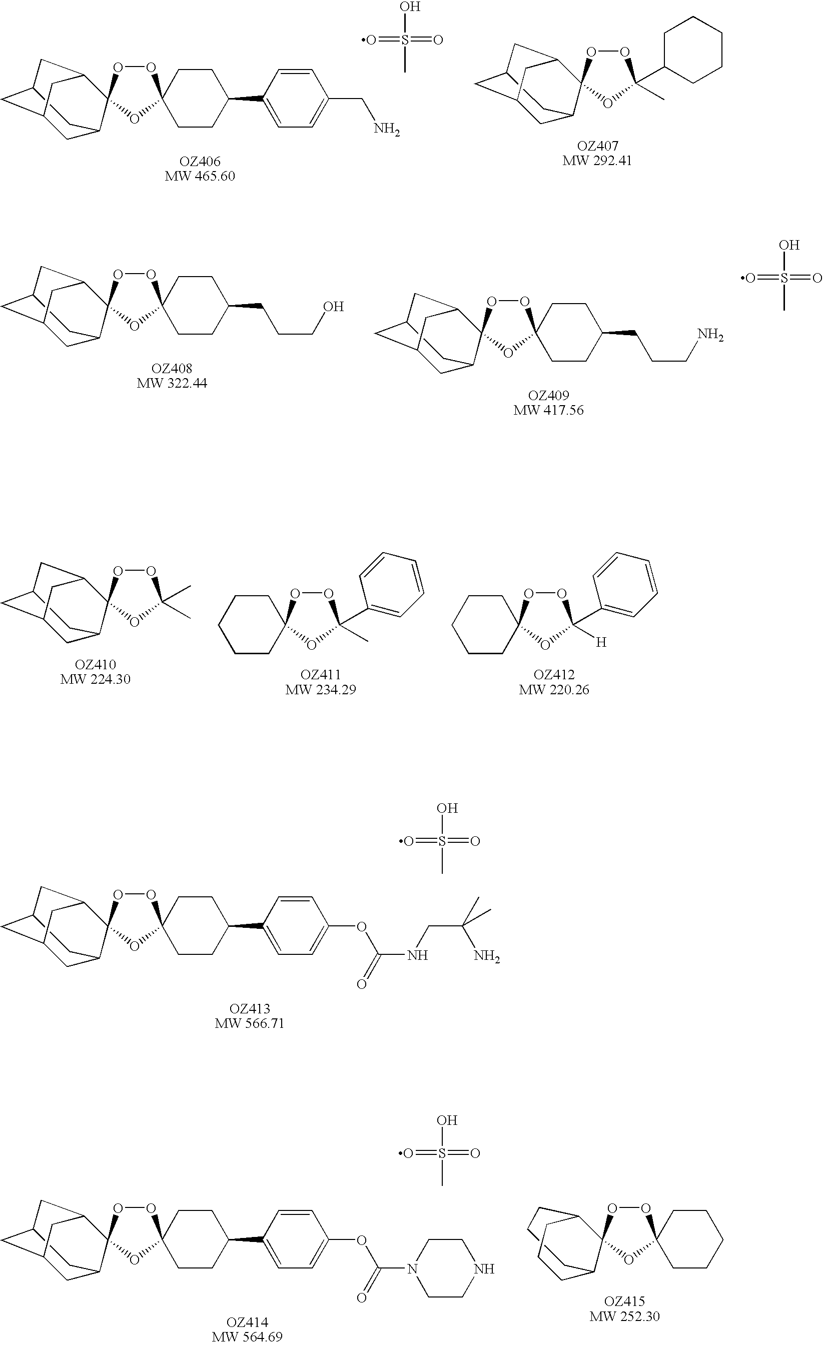

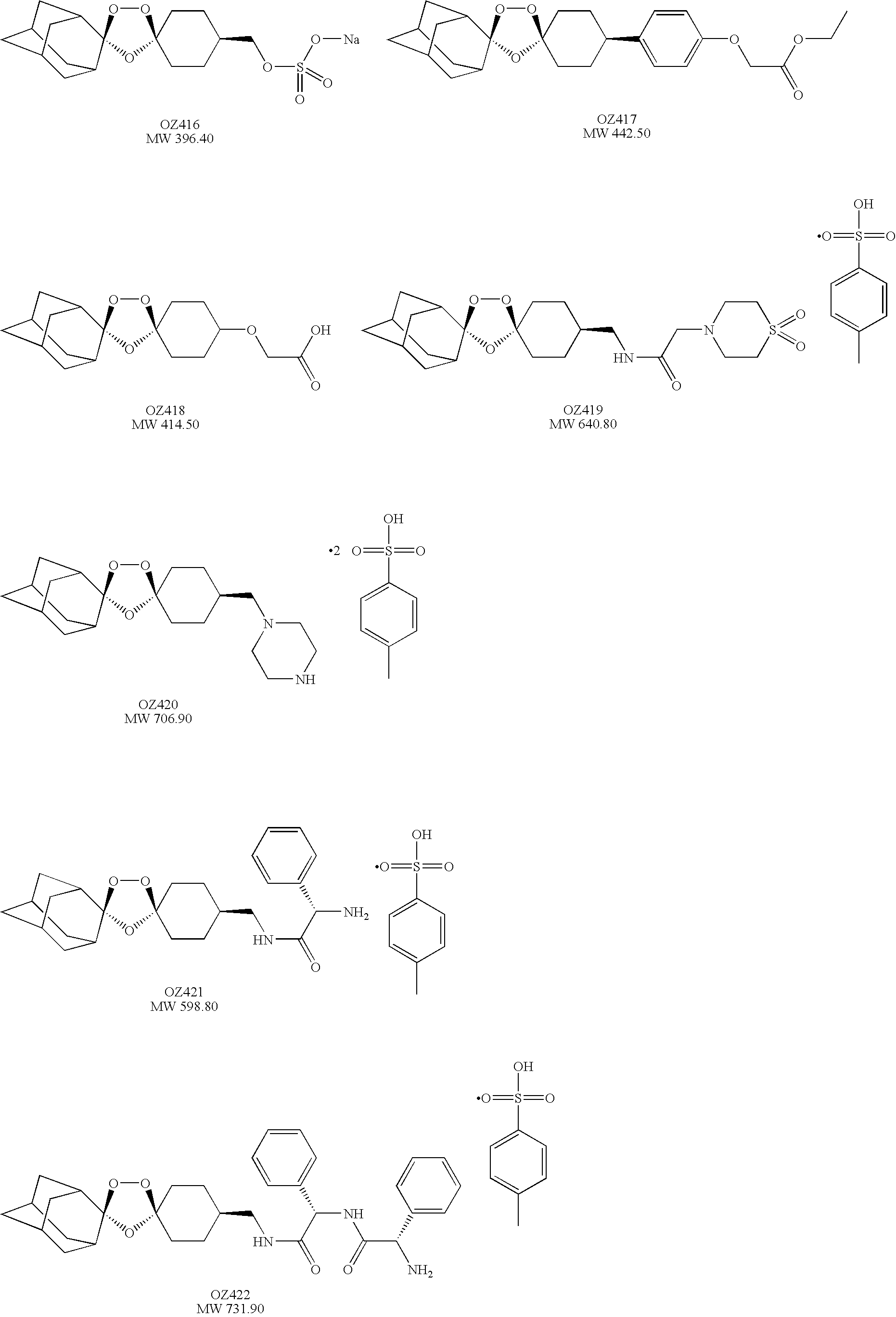

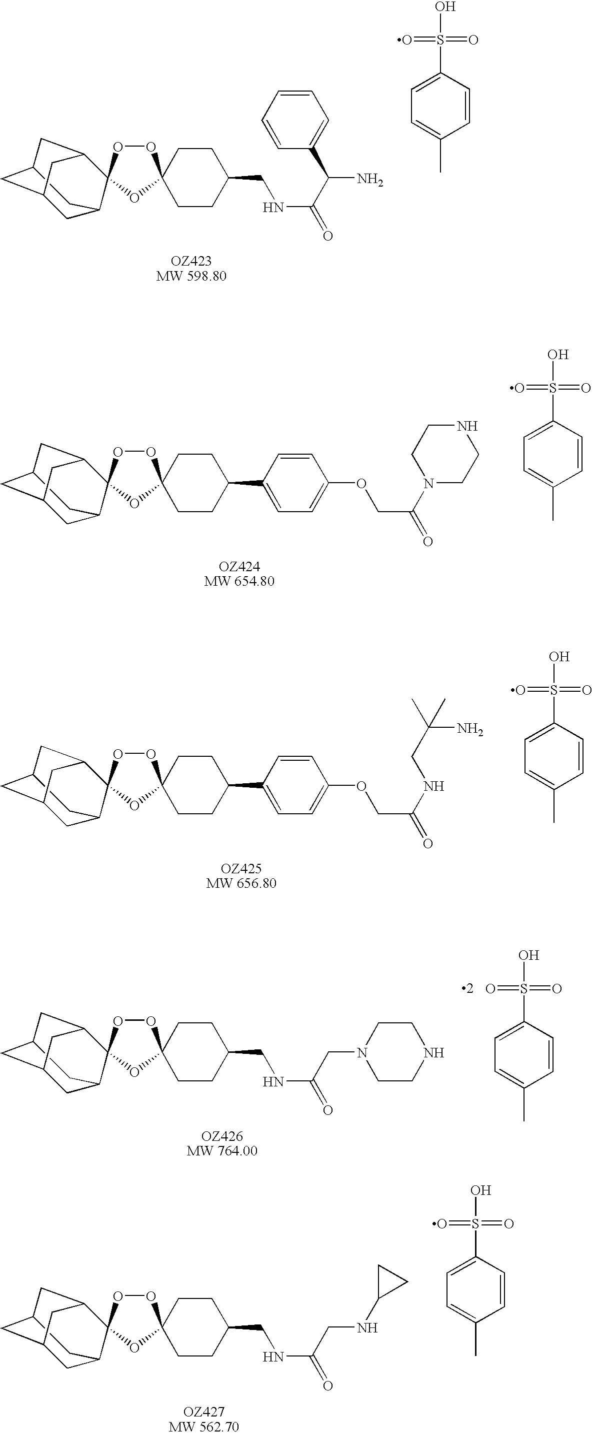

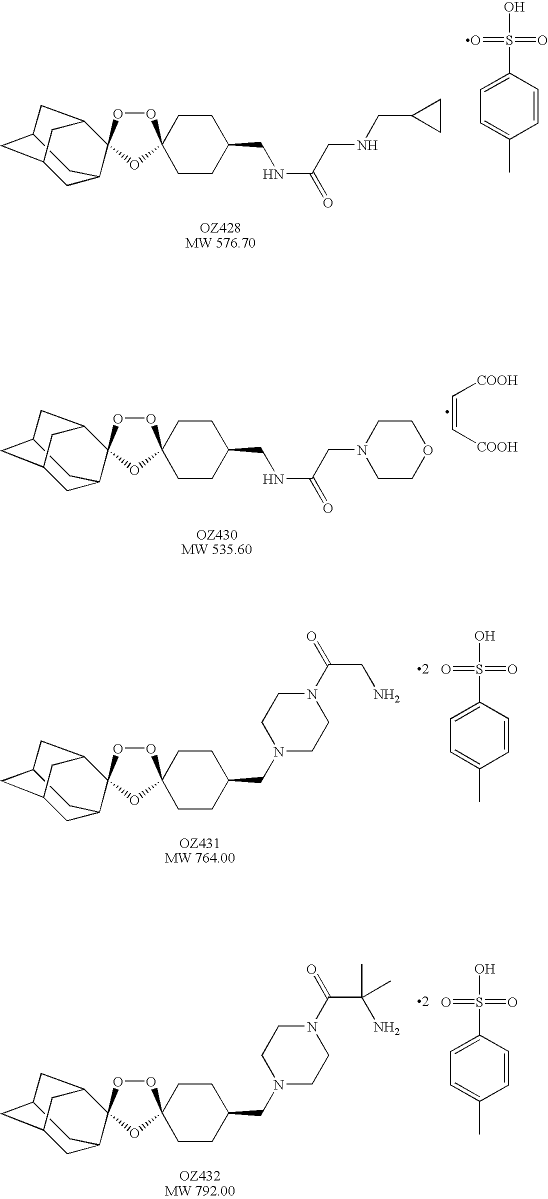

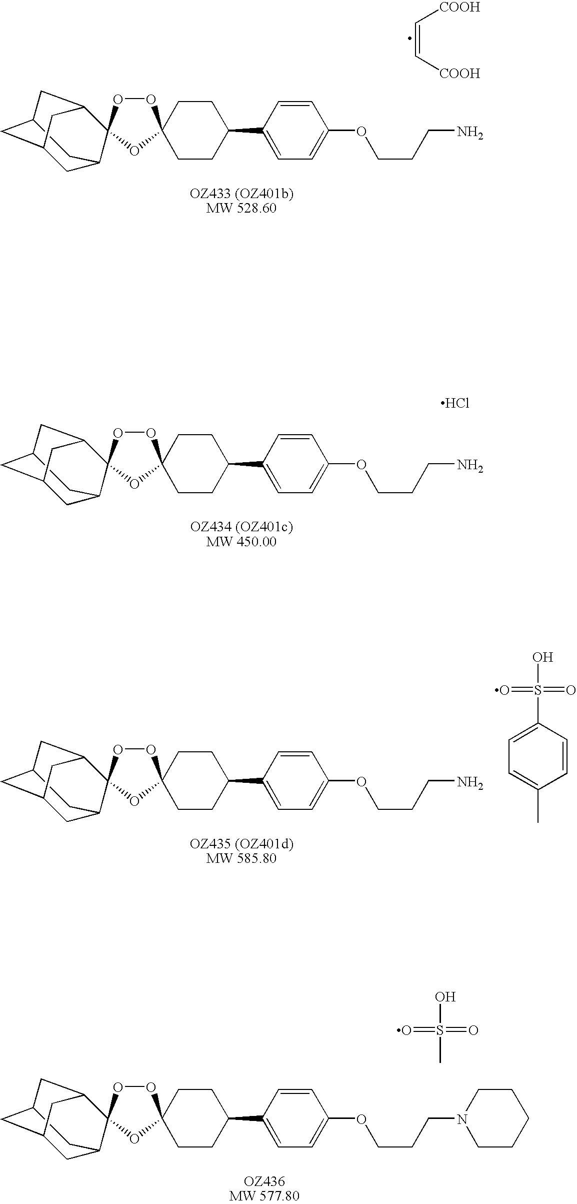

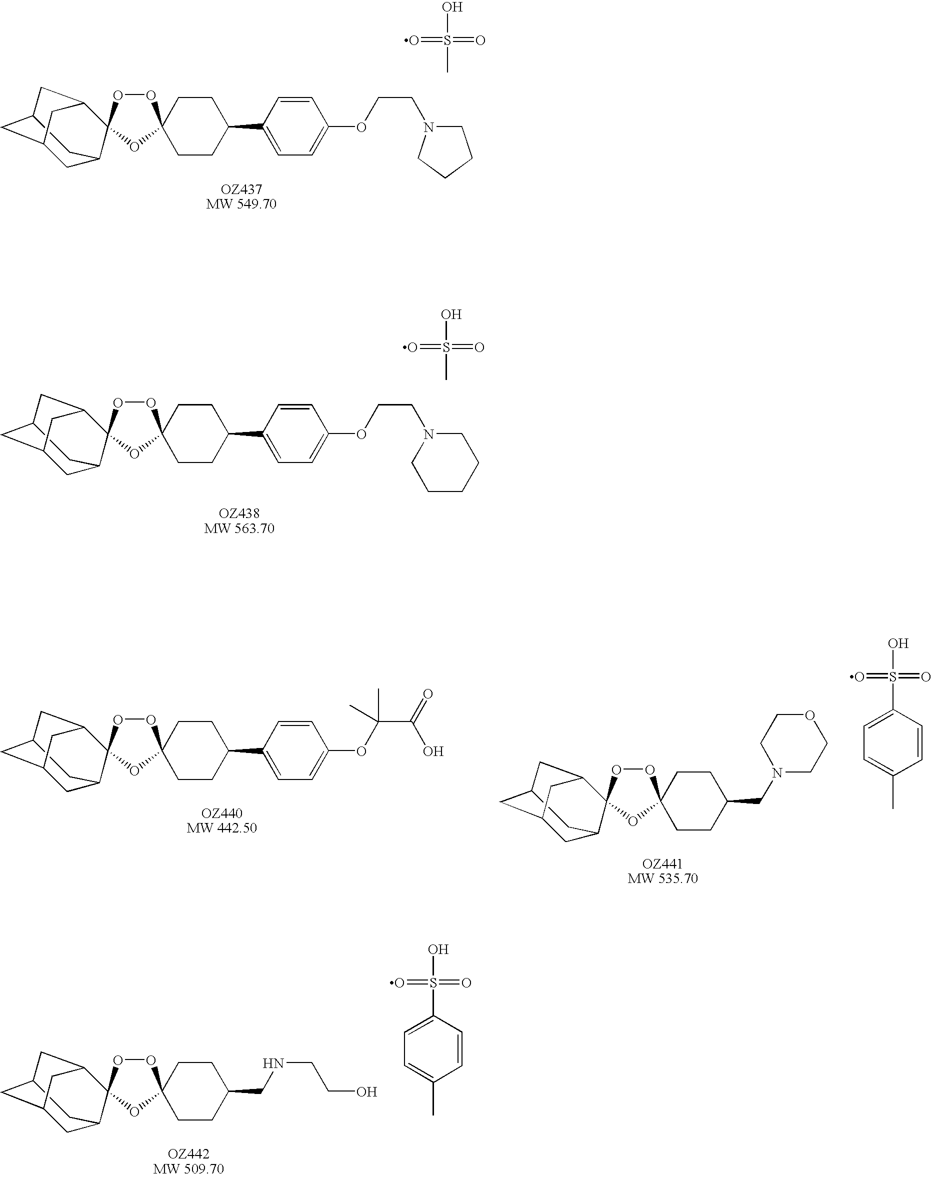

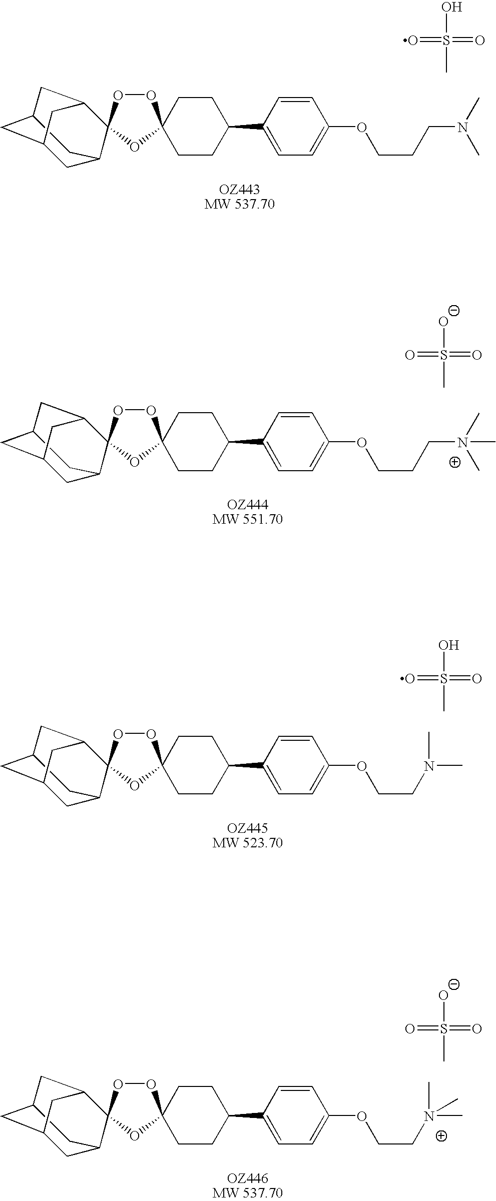

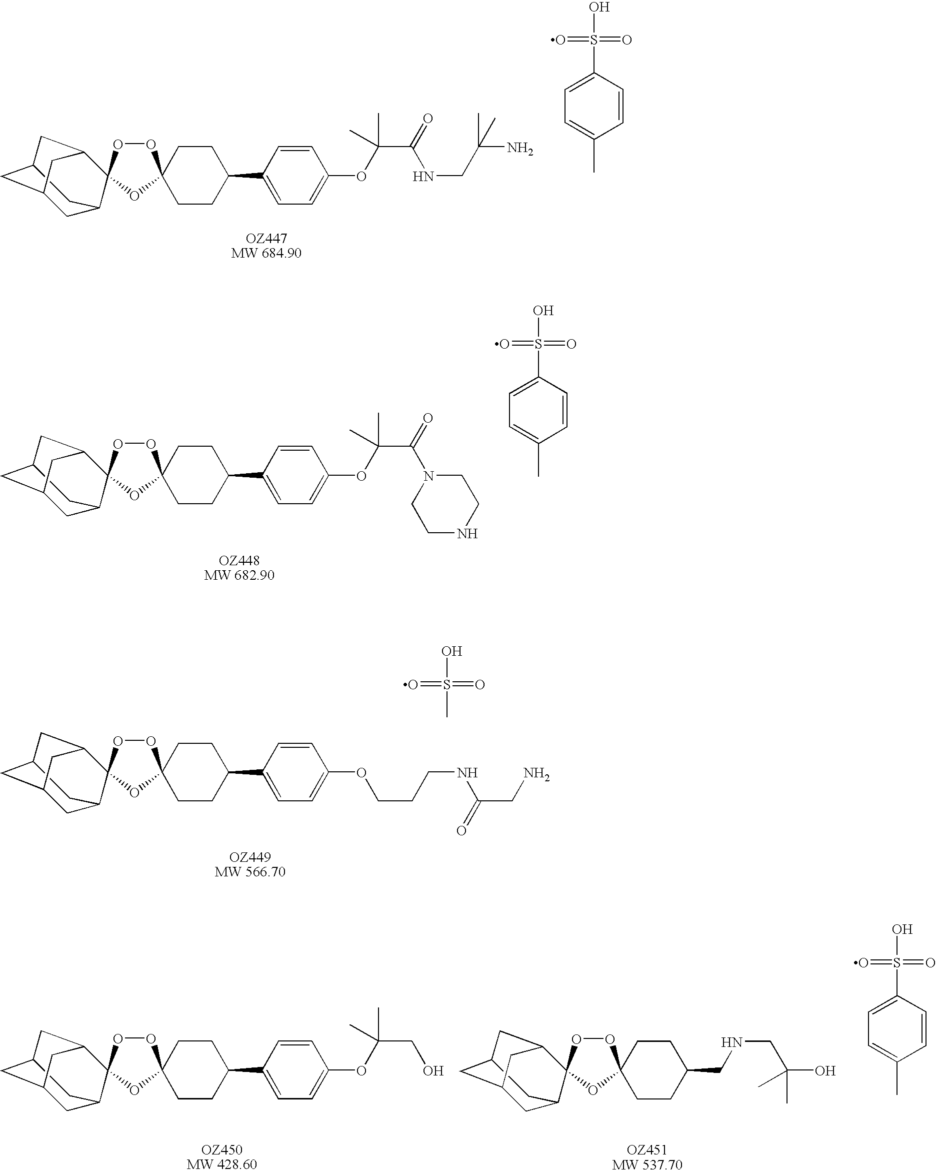

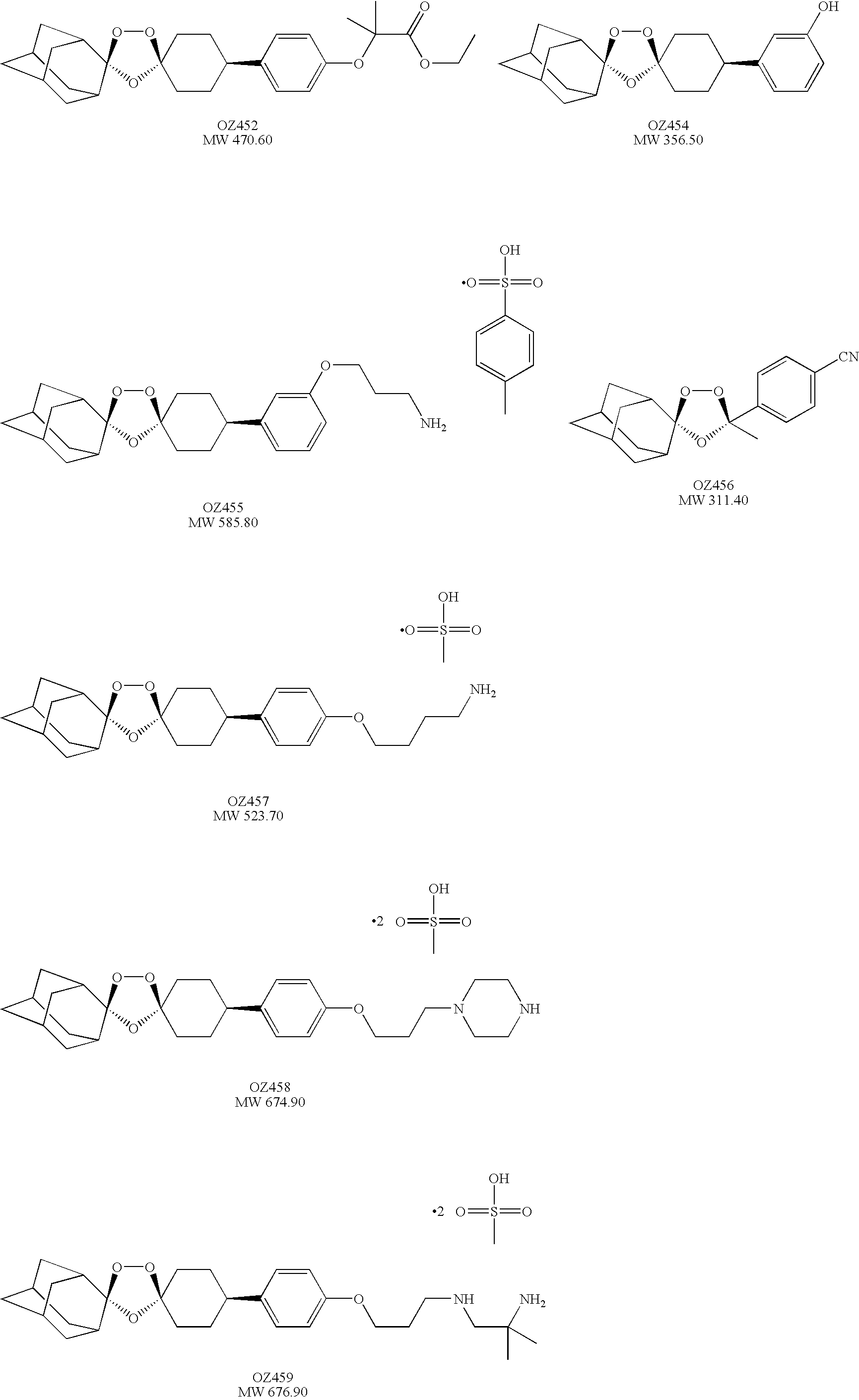

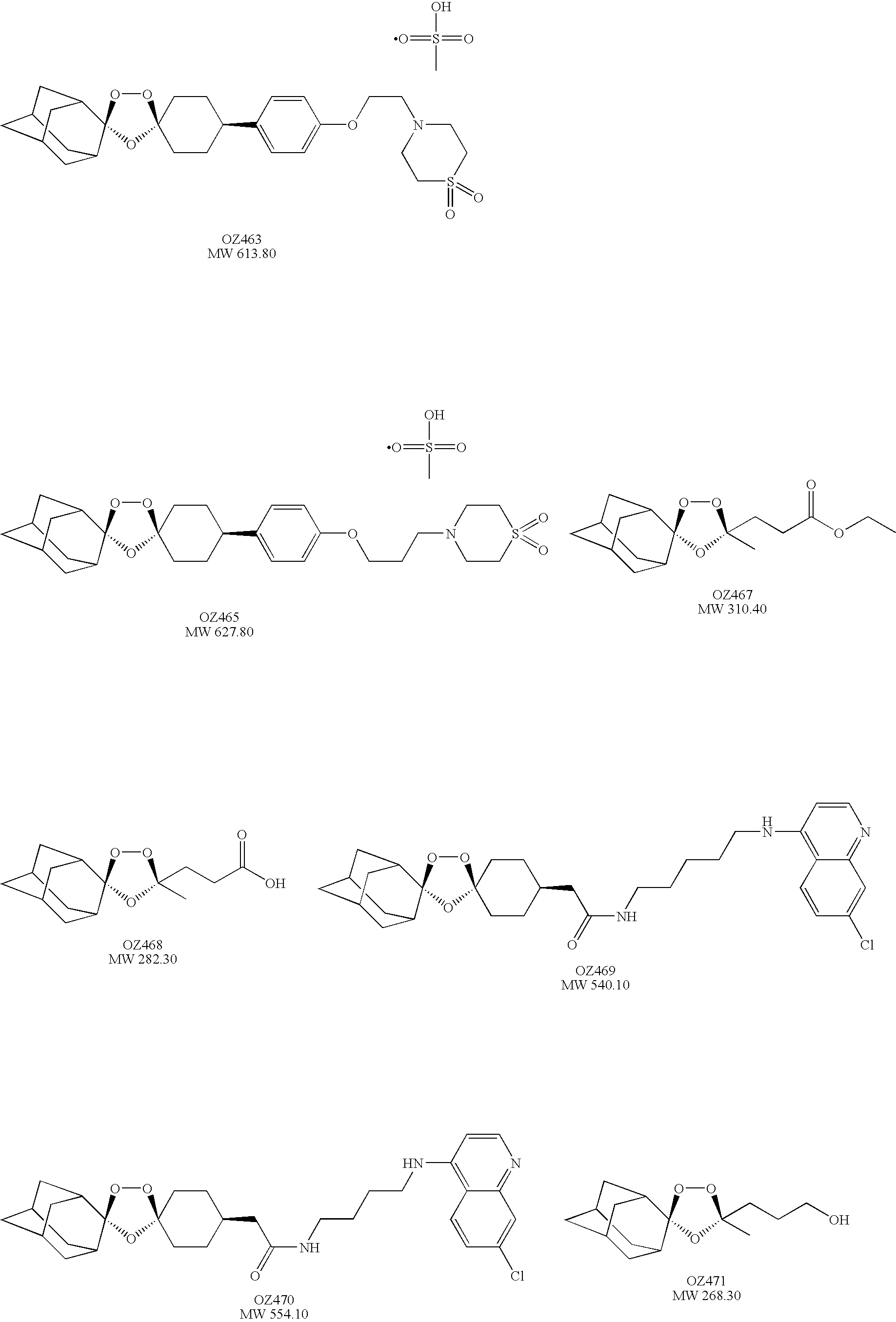

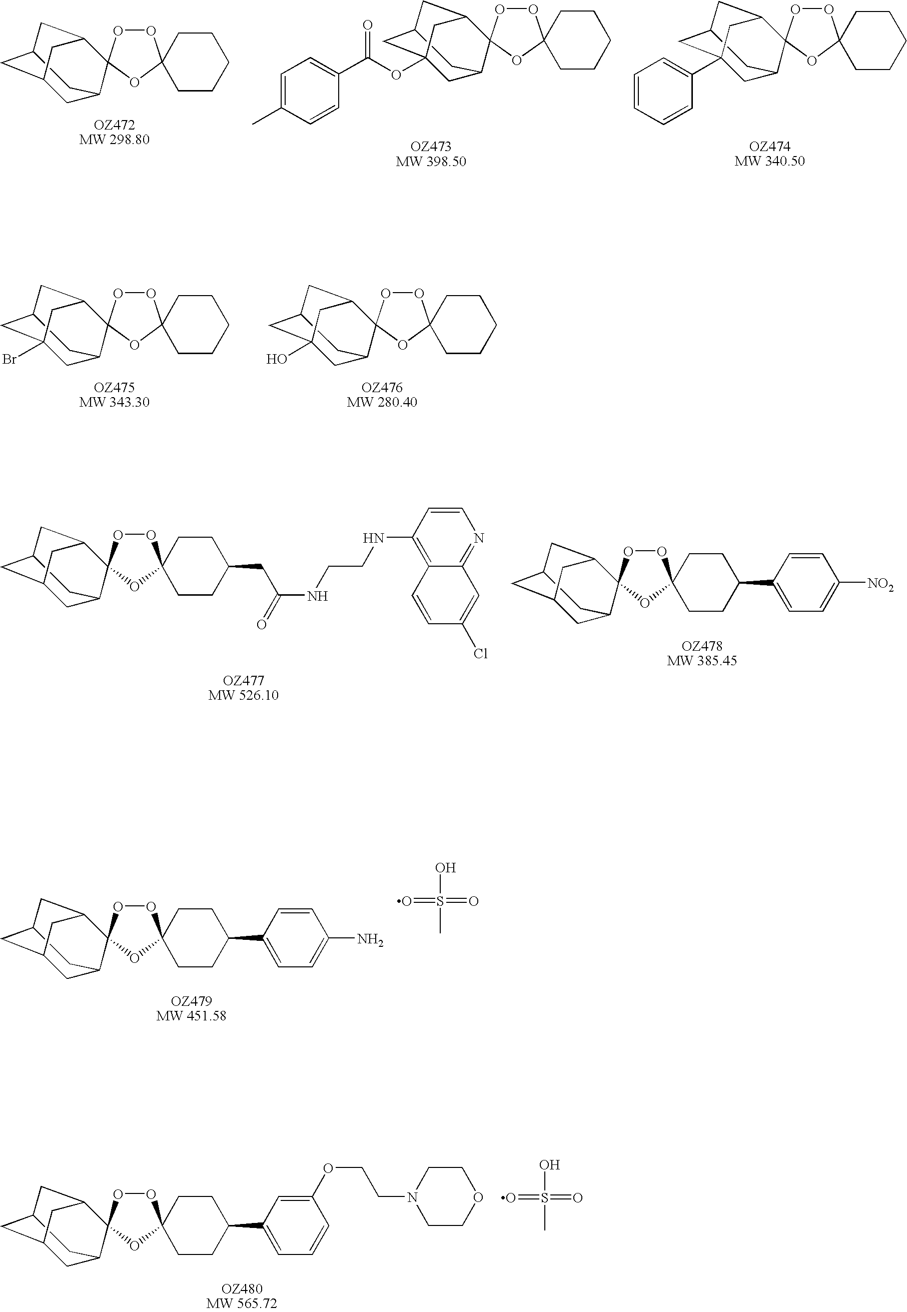

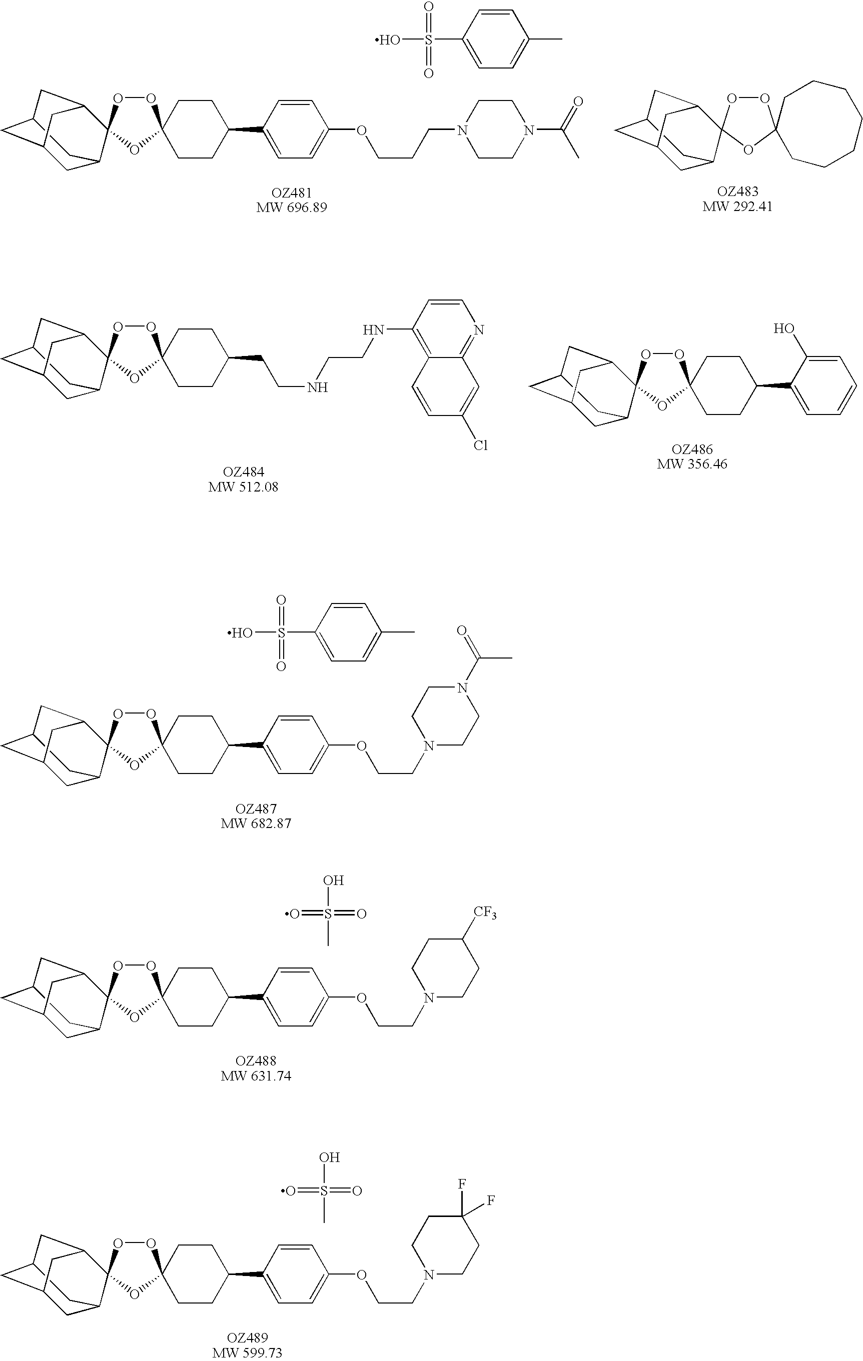

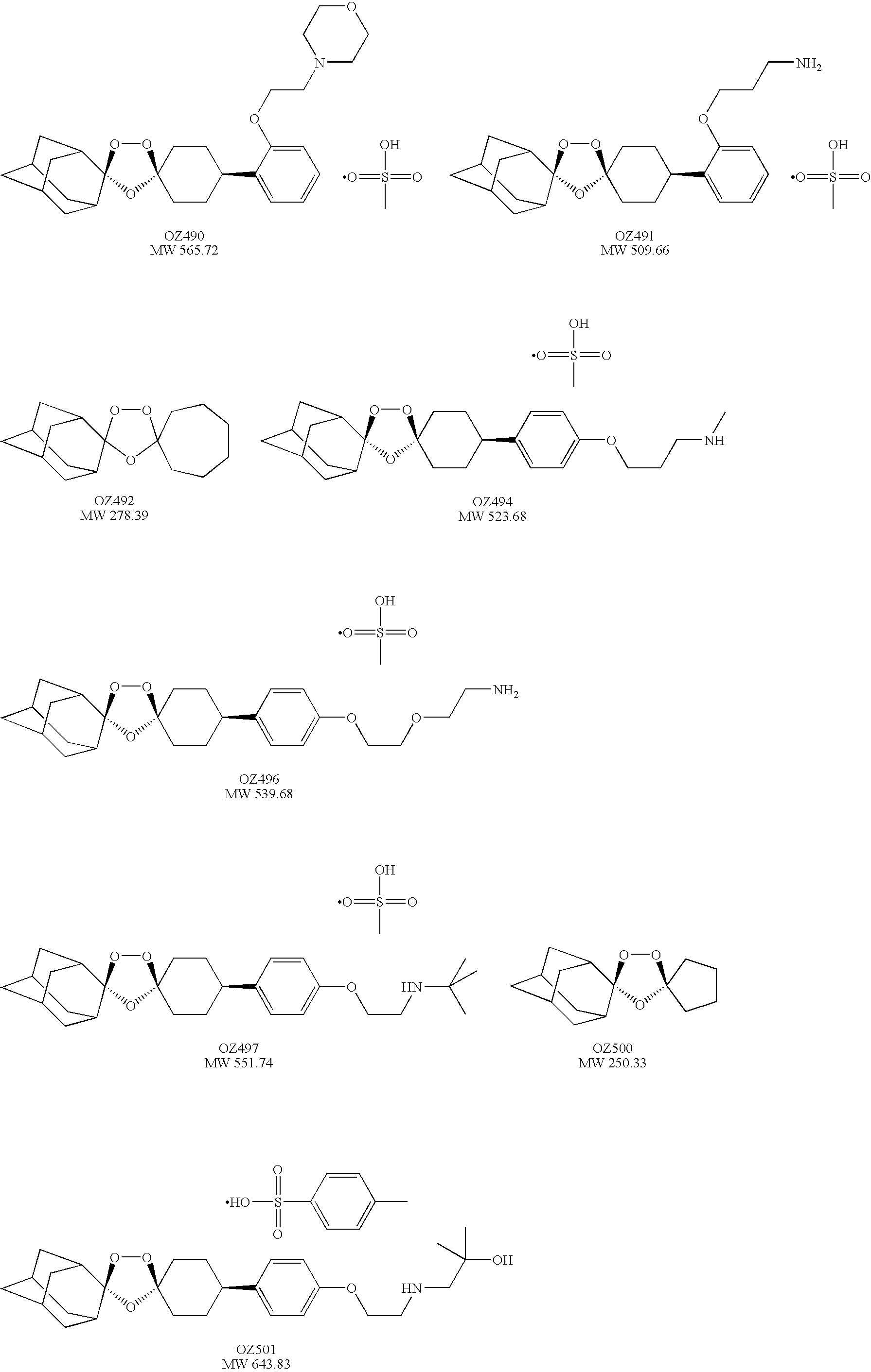

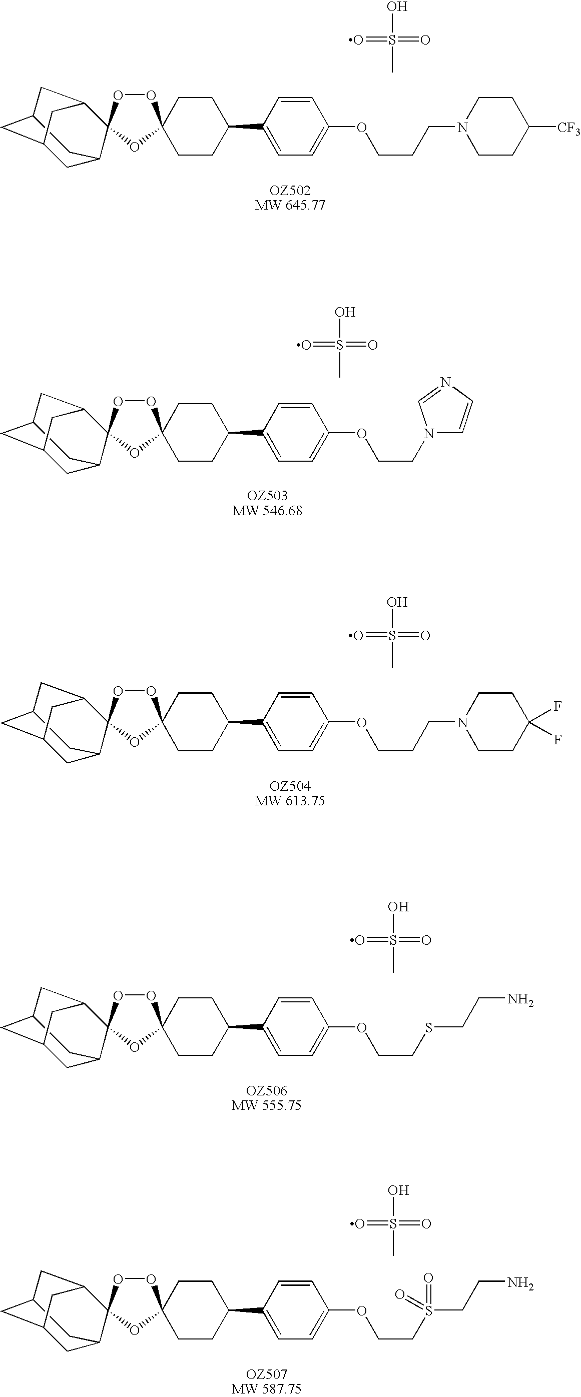

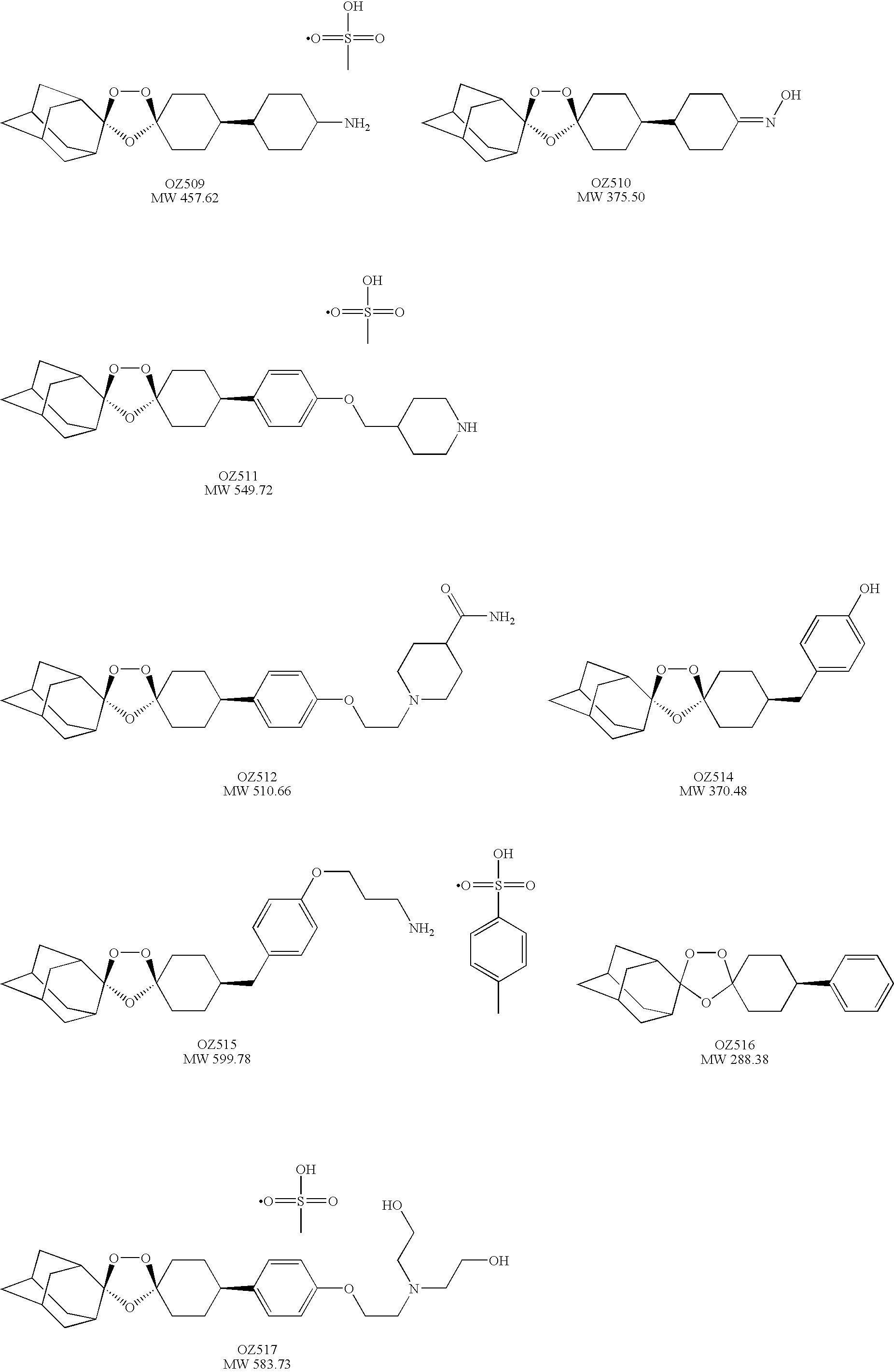

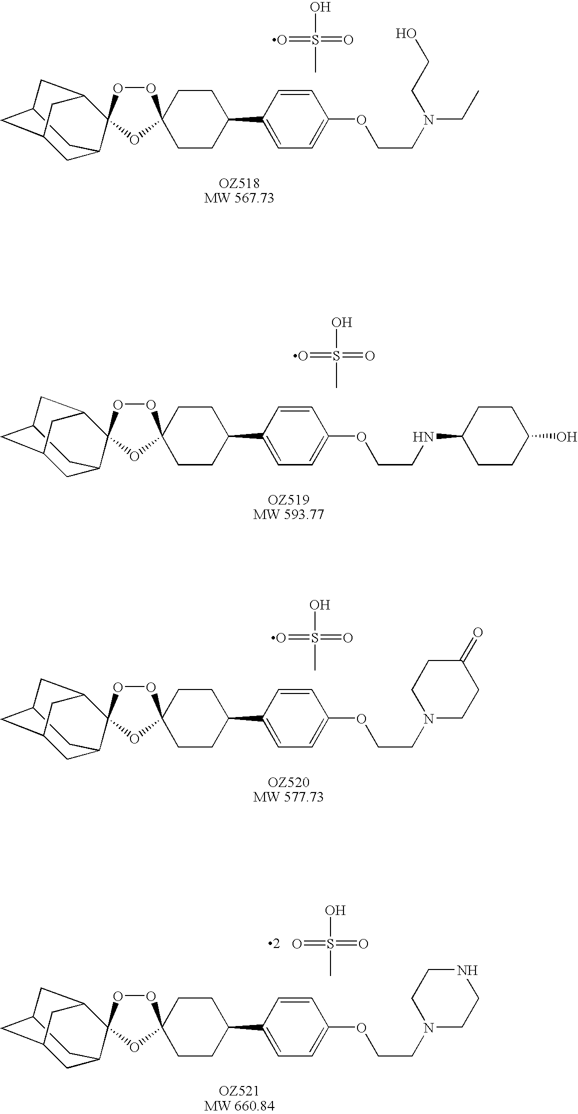

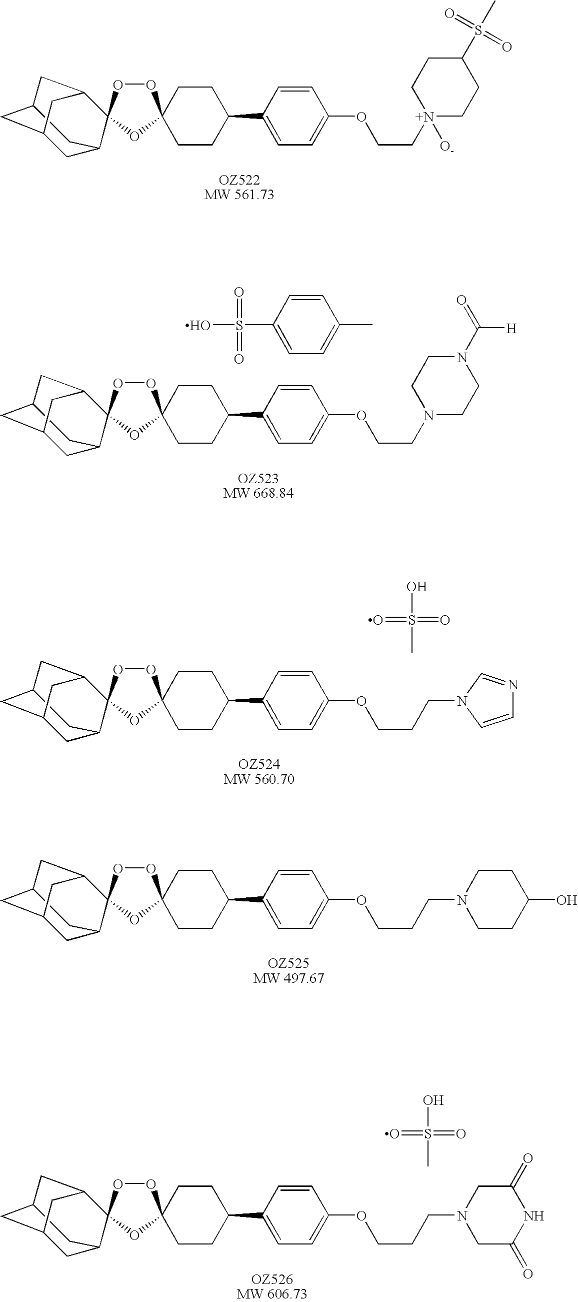

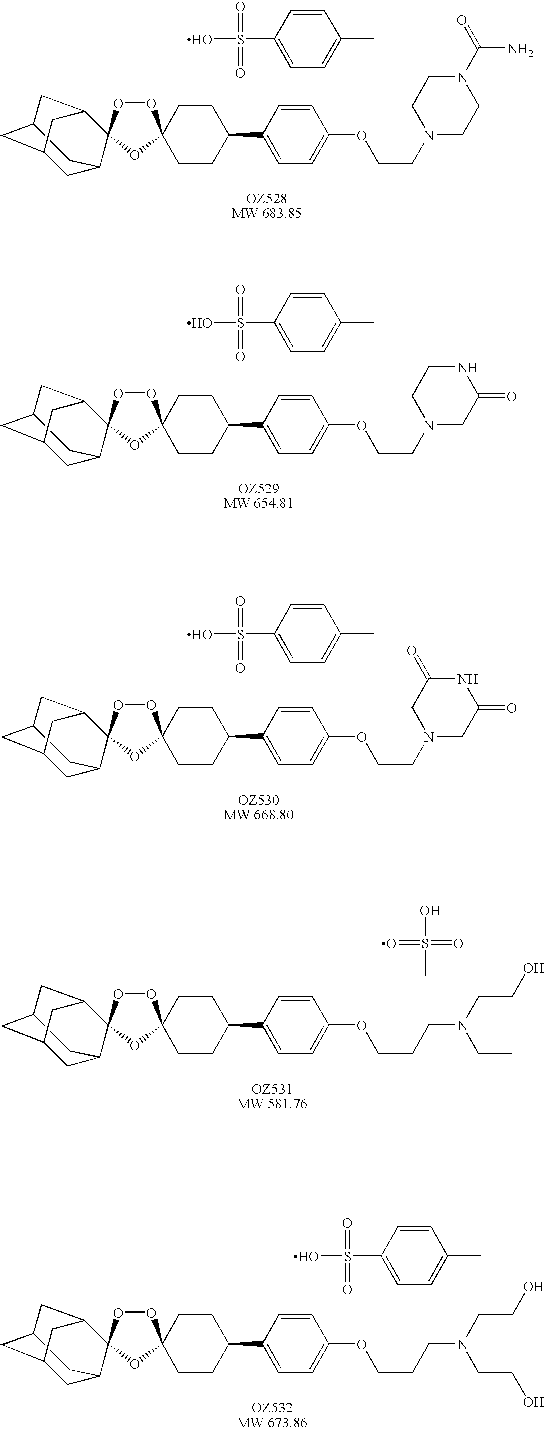

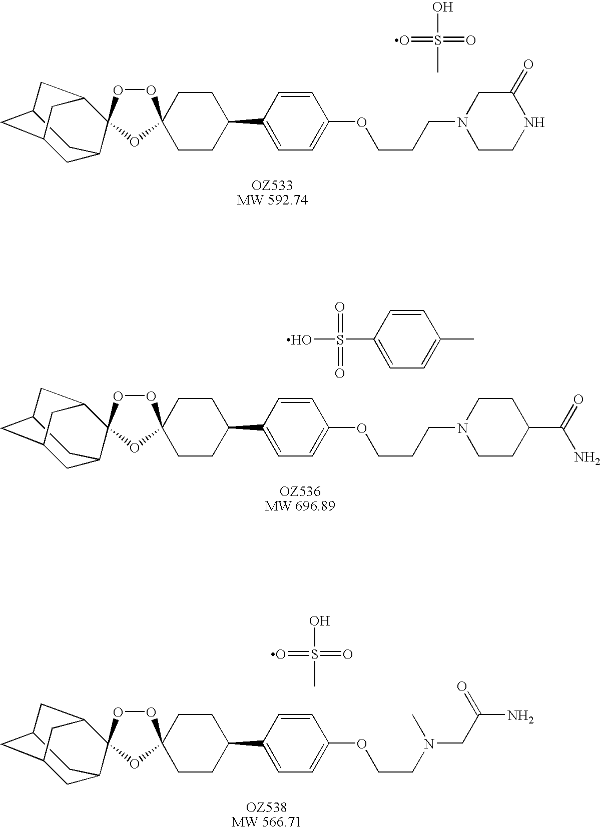

Preferred compounds of the present invention identified thus far include OZ413, OZ433, OZ434, OZ435, OZ436, OZ457, OZ465, OZ480, OZ481, OZ494, OZ501, and OZ511. It should be noted that OZ433, OZ434, and OZ435 are different salt forms of OZ401 (mesylate).

Notable features of these spiro and dispiro 1,2,4-trioxolanes in comparison to the artemisinin semisynthetic derivatives are their structural simplicity and ease of synthesis. For example, dispiro trioxolanes may be easily synthesized by the coozonolysis of the O-methyl oximes of cycloalkanones in the presence of the requisite cycloalkanone derivatives according to the method of Griesbaum et al. (1997a; 1997b) as illustrated below for the symmetrical dispiro cyclohexyl trioxolane:

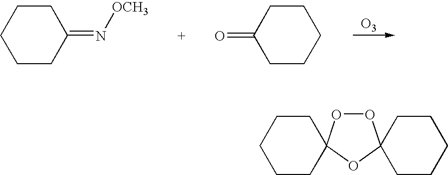

If yields are low in this coozonolysis reaction, yields can improve dramatically when the O-methyloxime and ketone are “reversed.” This novel procedure provides a uniquely convenient method to synthesize spiro and dispiro trioxolanes. Advantages of the oxime ether route over the alkene approach include convenient synthesis of starting materials (oxime ethers vs. tetrasubstituted alkenes), higher yield and selectivity of formation of desired trioxolanes by the judicious selection of paired reaction substrates. The trioxolanes may be purified by crystallization or by flash column chromatography. Their structures and purity may be confirmed by analytical HPLC, ¹H and ¹³C NMR, IR, melting point and elemental analysis.

Formation of a trioxolane from an oxime ether and a ketone is presumed to be a three-step process. The sequence begins by the electrophilic addition of ozone to the oxime double bond to form a primary ozonide. Second, the very unstable primary adduct fragments to a reactive carbonyl oxide driven in part by the concomitant expulsion of the relatively stable methyl nitrite. Third, the carbonyl oxide undergoes a [3+2]cycloaddition with a ketone to give the secondary ozonide or 1,2,4-trioxolane. It remains to be

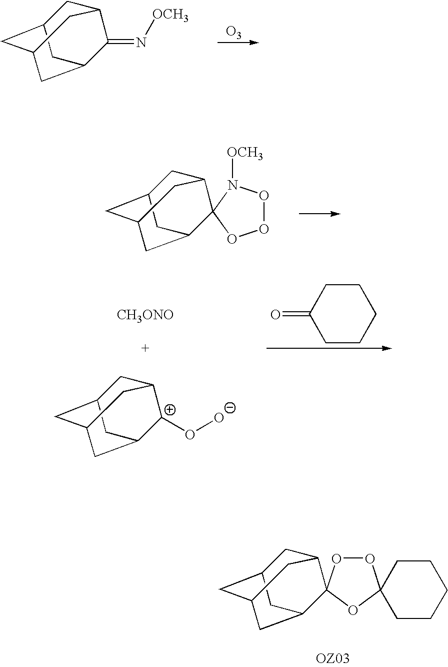

determined whether this is a stepwise or a concerted recombination process.

As illustrated above by the synthesis of OZ03, most of the new dispiro trioxolanes contain a spiroadamantane and can be synthesized by the coozonolysis of adamantanone O-methyl oxime in the presence of the requisite cycloalkanone derivative. The preferred reaction solvents for the coozonolysis reactions are hydrocarbon solvents such as pentane or cyclohexane; more polar solvents tend to decrease the yield of the reaction. When ketones are not readily soluble in pentane or cyclohexane, a mixed solvent (pentane/methylene chloride) or methylene chloride alone may be used. Several factors govern the ratio of oxime ether to ketone. In some reactions, in order to avoid diperoxide (1,2,4,5-tetraoxane) formation, to preclude diozonide formation from diketones, and to promote the reaction with readily pentane soluble ketones, excess ketone (2:1) is used. Most commonly in the discovery synthesis stage, and especially in cases where ketones are not readily soluble in pentane, expensive, or difficult to remove in the reaction workup, a 1:1 ratio of ketone to oxime ether may be used. In large scale trioxolane syntheses, a 1.5-fold excess of oxime ether can be used to achieve higher conversions of ketones into the desired product trioxolanes without causing purification problems.

The spiro and dispiro trioxolane compositions of the present invention may be generally used for the prophylaxis and treatment of malaria. The trioxolane compositions of the present invention are administered along with a pharmaceutically acceptable carrier. Any pharmaceutically acceptable carrier may be generally used for this purpose, provided that the carrier does not significantly interfere with the stability or bioavailability of the trioxolane compounds of this invention.

The trioxolanes of this invention can be administered in any effectively pharmaceutically acceptable form to warm blooded animals, including human and other animal subjects, e.g. in topical, lavage, oral, suppository, parenteral, or infusible dosage forms, as a topical, buccal, sublingual, or nasal spray or in any other manner effective to deliver the agents. The route of administration will preferably be designed to optimize delivery and/or localization of the agents to target cells.

In addition to the active compounds i.e. the trioxolanes, the pharmaceutical compositions of this invention may contain suitable excipients and auxiliaries which facilitate processing of the active compounds into preparations which can be used pharmaceutically. Oral dosage forms encompass tablets, capsules, and granules. Preparations which can be administered rectally include suppositories. Other dosage forms include suitable solutions for administration parenterally or orally, and compositions which can be administered buccally or sublingually.

The pharmaceutical preparations of the present invention are manufactured in a manner which is itself well known in the art. For example the pharmaceutical preparations may be made by means of conventional mixing, granulating, dragee-making, dissolving, lyophilizing processes. The processes to be used will depend ultimately on the physical properties of the active ingredient used.

Suitable excipients are, in particular, fillers such as sugars for example, lactose or sucrose mannitol or sorbitol, cellulose preparations and/or calcium phosphates, for example, tricalcium phosphate or calcium hydrogen phosphate, as well as binders such as starch, paste, using, for example, maize starch, wheat starch, rice starch, potato starch, gelatin, gum tragacanth, methyl cellulose, hydroxypropylmethylcellulose, sodium carboxymethylcellulose, and/or polyvinyl pyrrolidone. If desired, disintegrating agents may be added, such as the above-mentioned starches as well as carboxymethyl starch, cross-linked polyvinyl pyrrolidone, agar, or alginic acid or a salt thereof, such as sodium alginate. Auxiliaries are flow-regulating agents and lubricants, for example, such as silica, talc, stearic acid or salts thereof, such as magnesium stearate or calcium stearate and/or polyethylene glycol. Oral dosage forms may be provided with suitable coatings which, if desired, may be resistant to gastric juices.

For this purpose concentrated sugar solutions may be used, which may optionally contain gum arabic, talc, polyvinylpyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, dyestuffs and pigments may be added to the tablet coatings, for example, for identification or in order to characterize different combination of compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, liquid paraffin, or liquid polyethylene glycols. In addition stabilizers may be added. Possible pharmaceutical preparations which can be used rectally include, for example, suppositories, which consist of a combination of the active compounds with the suppository base. Suitable suppository bases are, for example, natural or synthetic triglycerides, paraffin hydrocarbons, polyethylene glycols, or higher alkanols. In addition, it is also possible to use gelatin rectal capsules which consist of a combination of the active compounds with a base. Possible base material include for example liquid triglycerides, polyethylene glycols, or paraffin hydrocarbons.

Suitable formulations for parenteral administration include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, including for example, sodium carboxymethyl cellulose, sorbitol and/or dextran. Such compositions may also comprise adjuvants such as preserving, wetting, emulsifying, and dispensing agents. They may also be sterilized, for example, by filtration through a bacteria-retaining filter, or by incorporating sterilizing agents into the compositions. They can also be manufactured in the form of sterile solid compositions which can be dissolved or suspended in sterile water, saline, or other injectable medium prior to administration.

In addition to administration with conventional carriers, active ingredients may be administered by a variety of specialized delivery drug techniques which are known to those of skill in the art, such as portable infusion pumps.

The trioxolane compositions of the present invention are administered along with a pharmaceutically acceptable carrier in an amount sufficient to prevent malarial infection and/or treat an active infection. The trioxolane compounds of this invention have extremely low toxicity and a low degree of side effects even at high doses. The dosing range of the trioxolane compositions will vary depending on a number of factors, such as whether it is used for prophylaxis or treatment of an active infection, route of administration, dosing schedule, etc. In general, the therapeutic dose of trioxolane may range between about 0.1-1000 mg/kg/day, with between about 1-100 mg/kg/day being preferred. The foregoing doses may be administered as a single dose or may be divided into multiple doses for administration. For single dosing, a preferred dosing range is from about 0.5-5.0 mg/kg.

The trioxolane compositions may be administered once to several times daily. For malaria prevention, a typical dosing schedule could be, for example (other than for single dose cure), 2.0-1000 mg/kg weekly beginning 1-2 weeks prior to malaria exposure taken up until 1-2 weeks post-exposure.

The spiro and dispiro trioxolanes of this invention may be administered as any pharmaceutically effective salt form. Such salts are well known in the art and include, but are not limited to acetate, adipate, alginate, citrate, aspartate, benzoate, benzene sulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluene sulfonate and undecanoate salts. Preferred salts are those that increase the bioavailability of the trioxolane compounds. This will depend upon a number of factors, including the chemical structure of the trioxolane, the carrier to which it is incorporated, the route of administration, etc.

As a general rule, it is preferred to administer the compounds of the present invention, as well as other antimalarials, as part of a combination therapy in order to prevent resistance. The benefits of such combination therapy in treating malaria are well known in the art. Combination therapy with antimalarial drugs is the simultaneous use of two or more blood schizontocidal drugs with independent modes of action and different biochemical targets in the parasite. The concept of combination therapy is based on the synergistic or additive potential of two or more drugs, to improve therapeutic efficacy and also delay the development of resistance to the individual components of the combination. Examples of current antimalarial drug combinations include artemisinin combinations with chloroquine and mefloquine and quinine based combinations with tetracycline and clindamycin. The trioxolanes of the present invention are expected to be administered in combinations with various other antimalarials including, but not limited to, artemether, chloroquine, mefloquine, and piperaquine.

The spiro and dispiro trioxolanes of this invention have been found to be effective in the treatment of schistosomiasis. Schistosomiasis ranks second behind malaria in terms of socioeconomic and public health importance in tropical and subtropical areas. The disease is endemic in 74 developing countries, infecting more than 200 million people in rural agricultural and peri-urban areas. An estimated 500-600 million people worldwide are at risk from the disease.

The major forms of human schistosomiasis are caused by five species of water-borne flatworm, or blood flukes, called schistosomes. One of these species is Schistosoma mansoni, which has been reported in 53 countries in Africa, the Eastern Mediterranean, the Caribbean, and South America. The parasites enter the body through contact with infested surface water, primarily among people engaged in agriculture and fishing. The parasites normally infect the host during the cercaria, or larval stage. Once inside the host, the cercaria develop into adults or schistosomes.

Current treatments for schistosomiasis have focused primarily on prophylaxis, i.e. prevention of host infection by cercaria. Currently, praziquantel is the most widely used drug for treatment of schistosomiasis. While artemether has demonstrated activity in the prophylaxis of schistosomiasis, it has not shown any activity against adult S. mansoni.

It has now been unexpectedly discovered that the spiro and dispiro trioxolanes of this invention are active against both cercaria and adult S. mansoni, S. japonicum when administered in the dosages and manner outlined above with respect to treatment of malarial parasites. It is also believed the trioxolanes of this invention will be active against S. haematobium. Preferred compounds of this invention identified for use in the treatment of schistosomiasis include OZ472 and OZ509. Preferred dosing levels of the spiro and dispiro trioxolanes are about 100-200 mg/kg/day orally.

The spiro and dispiro trioxolanes of this invention have further been found to be effective in the treatment of fascioliasis. Fascioliasis is a zoonotic disease, which is of considerable public health and great veterinary significance. The causative agent of fascioliasis is a liver fluke, i.e., Fasciola hepatica and F. gigantica. Human fascioliasis occurs worldwide, with the highest number of infected people reported in South America, Cuba, Western Europe, Egypt and the Islamic Republic of Iran. An estimated 91 million people are at risk and as many as 17 million people might be infected with either F. hepatica or F. gigantica (WHO, 1995; Esteban et al., 2003). Treatment of fascioliasis has thus far relied upon a single drug—triclabendazole. However, this drug is currently registered in only 4 countries and, hence, novel treatment options must be pursued. There is considerable concern about the development of resistance to triclabendazole, which is already common in sheep and cattle.

It has now been unexpectedly discovered that the spiro and dispiro trioxolanes of this and the inventor's prior inventions are active against both Fasciola hepatica and F. gigantica when administered in the dosages and manner outlined above with respect to treatment of malarial parasites. These trioxolane compounds include those of the general chemical structure described in the inventors' previous applications:

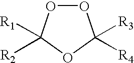

wherein R₁, R₂, R₃, and R₄ are the same or different, and are selected from the group consisting of substituted or unsubstituted linear or branched alkyl, aryl, and alkaryl groups and substituted or unsubstituted alicyclic groups that may be interrupted by one or more oxygen, sulfur or nitrogen atoms, and substituted or unsubstituted aromatic or heterocyclic groups, whereby none of R₁, R₂, R₃, or R₄ may be hydrogen; and further providing that R₁ and R₂ taken together and/or R₃ and R₄ taken together may form a substituted or unsubstituted alicyclic group which is optionally interrupted by one or more oxygen, sulfur or nitrogen atoms.

Preferred compounds identified for use in the treatment of fascioliasis include cis-Adamantane-2-spiro-3′-8′-carboxymethyl-1′,2′,4′-trioxaspiro[4.5]decane (OZ78, the subject of PCT/US02/19767), cis-Adamantane-2-spiro-3′-8′-(4′-hydroxyphenyl)-1′,2′,4′-trioxaspiro[4.5]decane (OZ288, the subject of U.S. Pat. No. 6,825,230), cis-Adamantane-2-spiro-3′-8′-(2′-carboxyethyl)-1′,2′,4′-trioxaspiro[4.5]decane (OZ352), the subject of U.S. Pat. No. 6,906,205), and OZ418. Preferred dosing levels of the spiro and dispiro trioxolanes for treatment of fascioliasis are about 100-200 mg/kg/day orally.

Other drugs besides trioxolanes which are compatible with the carrier ingredients may also be incorporated into the carrier. Such drugs may be readily ascertained by those of ordinary skill in the art and may include, for instance, antibiotics, other antimalarials, antiinflammatory agents, etc.

It is understood that the present invention contemplates the use of not only the above-stated trioxolane compounds themselves, but their prodrugs which metabolize to the compound and the analogues and biologically active salt forms thereof, as well as optical isomers which provide the same pharmaceutical results.

The following examples are offered to illustrate but not limit the invention. Thus, they are presented with the understanding that various formulation modifications as well as method of delivery modifications may be made and still be within the spirit of the invention.

### Example 1

### Antimalarial Activity of New OZ Compounds

Activity of 1,2,4-trioxolanes against P. falciparum in vitro. Each trioxolane was screened against the chloroquine-resistant K1 and chloroquine-sensitive NF54 strains of Plasmodium falciparum in vitro.

Activity of 1,2,4-trioxolanes against P. berghei in vivo. In the single dose in vivo screen, Moro or NMRI mice infected with the ANKA strain of P. berghei (groups of five mice) were treated one day post-infection with trioxolanes dissolved or suspended in standard suspending vehicle (SSV). The SSV consists of 0.5% w/v CMC, 0.5% v/v benzyl alcohol, 0.4% v/v Tween 80, and 0.9% w/v sodium chloride in water. Trioxolanes were administered as single po 3 or 30 mg/kg doses. Trioxolanes were also administered as single po 100 mg/kg doses in a tween/ethanol (T/A) vehicle. The T/A consists of 3% ethanol and 7% Tween 80. Antimalarial activity was measured by percent reduction in parasitemia on day three post-infection and survival times compared to an untreated control group. Survival to day 30 post-infection is considered to be a cure.

Comparative data for the antimalarial drug controls artesunate (AS), artemether (AM), chloroquine (CQ), and mefloquine (MQ) are also included.

Below is the activity data for the OZ compounds of the invention.

TABLE 1

| | | Activity (%) | Survival (days) | Cures |
|---|---|---|---|---|
| | IC₅₀ (ng/ml) | 3 and 30 mg/kg | 3 and 30 mg/kg | 30 mg/kg |
| Compd | K1/NF54 | SSV po | SSV po | SSV po |
| Control | — | 0 | 6-7 | — |
| OZ406 | 0.72/0.62 | 96/99.7 | 6.0/14.4 | 0/5 |
| OZ407 | 2.6/2.4 | 99.23¹ | 10.3¹ | ND |
| OZ408 | 1.1/1.0 | 40/ND | 6.4/ND | ND |
| OZ409 | 0.43/0.40 | 98/>99.9 | 6.2/9.0 | 0/5 |
| OZ410 | 1.8/2.5 | 99.47¹ | 7.7¹ | ND |
| OZ411 | 23/37 | 22¹ | 6.7¹ | ND |
| OZ412 | >100/>100 | 4¹ | 6.0¹ | ND |
| OZ413 | 1.0/0.77 | 94/99.6 | 6.0/27.4 | 2/5 |
| OZ414 | 0.80/0.59 | 86/>99.9 | 6.6/14.2 | 0/5 |
| OZ415 | 84/150 | 0/ND | 5.8/ND | ND |
| OZ416 | 100/110 | 0/ND | 5.8/ND | ND |
| OZ417 | 150/180 | 0/ND | 6.2/ND | ND |
| OZ418 | 93/100 | 0/ND | 5.8/ND | ND |
| OZ419 | 4.6/5.5 | 19/ND | 5.8/ND | ND |
| OZ420 | 1.6/1.6 | 93/99.44 | 5.6/9.2 | 0/5 |
| OZ421 | 2.8/2.9 | 38/ND | 6.4/ND | ND |
| OZ422 | 2.8/2.9 | 34/ND | 6.0/ND | ND |
| OZ423 | 2.3/2.3 | 69/99.69 | 6.6/7.4 | 0/5 |
| OZ424 | 0.68/1.8 | 96/99.64 | 6.0/11.0 | 0/5 |
| OZ425 | 0.76/2.0 | 97/99.23 | 5.8/14.0 | 0/5 |
| OZ426 | 0.59/1.4 | 98/99.79 | 6.0/8.0 | 0/5 |
| OZ427 | 0.39/1.2 | 93/99.07 | 5.6/9.2 | 0/5 |
| OZ428 | 0.36/1.2 | 95/99.89 | 5.8/7.4 | 0/5 |
| OZ430 | 0.88/1.8 | 72/98.47 | 5.8/7.0 | 0/5 |
| OZ431 | 0.58/1.5 | 96/99.62 | 5.8/11.6 | 0/5 |
| OZ432 | 0.69/1.8 | 86/99.43 | 5.8/9.4 | 0/5 |
| OZ433 | 1.2/1.8 | 99/98 | 6.6/24.2 | 2/5 |
| OZ434 | 0.77/1.4 | 98/98 | 6.2/22.4 | 2/5 |
| OZ435 | 1.3/1.5 | 98/98 | 6.2/27.6 | 4/5 |
| OZ436 | 1.2/1.9 | 98/98 | 6.0/24.2 | 1/5 |
| OZ437 | 1.0/1.5 | 99/99 | 6.0/15.6 | 0/5 |
| OZ438 | 1.1/1.8 | 99/99 | 6.0/14.0 | 0/5 |
| OZ440 | 90/140 | 0/ND | 6.0/ND | ND |
| OZ441 | 1.2/1.7 | 15/ND | 6.4/ND | ND |
| OZ442 | 0.58/0.79 | 76/97 | 6.6/7.0 | 0/5 |
| OZ443 | 0.98/1.1 | 98/98 | 6.6/16.4 | 0/5 |
| OZ444 | 24/29 | 44/ND | 6.4/ND | ND |
| OZ445 | 0.86/1.0 | 99/98 | 6.6/15.6 | 0/5 |
| OZ446 | 18/20 | 98/98 | 6.8/6.8 | 0/5 |
| OZ447 | 1.7/1.9 | 17/ND | 6.8/ND | ND |
| OZ448 | 2.6/3.1 | 57/98 | 7.0/8.8 | 0/5 |
| OZ449 | 1.4/1.7 | 24/ND | 6.0/ND | ND |
| OZ450 | 3.2/4.0 | 87/96 | 6.4/13.4 | 0/5 |
| OZ451 | 0.30/0.40 | 30/ND | 7.4/ND | ND |
| OZ452 | 8.2/12 | 0/ND | 6.2/ND | ND |
| OZ454 | 2.7/3.0 | 0/ND | 6.2/ND | ND |
| OZ455 | 0.76/1.2 | 98/99.6 | 6.2/13.0 | 0/5 |
| OZ456 | 0.54/0.90 | 98¹ | 9.4¹ | ND |
| OZ457 | 0.97/1.3 | 98/>99.9 | 6.6/22.2 | 2/5 |
| OZ458 | 2.4/3.0 | 57/99.8 | 8.4/16.0 | 0/5 |
| OZ459 | 4.0/5.5 | 17/ND | 7.0/ND | ND |
| OZ463 | 3.8/4.4 | 45/99.6 | 6.0/14.8 | 0/5 |
| OZ465 | 3.0/2.8 | 10/99.5 | 6.0/10.4 | 0/5 |
| OZ467 | 1.7/1.9 | 79¹ | 5.4¹ | ND |
| OZ468 | 36/50 | 97¹ | 5.4¹ | ND |
| OZ469 | 1.4/1.3 | 6/98 | 6.0/5.2 | 0/5 |
| OZ470 | 1.5/1.3 | 0/0 | 6.0/6.0 | 0/5 |
| OZ471 | 1.0/1.1 | 40¹ | 6.2¹ | ND |
| OZ472 | 1.5/1.5 | 99.0¹ | 9.8¹ | ND |
| OZ473 | 7.9/12 | 99¹ | 7.6¹ | ND |
| OZ474 | 2.5/1.9 | 99¹ | 10.0¹ | ND |
| OZ475 | 1.8/1.8 | 99.3¹ | 8.2¹ | ND |
| OZ476 | 1.6/3.3 | 19¹ | 6.0¹ | ND |
| OZ477 | 0.80/1.2 | 0/96 | 6.0/5.8 | 0/5 |
| OZ478 | 10/8.6 | ND/4 | ND/6.0 | 0/5 |
| OZ479 | 6.2/3.8 | 79/99.4 | 6.0/15.4 | 0/5 |
| OZ480 | 5.2/4.8 | 5/99.7 | 6.0/24.8 | 3/5 |
| OZ481 | 3.6/3.6 | 87/99.7 | 5.8/22.0 | 1/5 |
| OZ483 | 2.8/2.8 | 99.7¹ | 21.6¹ | ND |
| OZ484 | 6.6/5.8 | 5/99.7 | 6.0/10.6 | 0/5 |
| OZ486 | 15/14 | ND/90 | ND/6.6 | 0/5 |
| OZ487 | 1.6/3.4 | 99.1/99.6 | 6.0/16.0 | 0/5 |
| OZ488 | 3.2/5.6 | ND/99.6 | ND/10.0 | 0/5 |
| OZ489 | 3.3/5.8 | 11/99.5 | 6.0/14.6 | 0/5 |
| OZ490 | 3.7/6.9 | ND/99.1 | ND/6.8 | 0/5 |
| OZ491 | 1.2/2.5 | ND/99.5 | ND/12.6 | 0/5 |
| OZ492 | 0.62/1.7 | 99.4¹ | 14.8¹ | ND |
| OZ494 | 0.90/1.8 | 99.2/99.6 | 6.0/22.8 | 1/5 |
| OZ496 | 0.88/1.1 | ND/99.7 | ND/14.6 | 0/5 |
| OZ497 | 0.97/0.97 | ND/99.3 | ND/14.8 | 0/5 |
| OZ500 | 1.1/2.1 | 97¹ | 6.2¹ | ND |
| OZ501 | 1.1/2.1 | ND/99.6 | ND/21.2 | 1/5 |
| OZ502 | 2.9/4.8 | ND/99.7 | ND/13.6 | 0/5 |
| OZ503 | 1.1/1.7 | ND/99.6 | ND/14.8 | 0/5 |
| OZ504 | 3.1/5.4 | ND/99.7 | ND/14.4 | 0/5 |
| OZ506 | 3.8/3.2 | ND/99.9 | ND/13.6 | 0/5 |
| OZ507 | 1.2/1.1 | ND/99.8 | ND/11.6 | 0/5 |
| OZ509 | 0.72/0.53 | ND/99.7 | ND/12.4 | 0/5 |
| OZ510 | 2.4/2.7 | ND/99.9 | ND/13.0 | 0/5 |
| OZ511 | 2.0/1.8 | ND/99.8 | ND/24.2 | 3/5 |
| OZ512 | 1.2/0.83 | ND/99.7 | ND/12.2 | 0/5 |
| OZ514 | 2.0/2.0 | ND/99.5 | ND/8.4 | 0/5 |
| OZ515 | 2.6/2.0 | ND/99.8 | ND/12.0 | 0/5 |
| OZ516 | 9.5/13 | 99.1¹ | 6.6¹ | ND |
| OZ517 | 1.2/0.85 | ND/99.6 | ND/13.6 | 0/5 |
| OZ518 | 1.3/0.99 | ND/99.7 | ND/15.0 | 0/5 |
| OZ519 | 0.83/0.59 | ND/99.7 | ND/13.8 | 0/5 |
| OZ520 | 1.6/1.4 | ND/99.6 | ND/15.0 | 0/5 |
| OZ521 | 1.9/1.5 | ND/99.7 | ND/16.6 | 0/5 |
| OZ522 | 2.3/2.3 | ND/99.8 | ND/13.4 | 0/5 |
| OZ523 | 2.4/2.0 | ND/99.7 | ND/14.6 | 0/5 |
| OZ524 | 2.0/1.6 | ND/99.9 | ND/14.8 | 0/5 |
| OZ525 | 1.2/0.93 | ND/99.6 | ND/15.8 | 0/5 |
| OZ526 | 5.7/5.4 | ND/99.3 | ND/6.0 | 0/5 |
| OZ528 | 1.9/2.0 | ND/99.6 | ND/14.0 | 0/5 |
| OZ529 | 3.2/3.4 | ND/99.7 | ND/10.2 | 0/5 |
| OZ530 | 7.2/7.3 | ND/99.0 | ND/7.0 | 0/5 |
| OZ531 | 1.4/1.1 | ND/99.7 | ND/16.0 | 0/5 |
| OZ532 | 1.4/1.2 | ND/99.8 | ND/16.0 | 0/5 |
| OZ533 | 2.2/2.5 | ND/99.4 | ND/15.6 | 0/5 |
| OZ536 | 1.3/1.1 | ND/99.6 | ND/15.8 | 0/5 |
| OZ538 | 1.6/1.6 | ND/99.6 | ND/14.0 | 0/5 |
| AS | 1.3/1.6 | 33/92 | 6.6/9.0 | 0/5 |
| AM | 0.74/1.2 | 56/99.7 | 8.0/9.0 | 0/5 |
| CQ | 62/5.1 | 85/99.9 | 7.9/9.6 | 0/5 |
| MQ | 3.0/5.8 | 18/99.6 | 7.0/21.8 | 0/5 |

¹data at a single po 100 mg/kg dose (T/A)

The new activity data demonstrates that the trioxolane compounds of the invention demonstrate the best potency and good oral activity against malarial parasites.

### Example 3

### Preferred Procedures for Preparation of Compounds

cis-Adamantane-2-spiro-3′-8′-[4′-(aminomethyl)phenyl]-1′,2′,4′-trioxaspiro[4.5]decane mesylate (OZ406). Step 1. To a stirred solution of 1 M TiCl₄ in CH₂Cl₂ (125 ml, 125 mmol) at 0° C. was added a solution of 4-phenylcyclohexanone (8.72 g, 50 mmol) in CH₂Cl₂ (40 ml) followed by a solution of chloromethyl methyl ether (6.04 g, 75 mmol) in CH₂Cl₂ (20 ml). After 1 h, an additional amount of chloromethyl methyl ether (6.04 g, 75 mmol) in CH₂Cl₂ (20 ml) was added dropwise. After being stirred for an additional 1.5 h, the reaction mixture was poured into 300 ml of 12% aq. HCl. The organic phase was separated, washed with water, aqueous NaHCO₃, water, and brine, and dried over MgSO₄. After removal of the solvent, the residue was purified by chromatography (silica gel, chloroform) followed by crystallization from hexane-chloroform (4:1) at 0° C. to give the chloromethyl ketone (2.5 g, 23%). ¹H NMR (500 MHz, CDCl₃) δ 1.89-1.97 (m, 2H), 2.19-2.22 (m, 2H), 2.49-2.52 (m, 4H), 3.00-3.06 (m, 1H), 4.57 (s, 2H), 7.24 (d, J=7.8 Hz, 2H), 7.35 (d, J=7.8 Hz, 2H). Step 2. A solution of O-methyl 2-adamantanone oxime (0.969 g, 5.41 mmol) and 4-[(4-chloromethyl)phenyl]cyclohexanone (0.80 g, 3.6 mmol) in cyclohexane (90 ml) and CH₂Cl₂ (10 ml) was treated with ozone according to the general procedure. After removal of the solvents, EtOH (30 ml) was added. The resulting precipitate was filtered, washed with 50% aq. EtOH (20 ml), and dried to give the first crop of chloromethyl trioxolane (0.250 g) as a colorless solid. Crystallization of the remaining mother liquor from EtOH (30 ml) at 0° C. furnished the second crop of chloromethyl trioxolane (0.650 g) as a colorless solid, with a combined yield of 65%. ¹H NMR (500 MHz, CDCl₃) δ 1.69-2.06 (m, 22H), 2.52-2.57 (m, 1H), 4.56 (s, 2H), 7.20 (d, J=7.8 Hz, 2H), 7.31 (d, J=7.8 Hz, 2H). Step 3. To a solution of the above chloromethyl trioxolane (0.70 g, 1.80 mmol) in dry DMF (25 ml) at rt under Ar was added sodium azide (0.15 g, 2.35 mmol) portion-wise. After the addition, the reaction mixture was heated at 60° C. overnight. After the mixture was cooled to rt and poured onto chopped ice. The resulting solid was filtered, washed with water (50 ml), and dried to afford the azido trioxolane (0.65 g, 92%) as a colorless solid. mp 110-112° C.; ¹H NMR (500 MHz, CDCl₃) δ 1.70-2.06 (m, 22H), 2.53-2.58 (m, 1H), 4.30 (s, 2H), 7.21-7.26 (m, 4H). Step 4. To a solution of the above azido trioxolane (0.625 g, 1.58 mmol) in THF (30 ml) were added triphenylphosphine (0.50 g, 1.9 mmol) and water (2 ml). The reaction mixture was stirred at rt overnight. After removal of the solvents, the residue was dissolved in CH₂Cl₂/EtOAc (1:1, 20 ml). Then a solution of methanesulfonic acid (0.12 g, 1.27 mmol) in ether (5 ml) was added to the above solution at 0° C. The solid obtained was filtered and washed with ethyl acetate (20 ml), ether (20 ml), and dried to afford trioxolane OZ406 (0.64 g, 87%) as a colorless solid. mp 137-141° C.; ¹H NMR (500 MHz, DMSO-d₆) δ 1.45-2.02 (m, 22H), 2.35 (s, 3H), 2.60-2.69 (m, 1H), 3.98 (s, 2H), 7.27 (d, J=7.8 Hz, 2H), 7.39 (d, J=8.3 Hz, 2H), 8.14 (brs, 3H); ¹³C NMR (125.7 MHz, DMSO-d₆) δ 26.01, 26.42, 31.21, 34.20, 34.45, 35.98, 36.28, 39.91, 41.44, 42.25, 108.25, 110.77, 127.02, 129.24, 131.96, 146.53. Anal. Calcd for C₂₄H₃₅NO₆S: C, 61.91; H, 7.58; N, 3.01. Found: C, 61.84; H, 7.80; N, 2.92.

Adamantane-2-spiro-3′-5′-cyclohexyl-5′-methyl-1′,2′,4′-trioxolane (OZ407). A solution of O-methyl 2-adamantanone oxime (2.7 g, 15 mmol) and cyclohexyl methyl ketone (3.8 g, 30 mmol) in cyclohexane (100 ml) and CH₂Cl₂ (20 ml) was treated with ozone according to the general procedure. After removal of solvents, the crude product was purified by chromatography (silica gel, 10% ether in hexanes) to afford trioxolane OZ407 (1.15 g, 26%) as a colorless oil. ¹H NMR (500 MHz, CDCl₃) δ 1.02-1.31 (m, 5H), 1.36 (s, 3H), 1.61-2.09 (m, 20H); ¹³C NMR (125.7 MHz, CDCl₃) δ 20.14, 26.16, 26.32, 26.33, 26.49, 26.89, 27.66, 27.84, 34.67, 34.75, 34.79, 34.98, 36.15, 36.54, 36.81, 45.21, 111.24, 111.64. Anal. Calcd for C₁₈H₂₈O₃: C, 73.93; H, 9.65. Found: C, 74.00; H, 9.42.

cis-Adamantane-2-spiro-3′-8′-(3′-hydroxypropyl)-1′,2′,4′-trioxaspiro[4.5]decane (OZ408). To a solution of the methyl ester of OZ352 (0.90 g, 2.57 mmol) in ether (5 ml) and THF (1 ml) was added dropwise 2 M lithium borohydride in THF (1.29 ml, 2.58 mmol) followed by 1 M lithium triethylborohydride in THF (0.26 ml, 0.26 mmol). The resulting mixture was stirred at rt for 3 h and then diluted with ether (30 ml). The mixture was washed with 2 M aqueous NaOH (2×5 ml), water (2×5 ml) and brine (5 ml), dried over MgSO₄, filtered, and concentrated to afford trioxolane OZ408 (0.83 g, 100%) as a colorless solid. mp 68-70° C.; ¹H NMR (500 MHz, CDCl₃) δ 1.11-1.32 (m, 4H), 1.49-2.07 (m, 23H), 3.62 (brs, 2H); ¹³C NMR (125.7 MHz, CDCl₃) δ 26.48, 26.88, 30.09, 30.34, 32.21, 34.20, 34.78, 34.80, 35.94, 36.39, 36.81, 63.22, 109.01, 111.18. Anal. Calcd for C₁₉H₃₀O₄: C, 70.77; H, 9.38. Found: C, 70.79; H, 9.18.

cis-Adamantane-2-spiro-3′-8′-(3′-aminopropyl)-1′,2′,4′-trioxaspiro[4.5]decane mesylate (OZ409). Step 1. Diisopropyl azodicarboxylate (0.68 ml, 3.36 mmol) was added dropwise to a mixture of OZ408 (0.83 g, 2.58 mmol), phthalimide (0.38 g, 2.58 mmol), and triphenylphosphine (0.88 g, 3.36 mmol) in THF (20 ml) at 0° C. under Ar. The resulting mixture was stirred at rt for 24 h and then quenched with 5% aqueous NaHCO₃ (20 ml). The solid was collected by filtration and washed with water, THF, and ether to afford the phthalimido trioxolane (0.95 g, 82%) as a colorless solid. mp 138-139° C. ¹H NMR (500 MHz, CDCl₃) δ1.10-1.32 (m, 6H), 1.60-2.01 (m, 21H), 3.66 (t, J=7.3 Hz, 2H), 7.68-7.74 (m, 2H), 7.81-7.87 (m, 2H). Step 2. A mixture of the above phthalimido trioxolane (0.90 g, 2.00 mmol) and hydrazine monohydrate (1.5 ml) in chloroform (30 ml) and methanol (4 ml) was heated at 50° C. for 24 h. The reaction mixture was cooled to rt, filtered to remove the solid by-product, and concentrated. The residue was dissolved in CH₂Cl₂ (30 ml), washed with brine, dried over MgSO₄, filtered, and concentrated. The residue was dissolved in CH₂Cl₂ (5 ml) and then the solution of methanesulfonic acid (0.20 g) in ether (20 ml) was added. The precipitate was collected by filtration to afford trioxolane OZ409 (0.63 g, 76%) as a colorless solid. mp 152-154° C.; ¹H NMR (500 MHz, DMSO-d₆) δ 0.98-1.11 (m, 2H), 1.15-1.23 (m, 2H), 1.24-1.35 (m, 1H), 1.45-1.57 (m, 2H), 1.59-1.94 (m, 20H), 2.29 (s, 3H), 2.69-2.78 (m, 2H), 7.60 (brs, 3H); ¹³C NMR (125.7 MHz, DMSO-d₆) δ 24.75, 25.96, 26.37, 29.72, 32.32, 33.66, 34.41, 34.43, 34.77, 35.93, 36.24, 39.79, 39.94, 108.77, 110.61. Anal. Calcd for C₂₀H₃₅NO₆S: C, 57.53; H, 8.45; N, 3.35. Found: C, 57.70; H, 8.58; N, 3.26.

Adamantane-2-spiro-3′-5′,5′-dimethyl-1′,2′,4′-trioxolane (OZ410). A solution of O-methyl 2-adamantanone oxime (2.70 g, 15 mmol) and acetone (1.80 g, 31 mmol) in cyclohexane (150 ml) was treated with ozone according to the general procedure. After removal of the solvent, the crude product was purified by chromatography (silica gel, 2% EtOAc in hexanes) to afford trioxolane OZ410 (2.34 g, 69%) as a colorless oil. ¹H NMR (500 MHz, CDCl₃) δ 1.47 (s, 6H), 1.61-2.08 (m, 14H); ¹³C NMR (125.7 MHz, CDCl₃) δ 24.99, 26.47, 26.88, 34.77, 34.81, 36.19, 36.83, 108.30, 111.74. Anal. Calcd for C₁₃H₂₀O₃: C, 69.61; H, 8.99. Found: C, 69.78; H, 9.13.

3-Methyl-3-phenyl-1,2,4-trioxaspiro[4.5]decane (OZ411). A solution of O-methyl cyclohexanone oxime (1.27 g, 10 mmol) and acetophenone (2.4 g, 20 mmol) in cyclohexane (150 ml) and CH₂Cl₂ (60 ml) was treated with ozone according to the general procedure. After removal of solvents, the crude product was purified by chromatography (silica gel, 2% EtOAc in hexanes) to afford trioxolane OZ411 (0.90 g, 38%) as a colorless oil. ¹H NMR (500 MHz, CDCl₃) δ 1.38-1.78 (m, 8H), 1.72 (s, 3H), 1.79-1.95 (m, 2H), 7.30-7.39 (m, 3H), 7.49-7.54 (m, 2H); ¹³C NMR (125.7 MHz, CDCl₃) δ 23.70, 23.91, 24.89, 25.76, 33.44, 35.14, 108.57, 110.46, 125.19, 128.08, 128.15, 142.36. Anal. Calcd for C₁₄H₁₈O₃: C, 71.77; H, 7.74. Found: C, 71.62; H, 7.81.

3-Phenyl-1,2,4-trioxaspiro[4.5]decane (OZ412). A solution of O-methyl benzaldehyde oxime (1.70 g, 12.6 mmol) and cyclohexanone (2.50 g, 20 mmol) in cyclohexane (150 ml) and CH₂Cl₂ (60 ml) was treated with ozone according to the general procedure. After removal of the solvents, the crude product was purified by chromatography (silica gel, hexane) to afford trioxolane OZ412 (0.62 g, 22%) as a colorless oil. ¹H NMR (500 MHz, CDCl₃) δ 1.41-1.52 (m, 2H), 1.61-1.80 (m, 4H), 1.81-1.95 (m, 4H), 6.07 (s, 1H), 7.35-7.45 (m, 3H), 7.49-7.55 (m, 2H); ¹³C NMR (125.7 MHz, CDCl₃) δ 23.60, 23.96, 24.88, 33.38, 35.53, 103.45, 110.56, 127.78, 128.55, 130.35, 132.60. Anal. Calcd for C₁₃H₁₆O₃: C, 70.89; H, 7.32. Found: C, 70.69; H, 7.35.

cis-Adamantane-2-spiro-3′-8′-[4′-[[[(2′-amino-2′-methylpropyl)amino]carbonyl]oxy]phenyl]-1′,2′,4′-trioxaspiro[4.5]decane mesylate (OZ413). Step 1. To a solution of OZ288 (0.60 g, 1.69 mmol) and triethylamine (0.26 g, 2.52 mmol) in CH₂Cl₂ (30 ml) at 0° C. was added 4-nitrophenyl chloroformate (0.41 g, 2.02 mmol). After the reaction mixture was stirred overnight, it was quenched by the addition of water (25 ml). After separation of the organic layer, the aqueous layer was extracted with CH₂Cl₂ (2×10 ml). The combined extracts were washed with water and brine, dried over MgSO₄, and concentrated to afford the trioxolane carbonate (0.865 g, 99.9%) as a colorless solid, which was used for the next step without further purification. ¹H NMR (500 MHz, CDCl₃) δ 1.69-2.07 (m, 22H), 2.54-2.63 (m, 1H), 7.18 (d, J=8.3 Hz, 2H), 7.26 (d, J=8.3 Hz, 2H), 7.47 (d, J=8.8 Hz, 2H), 8.29 (d, J=9.3 Hz, 2H). Step 2. To a solution of the above p-nitrophenyl carbonate (0.865 g, 1.7 mmol) in CH₂Cl₂ (20 ml) was added rapidly a solution of 1,2-diamino-2-methylpropane (0.73 g, 8.3 mmol) in CH₂Cl₂ (5 ml). The reaction mixture was stirred at rt overnight and then quenched with water (15 ml). After separation of the organic layer, the aqueous layer was extracted with CH₂Cl₂ (2×10 ml). The combined extracts were washed with water (3×25 ml), dried over MgSO₄, and concentrated to afford the free base as a viscous solid. To the solution of the above free base in CH₂Cl₂ (5 ml) at 0° C. was added a solution of methanesulfonic acid (0.14 g, 1.44 mmol) in ether (5 ml). The solid obtained was filtered, washed with ether (20 ml), and dried to afford trioxolane OZ413 (0.694 g, 72%) as a colorless solid. mp 148-152° C.; ¹H NMR (500 MHz, DMSO-d₆) δ 1.23 (s, 6H), 1.49-2.01 (m, 22H), 2.34 (s, 3H), 2.59-2.68 (m, 1H), 3.22 (d, J=6.3 Hz, 2H), 7.07 (d, J=8.3 Hz, 2H), 7.22 (d, J=8.3 Hz, 2H), 7.83 (brs, 3H), 8.03 (t, J=6.3 Hz, 1H); ¹³C NMR (125.7 MHz, DMSO-d₆) δ 23.32, 25.99, 26.40, 31.35, 34.23, 34.44, 35.96, 36.27, 39.95, 41.12, 48.25, 54.54, 108.26, 110.76, 121.79, 127.45, 142.86, 149.35, 155.54. Anal. Calcd for C₂₈H₄₂N₂O₈S: C, 59.34; H, 7.47; N, 4.94. Found: C, 59.12; H, 7.60; N, 4.80.

cis-Adamantane-2-spiro-3′-8′-[4′-[(1′-piperazinylcarbonyl)oxy]phenyl]-1′,2′,4′-trioxaspiro[4.5]decane mesylate (OZ414). To a solution of the p-nitrophenyl carbonate (0.865 g, 1.7 mmol, see OZ413) in CH₂Cl₂ (15 ml) was added a solution of piperazine (0.72 g, 8.5 mmol) in CH₂Cl₂ (5 ml). The reaction mixture was stirred at rt overnight and then quenched with water (15 ml). After separation of the organic layer, the aqueous layer was extracted with CH₂Cl₂ (2×15 ml). The combined extracts were washed with water (3×25 ml), dried over MgSO₄, concentrated to afford the free base. To the solution of the above free base in CH₂Cl₂ (5 ml) at 0° C. was added a solution of methanesulfonic acid (0.14 g, 1.44 mmol) in ether (5 ml). The solid obtained was filtered, washed with ether (20 ml), and dried to afford trioxolane OZ414 (0.67 g, 70%) as a colorless solid. mp 158-160° C.; ¹H NMR (500 MHz, CDCl₃) δ 1.62-2.11 (m, 22H), 2.59-2.68 (m, 1H), 2.82 (s, 3H), 3.32 (brs, 4H), 3.87 (brs, 2H), 3.97 (brs, 2H), 7.01 (d, J=8.3 Hz, 2H), 7.20 (d, J=8.3 Hz, 2H), 9.24 (brs, 2H); ¹³C NMR (125.7 MHz, CDCl₃) δ 26.44, 26.84, 31.44, 34.61, 34.76, 36.36, 36.75, 39.42, 42.30, 43.35, 108.24, 111.38, 121.28, 127.69, 143.69, 148.97, 153.30. Anal. Calcd for C₂₈H₄₀N₂O₈S: C, 59.55; H, 7.14; N, 4.96. Found: C, 59.68; H, 7.33; N, 4.85.

Bicyclo[3.3.1]nonane-9-spiro-3′-1′,2′,4′-trioxaspiro[4.5]decane (OZ415). A solution of O-methyl cyclohexanone oxime (0.50 g, 3.94 mmol) and bicyclo[3.3.1]nonan-9-one (0.50 g, 3.62 mmol) in cyclohexane (90 ml) and CH₂Cl₂ (30 ml) was treated with ozone according to the general procedure. After removal of solvents, the crude product was purified by chromatography (silica gel, 2% ether in hexanes) to afford trioxolane OZ415 (0.08 g, 9%) as a colorless solid. mp 50-52° C.; ¹H NMR (500 MHz, CDCl₃) δ 1.28-2.13 (m, 24H); ¹³C NMR (125.7 MHz, CDCl₃) δ 20.50, 20.93, 23.89, 25.00, 29.36, 29.67, 34.88, 36.31, 108.84, 109.77. Anal. Calcd for C₁₅H₂₄O₃: C, 71.39; H, 9.59. Found: C, 71.60; H, 9.67.

(cis-Adamantane-2-spiro-3′-1′,2′,4′-trioxaspiro[4.5]decan-8′-yl)methyl sodium sulfate (OZ416). To a stirred suspension of sulfur trioxide pyridine complex (1.62 g, 10 mmol) in dry pyridine (5 ml) was added OZ119 (1.0 g, 3.4 mmol) in portions. Stirring was continued at rt for 24 h. After removal of the pyridine under reduced pressure, saturated aq. Na₂CO₃ solution (10 ml) was added. The mixture was kept at −20° C. for 4 h and then at 0° C. overnight. The precipitate was filtered, washed with cold water (5 ml), and dried in a vacuum oven to give trioxolane OZ416 (0.55 g, 41%) as a colorless solid. mp 156-157° C.; ¹H NMR (500 MHz, DMSO-d₆) δ 1.01-1.12 (m, 2H), 1.51-1.95 (m, 21H), 3.52 (d, J=6.3 Hz, 2H); ¹³C NMR (125.7 MHz, DMSO-d₆) δ 26.01, 26.41, 26.68, 33.39, 34.44, 34.46, 35.64, 35.96, 36.29, 69.96, 108.80, 110.60. Anal. Calcd for C₁₇H₂₅NaO₇S: C, 51.51; H, 6.36. Found: C, 51.47; H, 6.31.

cis-Adamantane-2-spiro-3′-8′-[4′-[(ethoxycarbonyl)methoxy]phenyl]-1′,2′,4′-trioxaspiro[4.5]decane (OZ417). A mixture of OZ288 (0.50 g, 1.4 mmol), ethyl α-bromoacetate (0.26 g, 1.5 mmol), and anhydrous potassium carbonate (0.39 g, 2.8 mmol) in acetone (50 ml) was gently refluxed overnight. The reaction mixture was cooled to rt, filtered to remove the solid, and concentrated. The residue was dissolved in CH₂Cl₂ (30 ml), washed with water (2×20 ml) and brine (20 ml), dried over MgSO₄, filtered, and concentrated to afford trioxolane OZ417 (0.60 g, 97%) as a colorless solid. mp 124-126° C.; ¹H NMR (500 MHz, CDCl₃) δ 1.29 (t, J=7.1 Hz, 3H), 1.61-2.09 (m, 22H), 2.42-2.56 (m, 1H), 4.27 (q, J=7.1 Hz, 2H), 4.59 (s, 2H), 6.83 (d, J=8.3 Hz, 2H), 7.12 (d, J=8.8 Hz, 2H); ¹³C NMR (125.7 MHz, CDCl₃) δ 14.15, 26.49, 26.89, 31.58, 34.72, 34.80, 36.41, 36.81, 42.06, 61.31, 65.59, 108.40, 111.38, 114.59, 127.73, 139.47, 156.22, 169.10. Anal. Calcd for C₂₆H₃₄O₆: C, 70.56; H, 7.74. Found: C, 70.37; H, 7.90.

cis-Adamantane-2-spiro-3′-8′-[4′-(carboxymethoxy)phenyl]-1′,2′,4′-trioxaspiro[4.5]decane (OZ418). To a solution of OZ417 (0.52 g, 1.18 mmol) in MeOH (20 ml) and THF (20 ml) was added 15% aq. KOH solution (1 ml). The resulting mixture was stirred at 50° C. for 4 h. After the solvents were removed, the residue was diluted with water (10 ml) and acidified with acetic acid (5 ml). The precipitate was collected by filtration, washed with cold water, and dried in a vacuum oven at 40° C. to afford trioxolane OZ418 (0.49 g, 100%) as a colorless solid. mp 156-157° C.; ¹H NMR (500 MHz, CDCl₃) δ 1.61-2.09 (m, 22H), 2.44-2.58 (m, 1H), 4.64 (s, 2H), 6.86 (d, J=8.3 Hz, 2H), 7.15 (d, J=8.8 Hz, 2H); ¹³C NMR (125.7 MHz, CDCl₃) δ 26.48, 26.88, 31.57, 34.69, 34.80, 36.41, 36.80, 42.05, 65.00, 108.36, 111.41, 114.59, 127.89, 139.97, 155.69, 172.99. Anal. Calcd for C₂₄H₃₀O₆: C, 69.54; H, 7.30. Found: C, 69.68; H, 7.25.

cis-Adamantane-2-spiro-3′-8′-[[[(1′,1′-dioxido-4′-thiomorpholinyl)acetyl]amino]methyl]-1′,2′,4′-trioxaspiro[4.5]decane p-tosylate (OZ419). To a solution of thiomorpholinoacetic acid 1,1-dioxide monohydrate (232 mg, 1.1 mmol), EDCI (216 mg, 1.1 mmol), and HOBt (152 mg, 1.1 mmol) in DMF (30 ml) was added a solution of OZ209 (161 mg, 0.55 mmol) and TEA (0.1 mL, 0.71 mmol) in DMF (5 ml). After stirring at rt for 48 h, the reaction mixture was poured into ice-water (100 ml). The precipitate was collected by filtration, washed with ice-water, and dried under vacuum to give the free base. To a solution of the above free base was added a solution of p-toluenesulfonic acid monohydrate (85 mg, 0.45 mmol) in ethanol (10 ml). The mixture was stirred for 1 h and then mixed with ether (20 ml). The precipitate was collected by filtration, washed with ether, and dried under vacuum to give trioxolane OZ419 (265 mg, 75%) as a brownish powder. mp 158-160° C.; ¹H NMR (500 MHz, DMSO-d₆) δ 0.98-1.17 (m, 2H), 1.41-2.01 (m, 21H), 2.29 (s, 3H), 3.01 (s, 2H), 3.52 (s, 4H), 3.63 (s, 4H), 3.97 (s, 2H), 7.12 (d, J=6.8 Hz, 2H), 7.49 (d, J=7.3 Hz, 2H), 8.49 (brs, 1H); ¹³C NMR (125.7 MHz, DMSO-d₆) δ 20.96, 25.98, 26.39, 27.55, 33.36, 34.43, 35.71, 35.93, 36.25, 43.95, 48.41, 51.18, 56.61, 108.65, 110.70, 125.68, 128.26, 137.86, 145.80, 164.95. Anal. Calcd for C₃₀H₄₄N₂O₉S₂: C, 56.23; H, 6.92; N, 4.37. Found: C, 56.12; H, 7.07; N, 4.25.

cis-Adamantane-2-spiro-3′-8′-(1′-piperazinylmethyl)-1′,2′,4′-trioxaspiro[4.5]decane di-p-tosylate (OZ420). Step 1. A mixture of 1-Boc-piperazine (2.30 g, 12.0 mmol), MeOH (50 ml), HOAc (0.7 ml, 12.0 mmol), and OZ390 (1.76 g, 6.0 mmol) was stirred at rt for 1 h before NaBH₃CN (795 mg, 12.0 mmol) was added. The resulting suspension was stirred at rt for 24 h and then quenched with saturated aq. Na₂CO₃ (120 ml, final pH=10). The solution was concentrated, mixed with water (100 ml), and extracted with CH₂Cl₂ (3×60 ml). The organic layers were combined, washed with water (3×40 ml), dried over MgSO₄ and evaporated to give the Boc-protected piperazine (2.4 g, 86%). ¹H NMR (500 MHz, CDCl₃) δ 1.13-1.19 (m, 2H), 1.45 (s, 9H), 1.62-2.00 (m, 21H), 2.13 (d, J=7.3 Hz, 2H), 2.32 (t, J=4.6 Hz, 4H), 3.40 (t, J=4.6 Hz, 4H). Step 2. To a solution of p-toluensulfonic acid monohydrate (7.7 g, 40 mmol) in CH₂Cl₂ (8 ml) and THF (15 ml) was added the above Boc-protected piperazine (1.2 g, 2.6 mmol). After the mixture was stirred at rt for 2.5 h, the resulting precipitate was collected by filtration. After the solid salt was basified with 0.5 M aq. NaOH to pH=11, the alkaline solution was extracted with CH₂Cl₂ (3×20 ml). The combined organic layers were dried over MgSO₄ and evaporated to give the piperazino free base (0.45 g, 48%) as a wax-like solid. ¹H NMR (500 MHz, CDCl₃) δ 1.11-1.19 (m, 2H), 1.62-2.00 (m, 22H), 2.11 (d, J=7.3 Hz, 2H), 2.35 (brs, 4H), 2.87 (t, J=4.8 Hz, 4H). Step 3. The piperazino free base (200 mg, 0.55 mmol) was added to a solution of p-toluenesulfonic acid monohydrate (220 mg, 1.15 mmol) in ether (30 mL). After evaporation, the residue was washed with ether to give trioxolane OZ420 as a brownish powder (250 mg, 64%). mp 155-157° C.; ¹H NMR (500 MHz, DMSO-d₆) δ 1.01-1.19 (m, 2H), 1.59-1.99 (m, 21H), 2.31 (s, 6H), 3.05 (d, J=4.4 Hz, 2H), 3.24-3.78 (m, 8H), 5.72 (s, 1H), 7.17 (d, J=7.8 Hz, 4H), 7.53 (d, J=7.8 Hz, 4H), 9.05 (brs, 2H); ¹³C NMR (125.7 MHz, DMSO-d₆) δ 21.18, 26.19, 26.59, 27.53, 30.41, 33.07, 34.64, 36.14, 36.43, 40.50, 48.95, 55.23, 61.12, 108.35, 111.15, 125.89, 128.70, 138.71, 145.05. Anal. Calcd for C₃₅H₅₀N₂O₉S₂: C, 59.47; H, 7.13; N, 3.96. Found: C, 59.24; H, 7.30; N, 3.74.

N-[cis-Adamantane-2-spiro-3′-1′,2′,4′-trioxaspiro[4.5]decane-8′-methyl]-L-2-phenylglycinamide p-tosylate (OZ421). Step 1. In a 50 ml flask fitted with a reflux condenser (capped with a CaCl₂ drying tube) were placed L(+)-phenylglycine (3.02 g, 20 mmol), KOH (1.45 g, 22 mmol), and absolute ethanol (15 ml). The mixture was heated until the solution became clear. A solution of ethyl acetoacetate (3.0 g, 23 mmol) in ethanol (6 ml) was added. The reaction mixture was refluxed for 1 h, cooled to rt, and concentrated to about 10 ml. The precipitate was collected by filtration and washed with cold ethanol to give potassium L(+)-phenylglycine Dane salt (4.7 g, 85%) as a white powder. ¹H NMR (500 MHz, DMSO-d₆) δ 1.16 (t, J=7.0 Hz, 3H), 1.60 (s, 3H), 3.99 (q, J=7.0 Hz, 2H), 4.23 (s, 1H), 4.67 (d, J=6.4 Hz, 1H), 7.15-7.26 (m, 5H) and 9.56 (d, J=6.4 Hz, 1H). Step 2. In a 25-ml three-necked flask, equipped with a thermometer, a CaCl₂ drying tube and an argon inlet, were placed potassium L(+)-phenylglycine Dane salt (1.0 g, 5 mmol), CH₂Cl₂ (10 ml), DMF (2 ml), and 2-picoline (40 mg). After the mixture was cooled to −30° C. on a dry ice-ethanol bath, trimethylacetyl chloride (0.68 ml, 5.5 mmol) was added to the solution. The suspension was stirred at −25° C. for 30 min and then cooled to −60° C. After a solution of OZ209 (0.50 g, 1.7 mmol) in CH₂Cl₂ (5 ml) was added, the mixture was stirred overnight. Under ice-water cooling, the reaction was quenched with 1 M aq. HCl (pH=1). After the solution was stirred for 20 min, the CH₂Cl₂ layer was separated and washed with 1 M aq HCl (2×5 ml). The combined aqueous layers were basified with NH₄OH to pH=9 and extracted with CH₂Cl₂ (3×20 ml). The organic layers were combined, dried over MgSO₄, and evaporated to dryness. The residue was purified by chromatography (silica gel, 80% ether in hexanes) to give the free base (290 mg, 40%). A mixture of the free base and p-toluenesulfonic acid monohydrate (135 mg, 0.70 mmol) in CH₂Cl₂ (20 ml) was stirred at rt for 1 h and then mixed with ether (20 ml). The resulting precipitate was collected by filtration, washed with ether, and dried to give trioxolane OZ421 (280 mg, 28%) as a yellow powder. mp 105-107° C.; ¹H NMR (500 MHz, CD₃OD) δ 1.01-1.16 (m, 2H), 1.42-2.05 (m, 21H), 2.37 (s, 3H), 2.95-3.05 (m, 1H), 3.11-3.17 (m, 1H), 4.93 (s, 1H), 7.23 (d, J=8.3 Hz, 2H), 7.41-7.56 (m, 5H), 7.70 (d, J=8.3 Hz, 2H); ¹³C NMR (125.7 MHz, CD₃OD) δ 21.31, 27.95, 28.35, 28.60, 28.67, 34.69, 35.76, 37.38, 37.84, 57.89, 109.73, 112.24, 126.97, 129.16, 129.82, 130.48, 131.09, 134.72, 141.70, 143.52, 168.94. Anal. Calcd for C₃₂H₄₂N₂O₇S: C, 64.19; H, 7.07; N, 4.68. Found: C, 63.96; H, 6.99; N, 4.55.

N-[cis-Adamantane-2-spiro-3′-1′,2′,4′-trioxaspiro[4.5]decane-8′-methyl]-L-2-phenylglycyl-L-2-phenylglycinamide p-tosylate (OZ422). In a 25-ml three-necked flask, equipped with a thermometer, a CaCl₂ drying tube and an argon inlet, were placed potassium L(+)-phenylglycine Dane salt (1.0 g, 5.0 mmol), CH₂Cl₂ (10 ml), DMF (2 ml) and 2-picoline (40 mg). After the mixture was cooled to −30° C. on a dry ice-ethanol bath, trimethylacetyl chloride (0.68 ml, 5.5 mmol) was added to the solution. The suspension was stirred at −25° C. for 30 min and then cooled to −60° C. After a solution of the OZ421 free base (300 mg, 0.70 mmol) in CH₂Cl₂ (5 ml) was added, the mixture was stirred overnight. Under ice-water cooling, the reaction was quenched with 1 M aq. HCl (pH=1). After the solution was stirred for 20 min, the CH₂Cl₂ layer was separated and washed with 1 M aq HCl (2×5 ml). The combined aqueous layers were basified with NH₄OH to pH=9 and extracted with CH₂Cl₂ (3×20 ml). The organic layers were combined, dried over MgSO₄, and evaporated to dryness. The residue was purified by chromatography (silica gel, 80% ether in hexanes) to give the free base (190 mg, 49%). A mixture of the free base and p-toluenesulfonic acid monohydrate (65 mg, 0.34 mmol) in CH₂Cl₂ (20 ml) was stirred at rt for 1 h and then mixed with ether (20 ml). The resulting precipitate was collected by filtration, washed with ether, and dried to give trioxolane OZ422 (200 mg, 39%) as a yellow powder. mp 138-140° C.; ¹H NMR (500 MHz, CD₃OD) δ 0.97-1.12 (m, 2H), 1.34-2.05 (m, 21H), 2.36 (s, 3H), 2.86-2.93 (m, 1H), 2.95-3.11 (m, 1H), 5.09 (s, 1H), 5.46 (s, 1H), 7.22 (d, J=7.8 Hz, 2H), 7.29-7.59 (m, 10H), 7.70 (d, J=8.3 Hz, 2H); ¹³C NMR (125.7 MHz, CD₃OD) δ 21.31, 27.94, 28.35, 28.58, 34.73, 35.76, 37.40, 37.80, 37.85, 45.54, 57.58, 58.96, 109.78, 112.20, 126.97, 128.64, 129.32, 129.48, 129.79, 129.81, 130.48, 131.06, 134.36, 138.64, 141.66, 143.53, 168.22, 171.59. Anal. Calcd for C₄₀H₄₉N₃O₈S: C, 65.64; H, 6.75; N, 5.74. Found: C, 65.46; H, 6.88; N, 5.76.

N-[cis-Adamantane-2-spiro-3′-1′,2′,4′-trioxaspiro[4.5]decane-8′-methyl]-D-2-phenylglycinamide p-tosylate (OZ423). Step 1. In a 50 ml flask fitted with a reflux condenser (capped with a CaCl₂ drying tube) were placed D(−)-phenylglycine (3.02 g, 20 mmol), KOH (1.45 g, 22 mmol), and absolute ethanol (15 ml). The mixture was heated until the solution became clear. A solution of ethyl acetoacetate (3.0 g, 23 mmol) in ethanol (6 ml) was added. The reaction mixture was refluxed for 1 h, cooled to rt, and concentrated to about 10 ml. The precipitate was collected by filtration and washed with cold ethanol to give potassium D(−)-phenylglycine Dane salt (4.4 g, 80%) as a white powder. ¹H NMR (500 MHz, DMSO-d₆) δ 1.16 (t, J=7.0 Hz, 3H), 1.60 (s, 3H), 3.99 (q, J=7.0 Hz, 2H), 4.23 (s, 1H), 4.67 (d, J=6.4 Hz, 1H), 7.15-7.26 (m, 5H) and 9.56 (d, J=6.4 Hz, 1H). Step 2. In a 25-ml three-necked flask, equipped with a thermometer, a CaCl₂ drying tube and an argon inlet, were placed potassium D(−)-phenylglycine Dane salt (1.0 g, 5.0 mmol), CH₂Cl₂ (10 ml), DMF (2 ml), and 2-picoline (40 mg). After the mixture was cooled to −30° C. on a dry ice-ethanol bath, trimethylacetyl chloride (0.68 ml, 5.5 mmol) was added to the solution. The suspension was stirred at −25° C. for 30 min and then cooled to −60° C. After a solution of OZ209 (0.50 g, 1.7 mmol) in CH₂Cl₂ (5 ml) was added, the mixture was stirred overnight. Under ice-water cooling, the reaction was quenched with 1 M aq. HCl (pH=1). After the solution was stirred for 20 min, the CH₂Cl₂ layer was separated and washed with 1 M aq HC1 (2×5 ml). The combined aqueous layers were basified with NH₄H to pH=9 and extracted with CH₂Cl₂ (3×20 ml). The organic layers were combined, dried over MgSO₄, and evaporated to dryness. The residue was purified by chromatography (silica gel, 80% ether in hexanes) to give the free base (310 mg, 43%). A mixture of the free base and p-toluenesulfonic acid monohydrate (145 mg, 0.75 mmol) in CH₂Cl₂ (20 ml) was stirred at rt for 1 h and then mixed with ether (20 ml). The resulting precipitate was collected by filtration, washed with ether, and dried to give trioxolane OZ423 (320 mg, 31%) as a yellow powder. mp 137-139° C.; ¹H NMR (500 MHz, CD₃OD) δ 1.01-1.16 (m, 2H), 1.42-2.05 (m, 21H), 2.38 (s, 3H), 3.01 (brs, 1H), 3.11 (brs, 1H), 4.95 (s, 1H), 7.24 (brs, 2H), 7.41-7.56 (m, 5H), 7.71 (brs, 2H); ¹³C NMR (125.7 MHz, CD₃OD) δ 21.32, 27.94, 28.34, 28.60, 28.67, 34.69, 35.76, 37.37, 37.83, 45.81, 57.87, 109.73, 112.22, 126.97, 129.16, 129.82, 130.46, 131.06, 134.73, 141.70, 143.53, 168.95. Anal. Calcd for C₃₂H₄₂N₂O₇S: C, 64.19; H, 7.07; N, 4.68. Found: C, 64.06; H, 7.04; N, 4.87.

cis-Adamantane-2-spiro-3′-8′-[4′-[(1′-piperazinylcarbonyl)methoxy]phenyl]-1′,2′,4′-trioxaspiro[4.5]decane p-tosylate (OZ424). Step 1. A solution of OZ418 (0.40 g, 0.97 mmol), HOSu (0.13 g, 1.1 mmol), and EDCI (0.24 g, 1.2 mmol) in DMF (10 ml) was stirred at rt for 24 h. Under ice cooling, the reaction was quenched with water (30 ml). The precipitate was collected by filtration, washed with cold water, and dried in a vacuum oven at 40° C. to afford the desired active ester (0.45 g, 92%) as a colorless solid. mp 145-146° C.; ¹H NMR (500 MHz, CDCl₃) δ 1.66-2.08 (m, 22H), 2.46-2.56 (m, 1H), 2.86 (s, 4H), 4.94 (s, 2H), 6.88 (d, J=8.5 Hz, 2H), 7.15 6.88 (d, J=8.5 Hz, 2H). Step 2. To a solution of piperazine (0.05 g, 0.57 mmol) in CHCl₃ (10 ml) was added dropwise a solution of the active ester (0.10 g, 0.20 mmol) in CHCl₃ (10 ml). The resulting mixture was stirred at rt for 4 h and then quenched with water (10 ml). After separation of the organic layer, the aqueous layer was extracted with CHCl₃ (2×20 ml). The combined extracts were washed with water (2×10 ml) and brine (10 ml), dried over MgSO₄, filtered, and concentrated. The residue was dissolved in ether (10 ml) and then a solution of p-toluenesulfonic acid monohydrate (0.04 g) in ether (10 ml) was added. The precipitate was collected by filtration to afford trioxolane OZ424 (0.09 g, 69%) as a colorless solid. mp 130-131° C.; ¹H NMR (500 MHz, DMSO-d₆) δ 1.45-1.62 (m, 2H), 1.65-1.95 (m, 20H), 2.29 (s, 3H), 2.49-2.61 (m, 1H), 3.10 (brs, 2H), 3.16 (brs, 2H), 3.65 (s, 4H), 4.82 (s, 2H), 6.85 (d, J=8.8 Hz, 2H), 7.11 (d, J=7.8 Hz, 2H), 7.12 (d, J=8.8 Hz, 2H), 7.47 (d, J=8.3 Hz, 2H), 8.70 (brs, 2H); ¹³C NMR (125.7 MHz, DMSO-d₆) δ 20.95, 25.98, 26.39, 31.47, 34.26, 34.43, 35.95, 36.25, 39.79, 39.96, 40.87, 42.98 (br), 65.91, 108.31, 110.73, 114.70, 125.66, 127.52, 128.19, 137.68, 138.63, 146.02, 156.40, 166.48. Anal. Calcd for C₃₅H₄₆N₂O₈S: C, 64.20; H, 7.08; N, 4.28. Found: C, 63.97; H, 6.89; N, 4.27.

cis-Adamantane-2-spiro-3′-8′-[4′-[[[(2′-amino-2′-methylpropyl)amino]carbonyl]methoxy]phenyl]-1′,2′,4′-trioxaspiro[4.5]decane p-tosylate (OZ425). To a solution of 1,2-diamino-2-methylpropane (0.20 g, 2.2 mmol) in CHCl₃ (10 ml) was added dropwise the solution of the OZ418-HOSu active ester (0.30 g, 0.60 mmol, see OZ424) in CHCl₃ (20 ml). The resulting mixture was stirred at rt for 4 h and then quenched with water (10 ml). After separation of the organic layer, the aqueous layer was extracted with CHCl₃ (2×20 ml). The combined extracts were washed with water (2×10 ml) and brine (10 ml), dried over MgSO₄, filtered, and concentrated. The residue was dissolved in ether (10 ml) and then the solution of p-toluenesulfonic acid monohydrate (0.08 g) in ether (10 ml) was added. The precipitate was collected by filtration to afford trioxolane OZ425 (0.18 g, 46%) as a colorless solid. mp 118-120° C.; ¹H NMR (500 MHz, DMSO-d₆) δ 1.17 (s, 6H), 1.45-1.61 (m, 2H), 1.64-1.95 (m, 20H), 2.29 (s, 3H), 2.49-2.62 (m, 1H), 3.27 (d, J=6.3 Hz, 2H), 4.51 (s, 2H), 6.91 (d, J=8.8 Hz, 2H), 7.11 (d, J=7.8 Hz, 2H), 7.15 (d, J=8.8 Hz, 2H), 7.47 (d, J=8.3 Hz, 2H), 7.70 (brs, 3H), 8.32 (t, J=6.3 Hz, 1H); ¹³C NMR (125.7 MHz, DMSO-d₆) δ 20.95, 23.53, 25.98, 26.39, 31.46, 34.26, 34.44, 35.96, 36.26, 39.96, 40.87, 45.87, 54.83, 67.26, 108.30, 110.74, 114.88, 125.67, 127.66, 128.19, 137.73, 138.95, 145.98, 156.25, 169.41.

cis-Adamantane-2-spiro-3′-8′-[[(1′-piperazinylacetyl)amino]methyl]-1′,2′,4′-trioxaspiro[4.5]decane di-p-tosylate (OZ426). Step 1. To a solution of OZ209 (7.00 g, 239 mmol) in CH₂Cl₂ (60 ml) at 0° C. was added 8% aq. NaOH (28 ml) dropwise. After the mixture was cooled to −10° C. using an ice-salt (NaCl) bath, bromoacetyl chloride (2.13 ml, 256 mmol) was added dropwise. The reaction mixture was left to warm up to rt overnight and then quenched with water (20 ml). The organic layer was separated, washed with water (20 ml), dried with MgSO₄, and concentrated to afford the 2-bromoacetamide intermediate (9.90 g, 100%) as a brown solid. ¹H NMR (500 MHz, CDCl₃) δ 1.23-1.26 (m, 2H), 1.57-1.99 (m, 21H), 3.16 (t, J=6.4 Hz, 2H), 3.89 (s, 2H), 6.55 (brs, 1H). Step 2. To a solution of piperazine (0.29 ml, 2.9 mmol) in acetonitrile (10 ml) at 0° C. was added dropwise a solution of the above 2-bromoacetamide intermediate (0.29 g, 0.724 mmol) in acetonitrile (10 ml). After the addition, the reaction was stirred at rt for 2 h. The solution was filtered, concentrated, and diluted with CH₂Cl₂ (20 ml). The organic layer was washed with water (2×10 ml) and brine (10 ml), dried with MgSO₄, and concentrated to afford the desired free base (0.22 g, 72%) as a brown oil. ¹H NMR (500 MHz, CDCl₃) δ 1.23-1.26 (m, 2H), 1.57-1.99 (m, 21H), 2.50 (brs, 4H), 2.89-2.92 (m, 5H), 2.98 (s, 2H), 3.16 (t, J=6.4 Hz, 2H), 7.27 (brs, 1H). To a solution of the above free base in EtOAc (5 ml) was added p-toluenesulfonic acid monohydrate (199 mg, 1.05 mmol) in ethanol (3 ml). The precipitate was filtered, washed with ether, and dried to give trioxolane OZ426 (0.105 g, 26%) as a white solid. mp 155-156° C.; ¹H NMR (500 MHz, DMSO-d₆+D₂O) δ 1.01-1.12 (m, 2H), 1.45-1.93 (m, 21H), 2.31 (s, 6H), 2.89 (brs, 4H), 2.99 (d, J=6.8 Hz, 2H), 3.21 (brs, 4H), 3.32 (brs, 2H), 7.17 (d, J=7.8 Hz, 4H), 7.52 (d, J=7.8 Hz, 4H); ¹³C NMR (125.7 MHz, DMSO-d₆+D₂O) δ 21.42, 26.42, 26.82, 28.00, 33.87, 34.88, 36.18, 36.41, 36.66, 39.95, 42.62, 44.10, 49.65, 109.26, 111.35, 126.14, 128.97, 139.13, 145.17, 166.52. Anal. Calcd for C₃₇H₅₃N₃O₁₀S₂: C, 58.17; H, 6.99; N, 5.50. Found: C, 57.99; H, 6.88; N, 5.38.

cis-Adamantane-2-spiro-3′-8′-[[[(cyclopropylamino)acetyl]amino]methyl]-1′,2′,4′-trioxaspiro[4.5]decane p-tosylate (OZ427). To a solution of cyclopropylamine (0.415 ml, 6.0 mmol) in acetonitrile (10 ml) at 0° C. was added dropwise a solution of the above 2-bromoacetamide intermediate (0.414 g, 1.0 mmol, see OZ426) in acetonitrile (10 ml). After the addition, the reaction was stirred at rt for 2 h. The solution was concentrated and diluted with CH₂Cl₂ (20 ml). The organic layer was extracted with water (2×10 ml) and brine (10 ml) and dried with MgSO₄. The solution was concentrated and diluted with ethyl acetate (10 ml). After the resulting solid was filtered off, the filtrate was concentrated to afford the desired free base (0.254 g, 65%) as a brown oil. ¹H NMR (500 MHz, CDCl₃) δ 0.49-0.54 (m, 4H), 1.23-1.26 (m, 3H), 1.57-1.99 (m, 22H), 3.13 (t, J=6.3 Hz, 2H), 3.46 (s, 2H), 7.16 (brs, 1H). To a solution of the above free base (0.254 g, 0.651 mmol) in EtOAc (10 ml) was added p-toluenesulfonic acid monohydrate (0.124 g, 0.651 mmol) in ethanol (2 ml). After removal of the solvent, the residue was crystallized from CH₂Cl₂-ether (2:3) to give trioxolane OZ427 (0.206 g, 52%) as a white solid. mp 133-134° C.; ¹H NMR (500 MHz, CDCl₃+DMSO-d₆) δ 0.68-0.79 (m, 2H), 1.01-1.19 (m, 4H), 1.41-2.03 (m, 21H), 2.36 (s, 3H), 2.67-2.80 (m, 1H), 3.05 (t, J=6.4 Hz, 2H), 3.93 (s, 2H), 7.17 (d, J=8.3 Hz, 2H), 7.73 (d, J=7.8 Hz, 2H), 8.33 (t, J=5.7 Hz, 1H), 9.09 (brs, 2H); ¹³C NMR (125.7 MHz, CDCl₃+DMSO-d₆) δ 3.27, 20.80, 25.93, 26.32, 27.25, 30.28, 33.12, 34.26, 35.41, 35.85, 36.24, 44.29, 49.09, 108.16, 110.66, 125.42, 128.21, 139.25, 142.37, 164.46. Anal. Calcd for C₂₉H₄₂N₂O₇S: C, 61.90; H, 7.52; N, 4.98. Found: C, 61.79; H, 7.57; N, 4.91.

cis-Adamantane-2-spiro-3′-8′-[[[[(cyclopropylmethyl)amino]acetyl]amino]methyl]-1′,2′,4′-trioxaspiro[4.5]decane p-tosylate (OZ428). To a solution of (aminomethyl)cyclopropane (0.347 ml, 4.0 mmol) in acetonitrile (10 ml) at 0° C. was added dropwise a solution of the above 2-bromoacetamide intermediate (0.414 g, 1.0 mmol, see OZ426). After the addition, the reaction was stirred at rt for 2 h. The solution was concentrated and diluted with CH₂Cl₂ (20 ml). The organic layer was washed with water (2×10 ml) and brine (10 ml) and dried with MgSO₄. The solution was concentrated to afford the desired free base (0.35 g, 87%) as a brown oil. ¹H NMR (500 MHz, CDCl₃) δ 0.11-0.14 (m, 2H), 0.49-0.51 (m, 2H), 0.90-0.93 (m, 1H), 1.23-1.28 (m, 3H), 1.57-1.99 (m, 20H), 2.45 (d, J=6.8 Hz, 2H), 3.15 (t, J=6.4 Hz, 2H), 3.28 (s, 2H), 7.39 (brs, 1H). To a solution of the above free base in EtOAc (5 ml) was added a solution of p-toluenesulfonic acid monohydrate (165 mg, 0.865 mmol) in EtOAc (2 ml). After the solution was concentrated, the residue was crystallized from CH₂Cl₂ (5 ml) to give trioxolane OZ428 (0.278 g, 53%) as a white solid. mp 152-153° C.; ¹H NMR (500 MHz, CDCl₃) δ 0.29-0.38 (m, 2H), 0.44-0.63 (m, 2H), 1.01-1.18 (m, 3H), 1.29-2.03 (m, 21H), 2.37 (s, 3H), 2.93 (d, J=7.8 Hz, 2H), 3.00 (t, J=6.1 Hz, 2H), 4.06 (s, 2H), 7.19 (d, J=7.8 Hz, 2H), 7.72 (d, J=8.3 Hz, 2H), 8.16 (t, J=5.7 Hz, 1H), 8.46 (brs, 2H); ¹³C NMR (125.7 MHz, CDCl₃) δ 4.25, 7.43, 21.29, 26.47, 26.87, 27.71, 33.55, 34.76, 35.82, 36.37, 36.79, 45.11, 48.16, 53.44, 108.58, 111.17, 125.83, 129.00, 140.59, 141.67, 165.19. Anal. Calcd for C₃₀H₄₄N₂O₇S: C, 62.47; H, 7.69; N, 4.86. Found: C, 62.60; H, 7.70; N, 4.96.

cis-Adamantane-2-spiro-3′-8′-[[(4′-morpholinylacetyl)amino]methyl]-1′,2′,4′-trioxaspiro[4.5]decane maleate (OZ430). To a solution of morpholine (0.35 ml, 4.0 mmol) in acetonitrile (10 ml) at 0° C. was added dropwise a solution of the 2-bromoacetamide intermediate (0.414 g, 1.0 mmol, see OZ426) in acetonitrile (10 ml). After the addition, the reaction was stirred at rt for 3 h. After filtration, the filtrate was concentrated and dissolved in CH₂Cl₂ (20 ml). The resulting solution was washed with water (3×10 ml) and dried with MgSO₄. The solution was concentrated to afford the desired free base (0.411 g, 98%) as a brown oil. ¹H NMR (500 MHz, CDCl₃) δ 1.23-1.26 (m, 2H), 1.57-1.99 (m, 21H), 2.53 (t, J=4.4, 4H), 3.16 (t, J=6.4 Hz, 2H), 3.71-3.73 (m, 4H), 7.20 (brs, 1H). To a solution of the above oily free base in EtOAc (2 ml) was added a solution of maleic acid (0.114 g, 0.978 mmol) in ethanol (2 ml). After the solution was concentrated, the residue was crystallized from CH₂Cl₂ (3 ml) to give trioxolane OZ430 (0.213 g, 40%) as a white solid. mp 128-131° C.; ¹H NMR (500 MHz, CDCl₃) δ 1.17-1.27 (m, 2H), 1.45-2.03 (m, 21H), 3.14 (t, J=6.2 Hz, 2H), 3.27 (brs, 4H), 3.73 (s, 2H), 3.94 (t, J=4.7 Hz, 4H), 5.49 (brs, 1H), 6.34 (s, 2H), 7.92 (t, J=5.9 Hz, 1H); ¹³C NMR (125.7 MHz, CDCl₃) δ 26.45, 26.84, 27.70, 33.68, 34.77, 36.03, 36.36, 36.76, 44.80, 52.58, 64.46, 108.49, 111.42, 135.39, 169.48.

cis-Adamantane-2-spiro-3′-8′-[[4′-(aminoacetyl)-1′-piperazinyl]methyl]-1′,2′,4′-trioxaspiro[4.5]decane di-p-tosylate (OZ431). To a solution of Boc-Gly-OH (193 mg, 1.1 mmol), EDCI (216 mg, 1.1 mmol), and HOBt (152 mg, 1.1 mmol) in DMF (30 ml) was added a solution of the OZ420 free base (200 mg, 0.55 mmol) and TEA (0.1 ml, 0.71 mmol) in DMF (5 ml). The solution was stirred at rt for 48 h and then poured into ice-water (100 ml). The precipitate was collected by filtration, washed with ice-water, and dried under vacuum. A mixture of the solid precipitate in 1 M ethereal HCl (20 ml) was stirred at rt for 18 h and then evaporated to dryness. The residue was dissolved in water (20 ml), alkalinized with 0.5 M aq. NaOH to pH=12, and extracted with CH₂Cl₂ (3×20 ml). The organic extracts were combined, dried over MgSO₄, evaporated to give the free base crude product (220 mg). A mixture of the free base crude product and p-toluenesulfonic acid monohydrate (200 mg, 1.0 mmol) in CH₂Cl₂ (20 ml) was stirred for 1 h and then treated with ether (20 ml). The resulting precipitate was collected by filtration, washed with ether, and dried under vacuum to give trioxolane OZ431 (250 mg, 60%) as a yellowish powder. mp 146-147° C.; ¹H NMR (500 MHz, DMSO-d₆) δ 1.05-1.19 (m, 2H), 1.61-1.93 (m, 21H), 2.29 (s, 6H), 2.84-2.95 (m, 1H), 2.96-3.09 (m, 3H), 3.10-3.21 (m, 1H), 3.42-3.61 (m, 3H), 3.83-4.06 (m, 3H), 4.34-4.43 (m, 1H), 7.13 (d, J=7.8 Hz, 4H), 7.49 (d, J=7.8 Hz, 4H), 8.07 (brs, 3H), 9.29 (brs, 1H); ¹³C NMR (125.7 MHz, DMSO-d₆) δ 20.96, 25.98, 26.38, 27.55, 30.22, 32.86, 34.42, 34.44, 35.91, 36.23, 38.30, 41.01, 51.10, 51.45, 60.78, 108.12, 110.86, 125.67, 128.30, 137.98, 145.62, 165.15.

cis-Adamantane-2-spiro-3′-8′-[[4′-(2′-amino-2′,2′-dimethylacetyl)-1′-piperazinyl]methyl]-1′,2′,4′-trioxaspiro[4.5]decane di-p-tosylate (OZ432). To a solution of 2-aminoisobutyric acid (116 mg, 1.1 mmol), EDCI (216 mg, 1.1 mmol), and HOBt (152 mg, 1.1 mmol) in DMF (30 ml) was added a solution of the OZ420 free base (200 mg 0.55 mmol) and TEA (0.10 ml, 0.71 mmol) in DMF (5 ml). The reaction mixture was stirred at rt for 48 h and then poured into ice-water (100 ml). The precipitate was collected by filtration, washed with ice-water, and dried under vacuum to give the free base crude product (220 mg). A mixture of the free base crude product and p-toluenesulfonic acid monohydrate (190 mg, 1.0 mmol) in CH₂Cl₂ (20 ml) was stirred for 1 h and then mixed with ether (20 ml). The resulting precipitate was collected by filtration, washed with ether, and dried under vacuum to give trioxolane OZ432 (290 mg, 67%) as a brownish powder. mp 144-146° C.; ¹H NMR (500 MHz, DMSO-d₆) δ 1.09-1.22 (m, 2H), 1.54 (s, 6H), 1.61-1.93 (m, 21H), 2.29 (s, 6H), 2.76-2.82 (m, 1H), 2.95-3.09 (m, 4H), 3.20-3.47 (m, 1H), 3.48-3.60 (m, 2H), 4.23-4.45 (m, 2H), 7.13 (d, J=7.8 Hz, 4H), 7.49 (d, J=7.8 Hz, 4H), 8.17 (brs, 3H), 9.35 (brs, 1H); ¹³C NMR (125.7 MHz, DMSO-d₆) δ 20.96, 22.92, 25.97, 26.37, 27.59, 30.21, 32.87, 34.44, 35.91, 36.23, 51.25, 57.38, 60.65, 108.11, 110.88, 125.66, 128.29, 137.94, 145.68, 169.40.

cis-Adamantane-2-spiro-3′-8′-[4′-(3′-aminopropoxy)phenyl]-1′,2′,4′-trioxaspiro[4.5]decane maleate (OZ433). To a solution of the free base of OZ401 (0.55 g, 1.33 mmol) in CH₂Cl₂ (10 ml) at 0° C. was added dropwise a solution of maleic acid (0.9 eq, 0.14 g, 1.2 mmol) in ether (10 ml). The solid obtained was filtered, washed with ether (25 ml) and dried under vacuum at 40° C. to afford trioxolane OZ433 (0.641 g, 91%) as a colorless solid. mp 145-150° C.; ¹H NMR (500 MHz, DMSO-d₆) δ 1.49-1.62 (m, 2H), 1.63-2.05 (m, 22H), 2.49-2.62 (m, 1H), 2.93 (t, J=7.3 Hz, 2H), 4.01 (t, J=6.1 Hz, 2H), 6.01 (s, 2H), 6.85 (d, J=8.8 Hz, 2H), 7.13 (d, J=8.3 Hz, 2H), 7.17 (brs, 3H); ¹³C NMR (125.7 MHz, DMSO-d₆) δ 25.99, 26.40, 27.34, 31.49, 34.27, 34.43, 35.96, 36.26, 36.73, 40.87, 64.65, 108.31, 110.72, 114.55, 127.62, 136.33, 138.35, 156.75, 167.36. Anal. Calcd for C₂₉H₃₉NO₈: C, 65.77; H, 7.42; N, 2.64. Found: C, 66.05; H, 7.41; N, 2.69.

cis-Adamantane-2-spiro-3′-8′-[4′-(3′-aminopropoxy)phenyl]-1′,2′,4′-trioxaspiro[4.5]decane hydrochloride (OZ434). To a solution of the free base of OZ401 (0.50 g, 1.21 mmol) in CH₂Cl₂ (10 ml) and EtOH (10 ml) at 0° C. was added dropwise 1 M ethereal HCl solution (0.85 ml, 0.85 mmol) diluted with ether (10 ml). The solid obtained was filtered, washed with ether (25 ml), and dried under vacuum at 40° C. to afford OZ434 (0.270 g, 50%) as a colorless solid. mp 105-108° C.; ¹H NMR (500 MHz, DMSO-d₆) δ 1.49-1.62 (m, 2H), 1.63-2.06 (m, 22H), 2.49-2.62 (m, 1H), 2.93 (t, J=7.3 Hz, 2H), 4.02 (t, J=5.9 Hz, 2H), 6.86 (d, J=8.8 Hz, 2H), 7.12 (d, J=8.8 Hz, 2H), 7.89 (brs, 3H); ¹³C NMR (125.7 MHz, DMSO-d₆) δ 25.99, 26.40, 27.06, 31.49, 34.28, 34.43, 35.96, 36.26, 36.38, 40.87, 64.69, 108.31, 110.71, 114.55, 127.60, 138.30, 156.78. Anal. Calcd for C₂₅H₃₆ClNO₄: C, 66.72; H, 8.06; N, 3.11. Found: C, 66.80; H, 7.93; N, 3.14.

cis-Adamantane-2-spiro-3′-8′-[4′-(3′-aminopropoxy)phenyl]-1′,2′,4′-trioxaspiro[4.5]decane p-tosylate (OZ435). To a solution of the free base of OZ401 (0.55 g, 1.3 mmol) in CH₂Cl₂ (10 ml) at 0° C. was added dropwise a solution of p-toluenesulfonic acid monohydrate (0.230 g, 1.2 mmol) in ether (10 ml). The solid obtained was filtered, washed with ether (25 ml), and dried under vacuum at 40° C. to afford trioxolane OZ435 (0.625 g, 78%) as a colorless solid. mp 158-160° C.; ¹H NMR (500 MHz, DMSO-d₆) δ 1.47-1.59 (m, 2H), 1.63-2.06 (m, 22H), 2.29 (s, 3H), 2.50-2.59 (m, 1H), 2.95 (t, J=7.3 Hz, 2H), 4.00 (t, J=6.0 Hz, 2H), 6.85 (d, J=8.8 Hz, 2H), 7.12 (d, J=8.3 Hz, 4H), 7.50 (d, J=8.3 Hz, 2H), 7.73 (brs, 3H); ¹³C NMR (125.7 MHz, DMSO-d₆) δ 20.95, 26.00, 26.41, 27.04, 31.50, 34.28, 34.44, 35.97, 36.27, 36.63, 40.87, 64.61, 108.31, 110.71, 114.55, 125.66, 127.61, 128.28, 137.94, 138.34, 145.66, 156.74. Anal. Calcd for C₃₂H₄₃NO₇S: C, 65.62; H, 7.40; N, 2.39. Found: C, 65.82; H, 7.35; N, 2.49.

cis-Adamantane-2-spiro-3′-8′-[4′-[3′-(1′-piperidinyl)propoxy]phenyl]-1′,2′,4′-trioxaspiro[4.5]decane mesylate (OZ436). To a solution of OZ288 (1.0 g, 2.81 mmol) in dry acetonitrile (50 ml) were added powdered NaOH (0.675 g, 16.85 mmol) and tetrabutylammonium hydrogen sulfate (0.19 g, 0.562 mmol). The mixture was stirred at 25° C. for 30 min before 1-(3-chloropropyl)piperidine hydrochloride (1.67 g, 8.43 mmol) was added. After the addition, it was stirred at 60° C. overnight. The inorganic solid was filtered off and washed with CH₂Cl₂. After removal of the solvents, the residue was dissolved in EtOAc (50 ml). The organic layer was washed with water and brine and dried over MgSO₄. Removal of the solvent afforded the crude free base with some unreacted 1-(3-chloropropyl)piperidine. To the solution of the crude free base in CH₂Cl₂ (10 ml) at 0° C. was added dropwise a solution of methanesulfonic acid (0.25 g, 2.62 mmol) in ether (10 ml). The solid obtained was filtered, washed with ether (25 ml), and dried under vacuum at 40° C. to afford trioxolane OZ436 (0.55 g, 33%) as a colorless solid. mp 144-146° C.; ¹H NMR (500 MHz, CDCl₃) δ 1.35-1.46 (m, 1H), 1.61-2.06 (m, 25H), 2.07-2.19 (m, 2H), 2.31-2.38 (m, 2H), 2.44-2.54 (m, 1H), 2.61-2.70 (m, 2H), 2.78 (s, 3H), 3.17-3.24 (m, 2H), 3.64 (d, J=11.7 Hz, 2H), 4.04 (t, J=5.7 Hz, 2H), 6.77 (d, J=8.8 Hz, 2H), 7.10 (d, J=8.8 Hz, 2H), 10.61 (brs, 1H); ¹³C NMR (125.7 MHz, CDCl₃) δ 22.01, 22.61, 24.04, 26.44, 26.84, 31.57, 34.67, 34.76, 36.37, 36.76, 39.37, 41.97, 53.59, 55.33, 64.79, 108.34, 111.35, 114.22, 127.71, 139.01, 156.52. Anal. Calcd for C₃₁H₄₇NO₇S: C, 64.44; H, 8.20; N, 2.42. Found: C, 64.46; H, 8.08; N, 2.51.

cis-Adamantane-2-spiro-3′-8′-[4′-[2′-(1′-pyrrolidinyl)ethoxy]phenyl]-1′,2′,4′-trioxaspiro[4.5]decane mesylate (OZ437). To a solution of OZ288 (1.0 g, 2.81 mmol) in dry acetonitrile (50 ml) were added powdered NaOH (0.675 g, 16.85 mmol) and tetrabutylammonium hydrogen sulfate (0.19 g, 0.562 mmol). The mixture was stirred at 25° C. for 30 min before 1-(2-chloroethyl)pyrrolidine hydrochloride (1.43 g, 8.43 mmol) was added. After the addition, it was stirred at 60° C. overnight. The inorganic solid was filtered off and washed with CH₂Cl₂. After removal of the solvents, the residue was dissolved in EtOAc (50 ml). The organic layer was washed with water and brine and dried over MgSO₄. Removal of the solvent afforded the free base as a colorless solid. To the solution of the free base in CH₂Cl₂ (10 ml) at 0° C. was added dropwise a solution of methanesulfonic acid (0.20 g, 2.11 mmol) in ether (10 ml). The solid obtained was filtered, washed with ether (25 ml), and dried under vacuum at 40° C. to afford trioxolane OZ437 (1.13 g, 88%) as a colorless solid. mp 142-144° C.; ¹H NMR (500 MHz, CDCl₃) δ 1.61-2.26 (m, 26H), 2.45-2.56 (m, 1H), 2.78 (s, 3H), 2.95-3.11 (m, 2H), 3.52-3.57 (m, 2H), 3.87-3.95 (m, 2H), 4.38-4.44 (m, 2H), 6.84 (d, J=8.8 Hz, 2H), 7.13 (d, J=8.8 Hz, 2H), 11.21 (brs, 1H); ¹³C NMR (125.7 MHz, CDCl₃) δ 23.01, 26.44, 26.83, 31.54, 34.64, 34.75, 36.36, 36.75, 39.20, 41.98, 54.10, 54.75, 63.63, 108.30, 111.37, 114.45, 127.89, 139.75, 155.59. Anal. Calcd for C₂₉H₄₃NO₇S: C, 63.36; H, 7.88; N, 2.55. Found: C, 63.18; H, 7.87; N, 2.61.

cis-Adamantane-2-spiro-3′-8′-[4′-[2′-(1′-piperidinyl)ethoxy]phenyl]-1′,2′,4′-trioxaspiro[4.5]decane mesylate (OZ438). To a solution of OZ288 (1.0 g, 2.81 mmol) in dry acetonitrile (50 ml) were added powdered NaOH (0.675 g, 16.85 mmol) and tetrabutylammonium hydrogen sulfate (0.19 g, 0.562 mmol). The mixture was stirred at 25° C. for 30 min before 1-(2-chloroethyl)piperidine hydrochloride (1.55 g, 8.43 mmol) was added. After the addition, it was stirred at 60° C. overnight. The inorganic solid was filtered off and washed with CH₂Cl₂. After removal of the solvents, the residue was dissolved in EtOAC (50 ml). The organic layer was washed with water and brine and dried over MgSO₄. Removal of the solvent afforded the free base. To a solution of the free base in CH₂Cl₂ (10 ml) at 0° C. was added dropwise a solution of methanesulfonic acid (0.115 g, 1.19 mmol) in ether (10 ml). The resulting solid was filtered, washed with ether (25 ml), and dried under vacuum at 40° C. to afford trioxolane OZ438 (0.637 g, 85%) as a colorless solid. mp 135-137° C.; ¹H NMR (500 MHz, CDCl₃) δ 1.37-1.49 (m, 1H), 1.61-2.19 (m, 27H), 2.44-2.56 (m, 1H), 2.80 (s, 3H), 2.78-2.93 (m, 2H), 3.48-3.51 (m, 2H), 3.71 (d, J=2.2 Hz, 2H), 4.41-4.46 (m, 2H), 6.83 (d, J=8.3 Hz, 2H), 7.13 (d, J=8.3 Hz, 2H), 10.78 (brs, 1H); ¹³C NMR (125.7 MHz, CDCl₃) δ 21.76, 22.79, 26.43, 26.83, 31.54, 34.63, 34.75, 36.36, 36.74, 39.32, 41.97, 54.06, 56.25, 62.88, 108.29, 111.37, 114.41, 127.88, 139.74, 155.49. Anal. Calcd for C₃₀H₄₅NO₇S: C, 63.92; H, 8.05; N, 2.48. Found: C, 63.76; H, 7.89; N, 2.52.

cis-Adamantane-2-spiro-3′-8′-[4′-[(1′-carboxy-1′-methylethoxy)phenyl]-1′,2′,4′-trioxaspiro[4.5]decane (OZ440). To a solution of OZ452 (0.80 g, 1.70 mmol) in EtOH (50 ml) was added 2 M aq. NaOH solution (5 ml) The resulting mixture was stirred at 50° C. for 6 h. After the solvent was removed, the residue was diluted with water (10 ml) and acidified with 1 M aq. HCl (15 ml). The precipitate was collected by filtration, washed with cold water, and dried in a vacuum oven at 40° C. to afford trioxolane OZ440 (0.71 g, 95%) as a colorless solid. mp 145-146° C.; ¹H NMR (500 MHz, CDCl₃) δ 1.56 (s, 6H), 1.63-2.11 (m, 22H), 2.46-2.57 (m, 1H), 6.86 (d, J=8.3 Hz, 2H), 7.11 (d, J=8.3 Hz, 2H); ¹³C NMR (125.7 MHz, CDCl₃) δ 24.98, 26.48, 26.88, 31.51, 34.69, 34.79, 36.40, 36.79, 42.15, 79.80, 108.34, 111.39, 120.83, 127.49, 141.27, 152.33, 177.66. Anal. Calcd for C₂₆H₃₄O₆: C, 70.56; H, 7.74. Found: C, 70.38; H, 7.79.

cis-Adamantane-2-spiro-3′-8′-(4′-morpholinylmethyl)-1′,2′,4′-trioxaspiro[4.5]decane p-tosylate (OZ441). A mixture of morphline (0.17 ml, 2.0 mmol), MeOH (10 ml), HOAc (0.12 ml, 2.0 mmol), and OZ390 (146 mg, 0.50 mmol) was stirred at rt for 1 h before NaBH₃CN (133 mg, 2.0 mmol) was added. The suspension was stirred at rt for 24 h and then quenched with saturated aq. Na₂CO₃ (10 ml, pH=10). The solution was concentrated, mixed with water (10 ml), and extracted with CH₂Cl₂ (3×40 ml). The organic layers were combined, washed with water (3×40 ml), dried over MgSO₄, and evaporated to give the free base crude product (170 mg). A mixture of the free base crude product and p-toluenesulfonic acid monohydrate (88 mg, 0.45 mmol) in CH₂Cl₂ (10 ml) was stirred for 1 h and then mixed with ether (10 ml). The resulting precipitate was collected by filtration, washed with ether, and dried under vacuum to give trioxolane OZ441 (170 mg, 63%) as a white powder. mp 166-167° C.; ¹H NMR (500 MHz, DMSO-d₆) δ 1.08-1.21 (m, 2H), 1.61-1.97 (m, 21H), 2.29 (s, 3H), 2.97-3.09 (m, 4H), 3.43 (d, J=12.2 Hz, 2H), 3.71 (t, J=11.5 Hz, 2H), 3.95 (d, J=11.2 Hz, 2H), 7.12 (d, J=7.8 Hz, 2H), 7.48 (d, J=7.8 Hz, 2H), 9.21 (brs, 1H); ¹³C NMR (125.7 MHz, DMSO-d₆) δ 20.95, 25.96, 26.36, 27.58, 29.89, 32.89, 34.41, 34.44, 35.90, 36.22, 39.96, 51.78, 61.08, 63.21, 108.14, 110.87, 125.66, 128.23, 137.78, 145.90. Anal. Calcd for C₂₈H₄₁NO₇S: C, 62.78; H, 7.71; N, 2.61. Found: C, 63.02; H, 7.56; N, 2.65.

cis-Adamantane-2-spiro-3′-8′-[[(2′-hydroxyethyl)amino]methyl]-1′,2′,4′-trioxaspiro[4.5]decane p-tosylate (OZ442). A mixture of ethanolamine (0.36 ml, 6.0 mmol), MeOH (15 ml), HOAc (0.35 ml, 6.0 mmol), and OZ390 (146 mg, 0.50 mmol) was stirred at rt for 1 h before NaBH₃CN (399 mg, 6.0 mmol) was added. The suspension was stirred at rt for 16 h and then quenched with saturated aq. Na₂CO₃ (15 ml, pH=10). The solution was concentrated, diluted with water (10 ml), and extracted with CH₂Cl₂ (3×40 ml). The organic extracts were combined, washed with water (3×40 ml), dried over MgSO₄, and evaporated to give the free base crude product (190 mg). A mixture of the free base crude product and p-toluenesulfonic acid monohydrate (102 mg, 0.53 mmol) in CH₂Cl₂ (10 ml) was stirred for 1 h and then mixed with ether (10 ml). The resulting precipitate was collected by filtration, washed with ether, and dried under vacuum to give trioxolane OZ442 (160 mg, 62%) as a white powder. mp 134-136° C.; ¹H NMR (500 MHz, DMSO-d₆) δ 1.08-1.21 (m, 2H), 1.45-1.61 (m, 2H), 1.62-2.03 (m, 19H), 2.38 (s, 3H), 2.78-2.84 (m, 2H), 3.19 (brs, 2H), 3.92-4.03 (m, 2H), 7.21 (d, J=7.8 Hz, 2H), 7.72 (d, J=8.3 Hz, 2H), 8.32 (brs, 2H); ¹³C NMR (125.7 MHz, DMSO-d₆) δ 21.31, 26.45, 26.85, 27.60, 33.17, 34.75, 36.35, 36.76, 50.89, 53.52, 57.16, 107.89, 111.37, 125.79, 129.08, 140.82, 141.22.

cis-Adamantane-2-spiro-3′-8′-[4′-[3′-(dimethylamino)propoxy]phenyl]-1′,2′,4′-trioxaspiro[4.5]decane mesylate (OZ443). To a solution of OZ288 (0.50 g, 1.4 mmol) in dry acetonitrile (50 ml) were added powdered NaOH (0.225 g, 5.62 mmol) and tetrabutylammonium hydrogen sulfate (0.095 g, 0.280 mmol). The mixture was stirred at 25° C. for 30 min before 3-dimethylamino-1-propyl chloride hydrochloride (0.444 g, 2.81 mmol) was added. After the addition, it was stirred at 60° C. overnight. The inorganic solid was filtered off and washed with CH₂Cl₂. After removal of the solvents, the residue was dissolved in EtOAc (50 ml). The organic layer was washed with water and brine and dried over MgSO₄. Removal of the solvent afforded the free base. To the solution of the above free base (0.61 g, 1.38 mmol) in CH₂Cl₂ (10 ml) at 0° C. was added dropwise a solution of methanesulfonic acid (0.12 g, 1.244 mmol) in ether (10 ml). The solid obtained was filtered, washed with ether (25 ml), and dried under vacuum at 40° C. to afford trioxolane OZ443 (0.67 g, 90%) as a colorless solid. mp 148-150° C.; ¹H NMR (500 MHz, CDCl₃) δ 1.61-2.08 (m, 22H), 2.25-2.33 (m, 2H), 2.43-2.54 (m, 1H), 2.78 (s, 3H), 2.92 (d, J=4.9 Hz, 6H), 3.21-3.31 (m, 2H), 4.05 (t, J=5.7 Hz, 2H), 6.79 (d, J=8.6 Hz, 2H), 7.11 (d, J=8.3 Hz, 2H), 10.98 (brs, 1H); ¹³C NMR (125.7 MHz, CDCl₃) δ 24.60, 26.40, 26.80, 31.54, 34.62, 34.73, 36.33, 36.72, 39.13, 41.92, 43.36, 56.01, 64.39, 108.29, 111.32, 114.21, 127.69, 139.07, 156.41. Anal. Calcd for C₂₈H₄₃NO₇S: C, 62.54; H, 8.06; N, 2.60. Found: C, 62.50; H, 7.91; N, 2.65.

3-[4-(cis-Adamantane-2-spiro-3′-1′,2′,4′-trioxaspiro[4.5]decan-8′-yl)phenoxy]-N,N,N-trimethylpropanaminium mesylate (OZ444). To the solution of the free base of OZ443 (0.62 g, 1.40 mmol) in benzene (20 ml) was added dropwise a solution of methyl methanesulfonate (0.232 g, 2.1 mmol) in benzene (5 ml). The reaction mixture was stirred at rt for 48 h. The solid obtained was filtered, washed with EtOAc (25 ml), and dried under vacuum at 40° C. to afford trioxolane OZ444 (0.70 g, 91%) as a colorless solid. mp 150-152° C.; ¹H NMR (500 MHz, DMSO-d₆) δ 1.47-1.61 (m, 2H), 1.62-1.99 (m, 20H), 2.12-2.23 (m, 2H), 2.31 (s, 3H), 2.49-2.62 (m, 1H), 3.10 (s, 9H), 3.44-3.53 (m, 2H), 4.02 (t, J=5.9 Hz, 2H), 6.87 (d, J=8.8 Hz, 2H), 7.14 (d, J=8.8 Hz, 2H); ¹³C NMR (125.7 MHz, DMSO-d₆) δ 22.80, 25.98, 26.39, 31.46, 34.26, 34.42, 35.96, 36.25, 40.86, 52.38, 52.41, 52.44, 63.16, 64.90, 108.29, 110.70, 114.60, 127.67, 138.52, 156.69. Anal. Calcd for C₂₉H₄₅NO₇S: C, 63.13; H, 8.22; N, 2.54. Found: C, 63.28; H, 8.15; N, 2.56.

cis-Adamantane-2-spiro-3′-8′-[4′-[2′-(dimethylamino)ethoxy]phenyl]-1′,2′,4′-trioxaspiro[4.5]decane mesylate (OZ445). To a solution of OZ288 (1.0 g, 2.81 mmol) in dry acetonitrile (50 ml) were added powdered NaOH (0.45 g, 11.24 mmol) and tetrabutylammonium hydrogen sulfate (0.191 g, 0.562 mmol). The mixture was stirred at 25° C. for 30 min before 2-dimethylamino-1-ethyl chloride hydrochloride (0.810 g, 5.61 mmol) was added. After the addition, it was stirred at 60° C. overnight. The inorganic solid was filtered off and washed with CH₂Cl₂. After removal of the solvents, the residue was dissolved in EtOAC (50 ml). The organic layer was washed with water and brine and dried over MgSO₄. Removal of the solvent afforded the free base (1.09 g, 91%) as a colorless solid. To the solution of the above free base (0.53 g, 1.24 mmol) in CH₂Cl₂ (10 ml) at 0° C. was added dropwise a solution of methanesulfonic acid (0.107 g, 1.12 mmol) in ether (10 ml). The solid obtained was filtered, washed with ether (25 ml), and dried under vacuum at 40° C. to afford trioxolane OZ445 (0.58 g, 89%) as a colorless solid. mp 156-158° C.; ¹H NMR (500 MHz, DMSO-d₆) δ 1.49-1.61 (m, 2H), 1.63-2.01 (m, 20H), 2.33 (s, 3H), 2.52-2.64 (m, 1H), 2.86 (s, 6H), 3.50 (t, J=4.9 Hz, 2H), 4.28 (t, J=5.2 Hz, 2H), 6.93 (d, J=8.8 Hz, 2H), 7.17 (d, J=8.3 Hz, 2H), 9.59 (brs, 1H); ¹³C NMR (125.7 MHz, DMSO-d₆) δ 25.99, 26.39, 31.46, 34.26, 34.43, 35.96, 36.25, 39.91, 40.88, 43.02, 55.65, 62.34, 108.29, 110.72, 114.82, 127.73, 139.06, 156.02. Anal. Calcd for C₂₇H₄₁NO₇S: C, 61.92; H, 7.89; N, 2.67. Found: C, 61.81; H, 7.60; N, 2.68.

2-[4-(cis-Adamantane-2-spiro-3′-1′,2′,4′-trioxaspiro[4.5]decan-8′-yl)phenoxy]-N,N,N-trimethylethanaminium mesylate (OZ446). To the solution of the free base of OZ445 (0.56 g, 1.31 mmol) in benzene (20 ml) was added dropwise a solution of methyl methanesulfonate (0.217 g, 1.97 mmol) in benzene (5 ml). The reaction mixture was stirred at rt for 48 h. The solid obtained was filtered, washed with EtOAc (25 ml), and dried under vacuum at 40° C. to afford trioxolane OZ446 (0.66 g, 93%) as a colorless solid. mp 154-156° C.; ¹H NMR (500 MHz, DMSO-d₆) δ 1.48-1.61 (m, 2H), 1.62-1.97 (m, 20H), 2.31 (s, 3H), 2.52-2.63 (m, 1H), 3.18 (s, 9H), 3.77 (t, J=4.7 Hz, 2H), 4.42 (brs, 2H), 6.93 (d, J=8.8 Hz, 2H), 7.17 (d, J=8.8 Hz, 2H); ¹³C NMR (125.7 MHz, DMSO-d₆) δ 25.98, 26.39, 31.47, 34.25, 34.43, 35.96, 36.25, 40.04, 40.88, 53.26, 61.75, 64.29, 108.29, 110.72, 114.80, 127.72, 139.06, 155.87. Anal. Calcd for C₂₈H₄₃NO₇S: C, 62.54; H, 8.06; N, 2.60. Found: C, 62.44; H, 7.86; N, 2.74.

cis-Adamantane-2-spiro-3′-8′-[4′-[2′-[(2′-amino-2′-methylpropyl)amino]-1′,1′-dimethyl-2′-oxoethoxy]phenyl]-1′,2′,4′-trioxaspiro[4.5]decane p-tosylate (OZ447). Step 1. A solution of OZ440 (0.55 g, 1.26 mmol), HOSu (0.16 g, 1.39 mmol), and EDCI (0.30 g, 1.56 mmol) in DMF (10 ml) was stirred at rt for 24 h. Under ice cooling, the reaction was quenched with water (30 ml). The precipitate was collected by filtration, washed with cold water, and dried in a vacuum oven at 40° C. to afford the active ester as a colorless solid (0.61 g, 91%). mp 159-160° C. ¹H NMR (500 MHz, CDCl₃) δ 1.71 (s, 6H), 1.54-2.08 (m, 22H), 2.44-2.54 (m, 1H), 2.82-2.90 (m, 4H), 6.97 (d, J=8.5 Hz, 2H), 7.09 (d, J=8.5 Hz, 2H). Step 2. To a solution of 1,2-diamino-2-methylpropane (0.25 g, 2.8 mmol) in CHCl₃ (10 ml) was added dropwise the solution of the active ester (0.25 g, 0.47 mmol) in CHCl₃ (20 ml). The resulting mixture was stirred at rt for 4 h and then quenched with water (10 ml). After separation of the organic layer, the aqueous layer was extracted with CHCl₃ (2×20 ml). The combined extracts were washed with water (2×10 ml) and brine (10 ml), dried over MgSO₄, filtered, and concentrated. The residue was dissolved in ether (10 ml) and then the solution of p-toluenesulfonic acid monohydrate (90 mg, 0.47 mmol) in ether (10 ml) was added. The precipitate was collected by filtration to afford trioxolane OZ447 as a colorless solid (0.30 g, 94%). mp 150-152° C.; ¹H NMR (500 MHz, DMSO-d₆) δ 1.13 (s, 6H), 1.44 (s, 6H), 1.47-1.59 (m, 2H), 1.63-1.96 (m, 20H), 2.29 (s, 3H), 2.51-2.61 (m, 1H), 3.26 (d, J=6.3 Hz, 2H), 6.82 (d, J=8.3 Hz, 2H), 7.11 (d, J=7.8 Hz, 2H), 7.13 (d, J=8.3 Hz, 2H), 7.48 (d, J=7.8 Hz, 2H), 7.71 (brs, 3H), 8.28 (t, J=6.3 Hz, 1H); ¹³C NMR (125.7 MHz, DMSO-d₆) δ 20.95, 23.56, 25.26, 25.98, 26.39, 31.38, 34.25, 34.44, 35.96, 36.25, 39.96, 40.90, 46.33, 54.81, 80.32, 108.28, 110.74, 120.07, 125.67, 127.39, 128.21, 137.72, 140.03, 145.98, 152.94, 175.01. Anal. Calcd for C₃₇H₅₂N₂O₈S: C, 64.89; H, 7.65; N, 4.09. Found: C, 64.79; H, 7.88; N, 4.10.

cis-Adamantane-2-spiro-3′-8′-[4′-[1′,1′-dimethyl-2′-oxo-2′-(1′-piperazinyl)ethoxy]phenyl]-1′,2′,4′-trioxaspiro[4.5]decane p-tosylate (OZ448). To a solution of piperazine (0.20 g, 2.9 mmol) in CHCl₃ (10 ml) was added dropwise the solution of the OZ440-HOSu active ester (0.25 g, 0.47 mmol, see OZ447) in CHCl₃ (10 ml). The resulting mixture was stirred at rt for 4 h and then quenched with water (10 ml). After separation of the organic layer, the aqueous layer was extracted with CHCl₃ (2×20 ml). The combined extracts were washed with water (2×10 ml) and brine (10 ml), dried over MgSO₄, filtered, and concentrated. The residue was dissolved in ether (10 ml) and then the solution of p-toluenesulfonic acid monohydrate (80 mg, 0.42 mmol) in ether (10 ml) was added. The precipitate was collected by filtration to afford trioxolane OZ448 as a colorless solid (0.22 g, 69%). mp 135-137° C.; ¹H NMR (500 MHz, DMSO-d₆) δ 1.53 (s, 6H), 1.46-1.59 (m, 2H), 1.63-1.97 (m, 20H), 2.29 (s, 3H), 2.51-2.62 (m, 1H), 2.74 (brs, 2H), 3.02 (brs, 2H), 3.70 (brs, 2H), 3.94 (brs, 2H), 6.73 (d, J=8.8 Hz, 2H), 7.12 (d, J=7.8 Hz, 2H), 7.14 (d, J=8.3 Hz, 2H), 7.48 (d, J=8.3 Hz, 2H), 8.62 (brs, 2H); ¹³C NMR (125.7 MHz, DMSO-d₆) δ 20.96, 25.89, 25.99, 26.40, 31.35, 34.25, 34.44, 35.96, 36.25, 39.79, 39.96, 40.29, 40.83, 42.99 (br), 80.52, 108.28, 110.74, 117.36, 125.67, 127.82, 128.21, 137.79, 139.56, 145.89, 153.14, 171.04.

cis-Adamantane-2-spiro-3′-8′-[4′-[3′-[(aminoacetyl)amino]propoxy]phenyl]-1′,2′,4′-trioxaspiro[4.5]decane mesylate (OZ449). Step 1. To a solution of N-Boc-Glycine (0.565 g, 3.23 mmol), HOBT (0.545 g, 4.03 mmol), and triethylamine (0.57 ml, 4.03 mmol) in dry DMF (30 ml) at 0° C. was added EDCI (0.773 g, 4.03 mmol). After the reaction mixture was stirred at 0° C. for 10 min, the free base of OZ401 (1.11 g, 2.68 mmol) was added. The reaction mixture was allowed to warm up to rt and stirred at rt overnight before it was poured on to chopped ice. The solid obtained was filtered, washed with water, and dried under vacuum at 40° C. to afford the N-Boc-protected amide (1.4 g, 96%) as a colorless solid. Step 2. The above N-Boc-protected amide (0.96 g, 1.76 mmol) was added to the solution of 1.5 M methanesulfonic acid in THF (12 ml). The reaction mixture was stirred overnight. The solid obtained was filtered, washed with ether, and dried under vacuum at 40° C. to afford trioxolane OZ449 (1.0 g, 100%) as a colorless solid. mp 152-154° C.; ¹H NMR (500 MHz, DMSO-d₆) δ 1.46-1.59 (m, 2H), 1.61-1.97 (m, 22H), 2.36 (s, 3H), 2.47-2.62 (m, 1H), 3.24-3.33 (m, 2H), 3.52-3.57 (m, 2H), 3.96 (t, J=6.1 Hz, 2H), 6.84 (d, J=8.3 Hz, 2H), 7.11 (d, J=8.3 Hz, 2H), 7.98 (brs, 3H), 8.42 (t, J=5.2 Hz, 1H); ¹³C NMR (125.7 MHz, DMSO-d₆) δ 26.01, 26.42, 28.88, 31.51, 34.30, 34.45, 35.86, 35.98, 36.28, 39.89, 40.36, 40.89, 65.06, 108.33, 110.72, 114.55, 127.60, 138.14, 157.00, 166.04. Anal. Calcd for C₂₈H₄₂N₂O₈S: C, 59.34; H, 7.47; N, 4.94. Found: C, 58.91; H, 7.73; N, 5.10.

cis-Adamantane-2-spiro-3′-8′-[4′-(2′-hydroxy-1′,1′-dimethylethoxy)phenyl]-1′,2′,4′-trioxaspiro[4.5]decane (OZ450). To a solution of OZ452 (1.5 g, 3.19 mmol) in ether (10 ml) and THF (4 ml) was added dropwise 2 M lithium borohydride in THF (3.9 ml, 7.8 mmol) followed by 1 M lithium triethylborohydride in THF (0.78 ml, 0.78 mmol). The resulting mixture was stirred at rt for 24 h and then diluted with ether (30 ml). The mixture was washed with 2 M aq. NaOH (2×5 ml), water (2×5 ml) and brine (5 ml), dried over MgSO₄, filtered, and concentrated to afford trioxolane alcohol OZ450 (1.3 g, 95%) as a colorless solid. mp 147-148° C.; ¹H NMR (500 MHz, CDCl₃) δ 1.26 (s, 6H), 1.65-2.09 (m, 22H), 2.16 (t, J=6.4 Hz, 1H), 2.46-2.56 (m, 1H), 3.58 (d, J=6.4 Hz, 2H), 6.90 (d, J=8.3 Hz, 2H), 7.10 (d, J=8.3 Hz, 2H); ¹³C NMR (125.7 MHz, CDCl₃) δ 23.11, 26.50, 26.89, 31.55, 34.74, 34.81, 36.42, 36.81, 42.25, 70.30, 80.37, 108.39, 111.39, 123.70, 127.28, 141.57, 152.67. Anal. Calcd for C₂₆H₃₆O₅: C, 72.87; H, 8.47. Found: C, 72.66; H, 8.56.

cis-Adamantane-2-spiro-3′-8′-[[(2′-hydroxy-2′-methylpropyl)amino]methyl]-1′,2′,4′-trioxaspiro[4.5]decane p-tosylate (OZ451). A solution of 1,2-epoxy-2-methylpropane (72 mg, 1.0 mmol) and the free base of OZ209 (293 mg, 1.0 mmol) in EtOH (4 ml) was stirred at rt for 40 h. After EtOH was removed, the residue was dissolved in CH₂Cl₂ (40 ml), washed with water (3×20 ml), dried over MgSO₄, and evaporated to give the free base crude product (345 mg, 94%). A mixture of the free base crude product and p-toluenesulfonic acid monohydrate (190 mg, 1.0 mmol) in CH₂Cl₂ (20 ml) was stirred for 1 h and then evaporated to dryness. The residue was washed with ether (5×10 ml) to give trioxolane OZ451 (195 mg, 36%) as a white powder. mp 157-158° C.; ¹H NMR (500 MHz, DMSO-d₆) δ 1.04-1.19 (m, 2H), 1.20 (s, 6H), 1.61-1.97 (m, 21H), 2.29 (s, 3H), 2.76-2.91 (m, 4H), 5.13 (s, 1H), 7.12 (d, J=7.8 Hz, 2H), 7.48 (d, J=7.8 Hz, 2H), 8.06 (brs, 2H); ¹³C NMR (125.7 MHz, DMSO-d₆) δ 20.94, 25.96, 26.36, 27.46, 27.55, 31.93, 33.08, 34.41, 34.43, 35.90, 36.23, 53.01, 57.20, 67.36, 108.25, 110.81, 125.66, 128.20, 137.74, 145.94. Anal. Calcd for C₂₈H₄₃NO₇S: C, 62.54; H, 8.06; N, 2.60. Found: C, 62.54; H, 7.90; N, 2.51.

cis-Adamantane-2-spiro-3′-8′-[4′-[2′-ethoxy-1′,1′-dimethyl-2′-oxoethoxy]phenyl]-1′,2′,4′-trioxaspiro[4.5]decane (OZ452). A mixture of OZ288 (3.6 g, 10 mmol), ethyl 2-bromoisobutyrate (2.2 ml, 15 mmol), and anhydrous potassium carbonate (2.76 g, 20 mmol) in acetone (200 ml) was refluxed for 5 d. The reaction mixture was cooled to rt, filtered to remove the solid, and concentrated. The crude product was purified by chromatography (silica gel, 10% ether in hexanes) to afford trioxolane OZ452 (2.1 g, 45%) as a colorless solid. mp 77-79° C.; ¹H NMR (500 MHz, CDCl₃) δ 1.24 (t, J=7.2 Hz, 3H), 1.57 (s, 6H), 1.61-2.09 (m, 22H), 2.45-2.54 (m, 1H), 4.23 (q, J=7.3 Hz, 2H), 6.76 (d, J=8.8 Hz, 2H), 7.05 (d, J=8.8 Hz, 2H); ¹³C NMR (125.7 MHz, CDCl₃) δ 14.05, 25.37, 26.49, 26.89, 31.54, 34.73, 34.79, 34.80, 36.41, 36.81, 42.08, 61.32, 79.03, 108.41, 111.34, 119.17, 127.27, 139.85, 153.61, 174.38. Anal. Calcd for C₂₈H₃₈O₆: C, 71.46; H, 8.14. Found: C, 71.65; H, 8.06.

cis-Adamantane-2-spiro-3′-8′-(3′-hydroxyphenyl)-1′,2′,4′--trioxaspiro[4.5]decane (OZ454). Step 1. A mixture of m-bromophenol (9.1 g, 52.6 mmol), benzyl bromide (9.4 ml, 78.9 mmol), and anhydrous potassium carbonate (15 g, 109 mmol) in acetone (80 ml) was refluxed overnight. The reaction mixture was cooled to rt, filtered to remove the solid, and concentrated. The crude product was purified by chromatography (silica gel, 2% ether in hexanes) to afford 1-benzyloxy-3-bromobenzene (11.7 g, 85%) as a colorless solid. ¹H NMR (500 MHz, CDCl₃) δ 5.03 (s, 2H), 6.87-6.93 (m, 1H), 7.06-7.18 (m, 3H), 7.26-7.46 (m, 5H). Step 2. To a 250 ml round-bottom flask equipped with a stirrer, condenser, and addition funnel was added magnesium turnings (1.22 g, 50 mmol) followed by enough THF to cover the Mg. A solution of 1-benzyloxy-3-bromobenzene (6.6 g, 25 mmol) in THF (70 ml) was added dropwise at such a rate that the reaction maintained a gentle reflux. After the mixture was refluxed for an additional 2 h, a solution of 1,4-cyclohexanedione monoethylene ketal (3.5 g, 22 mol) in THF (70 ml) was added dropwise. The mixture was refluxed overnight before being quenched with saturated NH₄Cl (1 ml). After removal of magnesium salts by filtration, the filtrate was concentrated to dryness. The residue was dissolved in CH₂Cl₂ and washed with water and brine. The organic layer was separated, dried over MgSO₄, filtered, and concentrated. The crude product was purified by chromatography (silica gel, 30% ether in hexanes) to afford the desired alcohol (5.2 g, 68%) as a colorless oil. ¹H NMR (500 MHz, CDCl₃) δ 1.66-1.74 (m, 2H), 1.78-1.84 (m, 2H), 2.05-2.23 (m, 4H), 3.94-4.04 (m, 4H), 5.07 (s, 2H), 6.85-6.90 (m, 1H), 7.08-7.17 (m, 1H), 7.18-7.22 (m, 1H), 7.24-7.30 (m, 1H), 7.30-7.48 (m, 5H). Step 3. To a solution of the above alcohol (5.2 g, 15.3 mmol) and triethylamine (11 ml, 79 mmol) in CH₂Cl₂ (220 ml) at 0° C. was added dropwise methanesulfonyl chloride (2.43 ml, 31.4 mmol). After being stirred at 0° C. for 5 h and at rt overnight, the reaction mixture was washed with water and brine, dried over MgSO₄, filtered, and concentrated. The crude product was purified by chromatography (silica gel, 30% ether in hexanes) to afford the desired olefin (2.05 g, 67%) as a pale yellow oil. ¹H NMR (500 MHz, CDCl₃) δ 1.91 (t, J=6.8 Hz, 2H), 2.44-2.49 (m, 2H), 2.61-2.68 (m, 2H), 4.02 (s, 4H), 5.06 (s, 2H), 5.96-6.02 (m, 1H), 6.83-6.87 (m, 1H), 6.98-7.05 (m, 2H), 7.21 (t, J=7.8 Hz, 1H), 7.30-7.48 (m, 5H); ¹³C NMR (125.7 MHz, CDCl₃) δ 26.81, 31.33, 36.15, 64.47, 69.97, 107.73, 112.13, 113.11, 118.08, 121.82, 127.49, 127.90, 128.56, 129.10, 136.16, 137.10, 143.08, 158.73. Step 4. To a solution of the above olefin (1.1 g, 3.4 mmol) in EtOAc (60 ml) was added 10% palladium/carbon (0.2 g). The resulting mixture was hydrogenated with a H₂-balloon at rt for 24 h. The mixture was filtered through Celite and concentrated to afford the phenol product (0.80 g, 100%) as a colorless oil. ¹H NMR (500 MHz, CDCl₃) δ 1.60-1.96 (m, 8H), 2.48-2.60 (m, 1H), 3.98 (s, 4H), 6.66 (dd, J=2.8, 8.5 Hz, 1H), 6.72 (d, J=1.5 Hz, 1H), 6.81 (d, J=7.5 Hz, 1H), 7.15 (t, J=7.8 Hz, 1H). Step 5. To a solution of dry pyridine (20 ml) and acetic anhydride (8 ml) at −70° C. was added a solution of the above phenol (0.80 g, 3.4 mmol) in EtOAc (10 ml). The reaction mixture was stored at −30° C. overnight. After removal of the solvents, the residue was partitioned between CH₂Cl₂ (30 ml) and water (30 ml). The aqueous layer was extracted with CH₂Cl₂ (2×30 ml). The combined extracts were washed with 1 M aq. HCl (2×10 ml) and water (10 ml), dried over MgSO₄, filtered, and concentrated to afford the acetylated product (0.90 g, 96%) as a colorless oil. ¹H NMR (500 MHz, CDCl₃) δ 1.60-1.96 (m, 8H), 2.29 (s, 3H), 2.52-2.62 (m, 1H), 3.98 (s, 4H), 6.91 (d, J=7.5 Hz, 1H), 6.96 (s, 1H), 7.10 (d, J=7.5 Hz, 1H), 7.26-7.30 (m, 1H). Step 6. A mixture of the above ketal (1.2 g, 4.35 mmol) and PPTS (0.10 g) in acetone (40 ml) and water (8 ml) was refluxed for 2 d. After removal of acetone, the residue was partitioned between CH₂Cl₂ (30 ml) and water (30 ml). The aqueous layer was extracted with CH₂Cl₂ (2×30 ml). The combined extracts were washed with water and brine, dried over MgSO₄, filtered, and concentrated to afford 4-(3-acetoxyphenyl)cyclohexanone (0.90 g, 89%) as a colorless oil. ¹H NMR (500 MHz, CDCl₃) δ 1.82-2.00 (m, 2H), 2.20-2.30 (m, 2H), 2.30 (s, 3H), 2.40-2.60 (m, 4H), 3.00-3.10 (m, 1H), 6.90-7.00 (m, 2H), 7.10-7.20 (m, 1H), 7.30-7.40 (m, 1H); ¹³C NMR (125.7 MHz, CDCl₃) δ 21.15, 33.77, 41.22, 42.48, 119.75, 119.89, 124.19, 129.54, 146.48, 150.87, 169.48, 210.77. Step 7. A solution of O-methyl 2-adamantanone oxime (1.04 g, 5.81 mmol) and 4-(3-acetoxyphenyl)cyclohexanone (0.90 g, 3.88 mmol) in cyclohexane (120 ml) and CH₂Cl₂ (40 ml) was treated with ozone according to the general procedure. After removal of the solvents, the crude product was purified by crystallization from EtOH to afford adamantane-2-spiro-3′-8′-(3′-acetoxyphenyl)-1′,2′,4′-trioxaspiro[4.5]decane (0.81 g, 53%) as a colorless solid. mp 110-111° C. ¹H NMR (500 MHz, CDCl₃) δ 1.66-2.10 (m, 22H), 2.29 (s, 3H), 2.50-2.60 (m, 1H), 6.89-6.95 (m, 2H), 7.04-7.10 (m, 1H), 7.24-7.32 (m, 1H); ¹³C NMR (125.7 MHz, CDCl₃) δ 21.13, 26.48, 26.88, 31.27, 34.62, 34.79, 34.80, 36.40, 36.80, 42.68, 108.24, 111.42, 119.28, 119.99, 124.22, 129.28, 147.90, 150.73, 169.49. Step 8. To a solution of adamantane-2-spiro-3′-8′-(3′-acetoxyphenyl)-1′,2′,4′-trioxaspiro[4.5]decane (0.80 g, 1.2 mmol) in MeOH (8 ml) and THF (4 ml) was added 15% aq. KOH solution (1.3 ml) The resulting mixture was stirred at 50° C. for 4 h. After the solution was concentrated to 5 ml, the residue was diluted with water (10 ml) and acidified with acetic acid (1 ml). The precipitate was collected by filtration, washed with cold water, and dried in a vacuum oven at 40° C. to afford trioxolane OZ454 (0.61 g, 85%) as a colorless solid. mp 120-121° C.; ¹H NMR (500 MHz, CDCl₃) δ 1.63-2.09 (m, 22H), 2.45-2.54 (m, 1H), 4.66 (brs, 1H), 6.63-6.69 (m, 2H), 6.78 (d, J=7.3 Hz, 1H), 7.15 (t, J=7.8 Hz, 1H); ¹³C NMR (125.7 MHz, CDCl₃) δ 26.49, 26.89, 31.31, 34.68, 34.81, 36.41, 36.81, 42.80, 108.36, 111.42, 113.02, 113.70, 119.36, 129.56, 148.22, 155.50. Anal. Calcd for C₂₂H₂₈O₄: C, 74.13; H, 7.92. Found: C, 74.33; H, 7.90.

cis-Adamantane-2-spiro-3′-8′-[3′-(3′-aminopropoxy)phenyl]-1′,2′,4′-trioxaspiro[4.5]decane p-tosylate (OZ455). To a solution of OZ454 (0.25 g, 0.70 mmol) in dry acetonitrile (15 ml) were added powered NaOH (0.17 g, 4.25 mmol) and tetrabutylammonium hydrogen sulfate (0.05 g, 0.14 mmol). The mixture was stirred at rt for 30 min before 3-chloropropylamine hydrochloride (0.28 g, 2.1 mmol) was added. The reaction mixture was stirred at 60° C. overnight, cooled to rt, filtered, and washed with CH₂Cl₂. After the filtrate was concentrated, the residue was dissolved in CH₂Cl₂, washed with water and brine, and dried over MgSO₄, filtered, and concentrated. The residue was dissolved in CH₂Cl₂ (5 ml) and then the solution of p-toluenesulfonic acid monohydrate (0.13 g) in ether (20 ml) was added. The precipitate was collected by filtration to afford trioxolane OZ455 (0.34 g, 83%) as a colorless solid. mp 147-148° C.; ¹H NMR (500 MHz, DMSO-d₆) δ 1.55-2.07 (m, 24H), 2.29 (s, 3H), 2.45-2.54 (m, 1H), 2.96 (t, J=7.3 Hz, 2H), 4.03 (t, J=6.2 Hz, 2H), 6.72-6.78 (m, 2H), 6.80 (d, J=7.3 Hz, 1H), 7.11 (d, J=7.8 Hz, 2H), 7.20 (t, J=7.8 Hz, 1H), 7.48 (d, J=8.3 Hz, 2H), 7.69 (brs, 3H); ¹³C NMR (125.7 MHz, DMSO-d₆) δ 20.95, 25.99, 26.40, 27.08, 31.20, 34.23, 34.44, 35.96, 36.25, 36.65, 41.79, 64.53, 108.28, 110.76, 112.28, 112.88, 119.27, 125.66, 128.21, 129.59, 137.77, 145.91, 147.80, 158.52. Anal. Calcd for C₃₂H₄₃NO₇S: C, 65.62; H, 7.40; N, 2.39. Found: C, 65.52; H, 7.60; N, 2.42.

Adamantane-2-spiro-3′-5′-(4′-cyanophenyl)-5′-methyl-1′,2′,4′-trioxolane (OZ456). A solution of O-methyl 2-adamantanone oxime (2.7 g, 15 mmol) and 4-acetylbenzonitrile (2.2 g, 15.2 mmol) in cyclohexane (90 ml) and CH₂Cl₂ (30 ml) was treated with ozone according to the general procedure. After removal of the solvents, the crude product was purified by chromatography (silica gel, 2% ether in hexanes) to afford trioxolane OZ456 (2.55 g, 54%) as a colorless solid. mp 90-91° C.; ¹H NMR (500 MHz, CDCl₃) δ 1.69 (s, 3H), 1.57-2.22 (m, 14H), 7.59-7.69 (m, 4H); ¹³C NMR (125.7 MHz, CDCl₃) δ 25.21, 26.26, 26.71, 34.13, 34.58, 34.64, 35.30, 35.44, 36.42, 36.57, 107.71, 111.94, 113.47, 118.58, 125.96, 132.00, 148.24. Anal. Calcd for C₁₉H₂₁NO₃: C, 73.29; H, 6.80; N, 4.50. Found: C, 73.26; H, 6.86; N, 4.45.

cis-Adamantane-2-spiro-3′-8′-[4′-(4′-aminobutoxy)phenyl]-1′,2′,4′-trioxaspiro[4.5]decane mesylate (OZ457). Step 1. To a solution of OZ288 (0.50 g, 1.40 mmol) in dry acetonitrile (30 ml) were added powdered K₂CO₃ (0.97 g, 7.02 mmol) and tetrabutylammonium hydrogen sulfate (0.095 g, 0.28 mmol). The mixture was stirred at 25° C. for 30 min before N-(4-bromobutyl)-phthalimide (0.475 g, 1.69 mmol) was added. After the addition, it was stirred at 60° C. overnight. The inorganic solid was filtered off and washed with CH₂Cl₂. After removal of the solvents, the residue was dissolved in EtOAc (50 ml). The organic layer was washed with water and brine and dried over MgSO₄. Removal of the solvent afforded the phthalimido derivative (0.70 g, 89%) as a colorless solid (0.70 g, 89%). Step 2. To a solution of the above phthalimide (0.70 g, 1.26 mmol) in chloroform-ethanol (7:3, 30 ml) at rt was added dropwise hydrazine hydrate (0.63 g, 12.6 mmol). Then, it was stirred at 60° C. overnight. The solid was filtered off and washed with chloroform (20 ml). After the combined organic layers were concentrated, the residue obtained was dissolved in EtOAc (50 ml), washed with water, and dried over MgSO₄. Removal of the solvent afforded the free base. To the solution of the above free base in CH₂Cl₂ (10 ml) was added dropwise the solution of methanesulfonic acid (0.121 g, 1.26 mmol) in ether (10 ml). The solid obtained was filtered, washed with ether, and dried under vacuum at 40° C. to afford trioxolane OZ457 (0.60 g, 92%) as a colorless solid. mp 146-148° C.; ¹H NMR (500 MHz, DMSO-d₆) δ 1.46-1.61 (m, 2H), 1.62-2.01 (m, 24H), 2.36 (s, 3H), 2.49-2.62 (m, 1H), 2.86 (q, J=6.3 Hz, 2H), 3.94 (t, J=5.6 Hz, 2H), 6.84 (d, J=8.3 Hz, 2H), 7.11 (d, J=8.8 Hz, 2H), 7.73 (brs, 3H); ¹³C NMR (125.7 MHz, DMSO-d₆) δ 24.14, 25.86, 26.00, 26.41, 31.50, 34.29, 34.44, 35.97, 36.27, 38.85, 39.92, 40.88, 66.94, 108.31, 110.71, 114.49, 127.61, 138.12, 156.99. Anal. Calcd for C₂₇H₄₁NO₇S: C, 61.92; H, 7.89; N, 2.67. Found: C, 61.84; H, 8.00; N, 2.75.

cis-Adamantane-2-spiro-3′-8′-[4′-[3′-(1′-piperazinyl)propoxy]phenyl]-1′,2′,4′-trioxaspiro[4.5]decane dimesylate (OZ458). To a solution of OZ288 (0.50 g, 1.40 mmol) in dry acetonitrile (30 ml) were added powdered NaOH (0.225 g, 5.62 mmol) and tetrabutylammonium hydrogen sulfate (0.095 g, 0.28 mmol). The mixture was stirred at 25° C. for 30 min before 1-(3-chloropropyl)piperazine dihydrochloride (0.515 g, 2.1 mmol) was added. After the addition, it was stirred at 60° C. overnight. The inorganic solid was filtered off and washed with CH₂Cl₂. After removal of the solvents, the residue was dissolved in EtOAc (50 ml). The organic layer was washed with water and brine and dried over MgSO₄. Removal of the solvent afforded the free base. To the solution of the above free base in CH₂Cl₂ (10 ml) at 0° C. was added dropwise a solution of methanesulfonic acid (0.045 g, 0.46 mmol) in ether (10 ml). The solid obtained was filtered, washed with ether (25 ml), and dried under vacuum at 40° C. to afford trioxolane OZ458 (0.265 g, 100%) as a colorless solid. mp 125-130° C.; ¹H NMR (500 MHz, DMSO-d₆) δ 1.46-1.61 (m, 2H), 1.62-1.98 (m, 20H), 2.08-2.19 (m, 2H), 2.43 (s, 6H), 2.49-2.62 (m, 1H), 3.19-3.82 (m, 10H), 4.03 (t, J=5.9 Hz, 2H), 6.87 (d, J=8.8 Hz, 2H), 7.13 (d, J=8.8 Hz, 2H), 9.12 (brs, 2H), 10.11 (brs, 1H); ¹³C NMR (125.7 MHz, DMSO-d₆) δ 23.62, 26.00, 26.41, 31.48, 34.28, 34.44, 35.97, 36.27, 39.91, 40.46, 40.87, 48.32, 53.61, 64.77, 108.31, 110.72, 114.62, 127.66, 138.48, 156.67.

cis-Adamantane-2-spiro-3′-8′-[4′-[3′-[(2′-amino-2′-methylpropyl)amino]propoxy]phenyl]-1′,2′,4′-trioxaspiro[4.5]decane dimesylate (OZ459). Step 1. To a solution of OZ288 (1.0 g, 2.8 mmol) in dry acetonitrile (50 ml) were added powdered NaOH (0.225 g, 5.61 mmol) and tetrabutylammonium hydrogen sulfate (0.19 g, 0.56 mmol). The mixture was stirred at 25° C. for 30 min before 1,3-dibromopropane (1.14 g, 5.61 mmol) was added. After the addition, it was stirred at 60° C. overnight. The inorganic solid was filtered off and washed with CH₂Cl₂. After removal of the solvents, the residue was dissolved in EtOAc (50 ml). The organic layer was washed with water and brine, and dried over MgSO₄. After removal of the solvent, the residue was purified by chromatography (silica gel, 10% EtOAc in hexanes) to give the inseparable 1:1-mixture of the desired alkylated bromide and olefin product (0.364 g). Step 2. To a solution of the above mixture (0.364 g, 0.76 mmol) and triethylamine (0.53 ml, 3.81 mmol) in acetonitrile (15 ml) was added dropwise the solution of 1,2-diamino-2-methylpropane (0.336 g, 3.81 mmol) in acetonitrile (5 ml). After the reaction mixture was stirred overnight, it was quenched with water (5 ml). The solvent was removed and the residue was dissolved in EtOAc (25 ml). The organic layer was washed with water, dried over MgSO₄, and concentrated. After the residue was then dissolved in CH₂Cl₂ (10 ml) at 0° C., a solution of methanesulfonic acid (0.073 g, 0.76 mmol) in ether (10 ml) was added dropwise. The solid obtained was filtered, washed with ether, and dried under vacuum at 40° C. to afford trioxolane OZ459 (0.180 g, 41%) as a colorless solid. mp 115-120° C.; ¹H NMR (500 MHz, DMSO-d₆) δ 1.37 (s, 6H), 1.45-1.61 (m, 2H), 1.62-1.98 (m, 20H), 2.03-2.17 (m, 2H), 2.38 (s, 6H), 2.47-2.61 (m, 1H), 3.07-3.22 (m, 4H), 4.05 (t, J=5.4 Hz, 2H), 6.87 (d, J=8.8 Hz, 2H), 7.14 (d, J=8.3 Hz, 2H), 8.14 (brs, 3H), 8.51 (brs, 2H); ¹³C NMR (125.7 MHz, DMSO-d₆) δ 21.22, 23.69, 25.99, 26.40, 31.50, 34.27, 34.44, 35.97, 36.26, 39.92, 40.87, 46.58, 52.21, 54.21, 64.77, 108.31, 110.73, 114.57, 127.67, 138.44, 156.68.

cis-Adamantane-2-spiro-3′-8′-[4′-[2′-(1′,1′-dioxido-4′-thiomorpholinyl)ethoxy]phenyl]-1′,2′,4′-trioxaspiro[4.5]decane mesylate (OZ463). Step 1. To a solution of ethanolamine (1.12 g, 18.3 mmol) in water (20 ml) was added divinylsulfone (2.4 g, 20.3 mmol). After the reaction mixture was refluxed for 0.5 h, the solvent was removed in vacuo and the residue was dried at 50° C. To a solution of the above residue in 1,2-dichloroethane (50 ml) was added thionyl chloride (10 ml). After the reaction mixture was refluxed for 3 h, the resulting precipitate was filtered and washed with ether (3×25 ml), and dried at 50° C. to afford 4-(2-chloroethyl)thiomorpholine 1,1-dioxide hydrochloride (3.90 g, 91%) as a colorless solid. ¹H NMR (500 MHz, DMSO-d₆) δ 3.57 (t, J=6.4 Hz, 2H), 3.62-3.78 (m, 8H), 4.03 (t, J=6.8 Hz, 2H), 11.00 (brs, 1H). Step 2. To a solution of OZ288 (0.50 g, 1.4 mmol) in dry acetonitrile (50 ml) were added powdered NaOH (0.225 g, 5.61 mmol) and tetrabutylammonium hydrogensulfate (0.095 g, 0.28 mmol). After the reaction mixture was stirred at rt for 30 min, 4-(2-chloroethyl)thiomorpholine 1,1-dioxide hydrochloride (0.33 g, 1.4 mmol) was added. The mixture was stirred at 60° C. overnight before the inorganic solid was filtered off and washed with EtOAc (2×25 ml). After removal of the solvents in vacuo, the residue was dissolved in EtOAc (50 ml). The organic layer was washed with water, brine, dried over MgSO₄, and concentrated. The residue was purified by chromatography (silica gel, 50% EtOH in EtOAc) to afford OZ463 free base (0.25 g, 69%) as a colorless solid. To a solution of OZ463 free base (0.25 g, 0.48 mmol) in EtOAc (10 ml) at 0° C. was added dropwise a solution of methanesulfonic acid (0.05 g, 0.48 mmol) in ether (10 ml). The resulting precipitate was filtered, washed with ether (25 ml), and dried in vacuo at 40° C. to afford trioxolane OZ463 (0.25 g, 84%) as a colorless solid. mp 136-140° C.; ¹H NMR (500 MHz, DMSO-d₆) δ 1.45-1.98 (m, 22H), 2.40 (s, 3H), 2.52-2.62 (m, 1H), 3.59 (brs, 4H), 3.67 (brs, 2H), 3.79 (brs, 4H), 4.32 (t, J=4.7 Hz, 2H), 6.94 (J=8.8 Hz, 2H), 7.17 (J=8.3 Hz, 2H); ¹³C NMR (125.7 MHz, DMSO-d₆) δ 26.00, 26.41, 31.47, 34.26, 34.44, 35.97, 36.27, 40.90, 48.00, 50.99, 54.40, 59.93, 62.69, 108.30, 110.73, 114.89, 127.72, 139.11, 156.01. Anal. Calcd for C₂₉H₄₃NO₉S₂: C, 56.75; H, 7.06; N, 2.28. Found: C, 56.82; H, 7.04; N, 2.25.

cis-Adamantane-2-spiro-3′-8′-[4′-[3′-(1′,1′-dioxido-4′-morpholinyl)propoxy]phenyl]-1′,2′,4′-trioxaspiro[4.5]decane mesylate (OZ465). Step 1. To a solution of 3-amino-1-propanol (1.08 g, 14.4 mmol) in water (25 ml) was added divinylsulfone (2.0 g, 16.9 mmol). After the reaction mixture was refluxed for 0.5 h, it was concentrated in vacuo and dried at 50° C. To a solution of the above residue in 1,2-dichloroethane (50 ml) was added thionyl chloride (10 ml). After the reaction mixture was refluxed for 3 h, the resulting precipitate was filtered and washed with ether (3×25 ml) and dried at 50° C. to afford 4-(3-chloropropyl)thiomorpholine 1,1-dioxide hydrochloride (3.60 g, 100%) as a colorless solid. ¹H NMR (500 MHz, DMSO-d₆) δ 2.18-2.28 (m, 2H), 3.24-3.34 (m, 2H), 3.41-4.01 (m, 10H), 12.20 (brs, 1H). Step 2. To a solution of OZ288 (0.50 g, 1.4 mmol) in dry acetonitrile (50 ml) were added powdered NaOH (0.225 g, 5.61 mmol) and tetrabutylammonium hydrogensulfate (0.095 g, 0.28 mmol). After the reaction mixture was stirred at rt for 30 min, 4-(3-chloropropyl)thiomorpholine 1,1-dioxide hydrochloride (0.52 g, 2.1 mmol) was added. After stirring at 60° C. overnight, the inorganic solid was filtered off and washed with EtOAc (2×25 ml). After removal of the solvents in vacuo, the residue was dissolved in EtOAc (50 ml). The organic layer was washed with water, brine and dried over MgSO₄. After removal of the solvent in vacuo, the residue was purified by crystallization from EtOAc to afford OZ465 free base (0.525 g, 70%) as a colorless solid. To a solution of OZ465 free base (0.525 g, 1.0 mmol) in EtOAc (10 ml) at 0° C. was added dropwise a solution of methanesulfonic acid (0.14 g, 1.4 mmol) in ether (10 ml). The resulting precipitate was filtered, washed with ether (25 ml), and dried in vacuo at 40° C. to afford trioxolane OZ465 (0.55 g, 89%) as a colorless solid. mp 160-164° C.; ¹H NMR (500 MHz, DMSO-d₆) δ 1.45-1.98 (m, 22H), 2.08-2.18 (m, 2H), 2.40 (s, 3H), 2.51-2.61 (m, 1H), 3.31-3.85 (m, 10H), 4.02 (t, J=5.8 Hz, 2H), 6.87 (d, J=8.3 Hz, 2H), 7.13 (d, J=8.3 Hz, 2H), 10.10 (brs, 1H); ¹³C NMR (125.7 MHz, DMSO-d₆) δ 24.29, 25.99, 26.40, 31.48, 34.27, 34.44, 35.97, 36.27, 40.87, 47.97, 50.56, 53.01, 64.88, 108.31, 110.72, 114.61, 127.65, 138.47, 156.67. Anal. Calcd for C₃₀H₄₅NO₉S₂: C, 57.39; H, 7.22; N, 2.23. Found: C, 57.60; H, 7.34; N, 2.26.

Adamantane-2-spiro-3′-5′-[2′-(ethoxycarbonyl)ethyl]-5′-methyl-1′,2′,4′-trioxolane (OZ467). A solution of O-methyl 2-adamantanone oxime (5.0 g, 28 mmol) and ethyl levulinate (8.0 g, 55.6 mmol) in cyclohexane (200 ml) and CH₂Cl₂ (50 ml) was treated with ozone according to the general procedure. After removal of the solvents, the crude product was purified by chromatography (silica gel, hexane) to afford trioxolane OZ467 (5.6 g, 65%) as a colorless oil. ¹H NMR (500 MHz, CDCl₃) δ 1.26 (t, J=7.1 Hz, 3H), 1.45 (s, 3H), 1.65-2.01 (m, 14H), 2.02-2.17 (m, 2H), 2.44 (t, J=8.1 Hz, 2H), 4.08-4.19 (m, 2H); ¹³C NMR (125.7 MHz, CDCl₃) δ 14.20, 23.42, 26.41, 26.80, 29.16, 32.52, 34.71, 34.73, 34.78, 34.85, 36.00, 36.39, 36.74, 60.49, 108.94, 111.99, 173.07. Anal. Calcd for C₁₇H₂₆O₅: C, 65.78; H, 8.44. Found: C, 65.65; H, 8.21.

Adamantane-2-spiro-3′-5′-(2′-carboxyethyl)-5′-methyl-1′,2′,4′-trioxolane (OZ468). To a solution of OZ467 (0.50 g, 1.60 mmol) in EtOH (30 ml) was added 1 M aq. NaOH solution (5 ml) The resulting mixture was stirred at 50° C. for 6 h. After the solvent was removed, the residue was diluted with water (10 ml) and acidified with 1 M aq. HCl (15 ml). The precipitate was collected by filtration, washed with cold water, and dried in a vacuum oven at 40° C. to afford trioxolane OZ468 (0.41 g, 91%) as a colorless solid. mp 72-73° C.; ¹H NMR (500 MHz, CDCl₃) δ 1.46 (s, 3H), 1.65-2.02 (m, 14H), 2.05-2.18 (m, 2H), 2.48-2.54 (m, 2H); ¹³C NMR (125.7 MHz, CDCl₃) δ 23.39, 26.39, 26.78, 28.70, 32.25, 34.69, 34.72, 34.78, 34.82, 36.01, 36.38, 36.71, 108.75, 112.13, 178.17. Anal. Calcd for C₁₅H₂₂O₅: C, 63.81; H, 7.85. Found: C, 64.02; H, 7.74.

cis-Adamantane-2-spiro-3′-8′-[[[[3′-[(7′-chloro-4′-quinolinyl)amino]propyl]amino]carbonyl]methyl]-1′,2′,4′-trioxaspiro[4.5]decane (OZ469). To a solution of N-(7-chloro-4-quinolinyl)-1,3-propanediamine (236 mg, 1.0 mmol) and triethylamine (202 mg, 2.0 mmol) in CH₂Cl₂ (10 ml) was added dropwise a solution of OZ78-HOBt active ester (439 mg, 1.0 mmol) in CH₂Cl₂ (10 ml) The mixture was stirred at rt for 1.5 h before it was quenched with water (20 ml). After the organic layer was separated, the aqueous layer was extracted with CH₂Cl₂ (3×10 ml). The combined organic layers were washed with water (3×20 ml), dried over MgSO₄, and filtered. After removal of the solvent, the residue was dried in a vacuum oven to afford trioxolane OZ469 (497 mg, 92%) as a colorless solid. mp 129-131° C.; ¹H NMR (500 MHz, CDCl₃) δ 1.20-1.42 (m, 2H), 1.64-2.01 (m, 23H), 2.13 (d, J=6.8 Hz, 2H), 3.36-3.46 (m, 4H), 5.62-5.71 (m, 1H), 6.39 (d, J=5.4 Hz, 1H), 6.40-6.49 (m, 1H), 7.40 (dd, J=8.8, 2.4 Hz, 1H), 7.92 (d, J=8.3 Hz, 1H), 7.93 (d, J=2.0 Hz, 1H), 8.50 (d, J=5.4 Hz, 1H); ¹³C NMR (125.7 MHz, CDCl₃) δ 26.46, 26.84, 28.40, 30.03, 33.50, 33.93, 34.79, 36.30, 36.39, 36.77, 38.99, 43.68, 98.45, 108.41, 111.46, 117.59, 121.83, 125.36, 128.50, 134.91, 149.31, 149.84, 151.86, 173.46. Anal. Calcd for C₃₀H₃₈ClN₃O₄: C, 66.71; H, 7.09; N, 7.78. Found: C, 66.50; H, 6.89; N, 7.62.

cis-Adamantane-2-spiro-3′-8′-[[[[4′-[(7′-chloro-4′-quinolinyl)amino]butyl]amino]carbonyl]methyl]-1′,2′,4′-trioxaspiro[4.5]decane (OZ470). To a solution of N-(7-chloro-4-quinolinyl)-1,4-butanediamine (250 mg, 1.0 mmol) and triethylamine (202 mg, 2.0 mmol) in CH₂Cl₂ (10 ml) was added dropwise a solution of OZ78-HOBt active ester (439 mg, 1.0 mmol) in CH₂Cl₂ (10 ml) The mixture was stirred at rt for 1.5 h before it was quenched with water (20 ml). After the organic layer was separated, the aqueous layer was extracted with CH₂Cl₂ (3×10 ml). The combined organic layers were washed with water (3×20 ml), dried over MgSO₄, and filtered. After removal of the solvent, the residue was dried in a vacuum oven to afford trioxolane OZ470 (316 mg, 57%) as a colorless solid. mp 108-110° C.; ¹H NMR (500 MHz, CDCl₃) δ 1.15-1.32 (m, 2H), 1.64-2.01 (m, 25H), 2.06 (d, J=6.8 Hz, 2H), 3.31-3.42 (m, 4H), 5.45-5.55 (m, 1H), 5.61-5.69 (m, 1H), 6.39 (d, J=5.4 Hz, 1H), 7.36 (dd, J=8.8, 2.0 Hz, 1H), 7.81 (d, J=9.3 Hz, 1H), 7.94 (d, J=2.4 Hz, 1H), 8.51 (d, J=5.4 Hz, 1H); ¹³C NMR (125.7 MHz, CDCl₃) δ 25.73, 26.71, 27.09, 28.34, 30.24, 33.73, 34.21, 35.03, 36.63, 37.02, 39.18, 43.30, 43.82, 99.23, 108.73, 110.00, 111.68, 117.53, 121.62, 125.54, 128.94, 135.10, 149.41, 150.08, 152.23, 172.67. Anal. Calcd for C₃₁H₄₀ClN₃O₄: C, 67.19; H, 7.28; N, 7.58. Found: C, 67.17; H, 7.44; N, 7.39.

Adamantane-2-spiro-3′-5′-(3′-hydroxypropyl)-5′-methyl-1′,2′,4′-trioxolane (OZ471). To a solution of OZ467 (1.3 g, 4.2 mmol) in ether (10 ml) and THF (2 ml) was added dropwise 2 M lithium borohydride in THF (2.1 ml, 4.2 mmol) followed by 1 M lithium triethylborohydride in THF (0.42 ml, 0.42 mmol). The resulting mixture was stirred at rt overnight and was then diluted with ether (30 ml). The solvent mixture was washed with 1 M aq. NaOH (2×5 ml), water (2×5 ml) and brine (5 ml), dried over MgSO₄, filtered, and concentrated to afford trioxolane OZ471 as a colorless oil (0.85 g, 76%). ¹H NMR (500 MHz, CDCl₃) δ 1.43 (s, 3H), 1.62-2.02 (m, 18H), 3.65 (t, J=5.9 Hz, 2H); ¹³C NMR (125.7 MHz, CDCl₃) δ 23.08, 26.38, 26.76, 27.34, 34.23, 34.69, 34.72, 34.83, 36.10, 36.30, 36.70, 62.69, 109.83, 111.78. Anal. Calcd for C₁₅H₂₄O₄: C, 67.14; H, 9.01. Found: C, 66.90; H, 8.88.

5-Chloroadamantane-2-spiro-3′-1′,2′,4′-trioxaspiro[4.5]decane (OZ472). A solution of O-methyl cyclohexanone oxime (1.09 g, 8.57 mmol) and 5-chloro-2-adamantanone (1.05 g, 5.69 mmol) in cyclohexane (80 ml) and CH₂Cl₂ (20 ml) was treated with ozone according to the general procedure. After removal of the solvents, the crude product was purified by chromatography (silica gel, 10% ether in hexanes) followed by crystallization from methanol to afford trioxolane OZ472 (450 mg, 27%) as a colorless solid (2:1-mixture of two diastereomers). mp 57-59° C.; ¹H NMR (500 MHz, CDCl₃) δ 1.25-2.51 (m, 23H); ¹³C NMR (125.7 MHz, CDCl₃) δ 23.80, 24.88, 29.67, 30.07, 32.85, 32.94, 34.57, 34.65, 38.94, 39.18, 44.31, 44.37, 46.87, 46.93, 65.77, 66.22, 109.11, 109.15, 109.48, 109.57. Anal. Calcd for C₁₆H₂₃ClO₃: C, 64.31; H, 7.76. Found: C, 64.50; H, 7.51.

5-(p-Toluoyloxy)adamantane-2-spiro-3′-1′,2′,4′-trioxaspiro[4.5]decane (OZ473). A solution of O-methyl cyclohexanone oxime (1.35 g, 10.6 mmol) and 5-(p-toluoyloxy)-2-adamantanone (2.00 g, 7.05 mmol) in cyclohexane (80 ml) and CH₂Cl₂ (20 ml) was treated with ozone according to the general procedure. After removal of the solvents, the crude product was purified by chromatography (silica gel, 10% EtOAc in hexanes) followed by crystallization from methanol to afford trioxolane OZ473 (2.14 mg, 76%) as a colorless solid (2:1-mixture of two diastereomers). mp 79-81° C.; ¹H NMR (500 MHz, CDCl₃) δ 1.25-2.54 (m, 26H), 7.09-7.25 (m, 2H), 7.81-7.91 (m, 2H); ¹³C NMR (125.7 MHz, CDCl₃) δ 21.59, 23.84, 24.94, 28.89, 29.27, 33.42, 33.50, 34.62, 34.71, 38.15, 38.39, 38.41, 38.46, 40.10, 40.32, 78.67, 79.14, 109.38, 109.46, 109.78, 109.81, 128.88, 128.99, 129.47, 143.09, 143.10, 165.54. Anal. Calcd for C₂₄H₃₀O₅: C, 72.34; H, 7.59. Found: C, 72.27; H, 7.50.

5-Phenyladamantane-2-spiro-3′-1′,2′,4′-trioxaspiro[4.5]decane (OZ474). A solution of O-methyl cyclohexanone oxime (0.957 g, 7.52 mmol) and 5-phenyl-2-adamantanone (1.14 g, 5.02 mmol) in cyclohexane (80 ml) and CH₂Cl₂ (20 ml) was treated with ozone according to the general procedure. After removal of the solvents, the crude product was purified by chromatography (silica gel, 10% ether in hexanes) followed by crystallization from methanol to afford trioxolane OZ474 (750 mg, 44%) as a colorless solid (1:1-mixture of two diastereomers). mp 78-80° C.; ¹H NMR (500 MHz, CDCl₃) δ 1.21-2.41 (m, 23H), 7.15-7.45 (m, 5H); ¹³C NMR (125.7 MHz, CDCl₃) δ 23.87, 23.88, 24.98, 27.14, 27.53, 33.89, 33.99, 34.77, 34.80, 35.06, 35.46, 36.79, 36.89, 40.38, 42.20, 42.36, 109.17, 110.65, 124.84, 124.91, 125.76, 125.78, 128.14, 128.15, 149.58, 149.68. Anal. Calcd for C₂₂H₂₈O₃.0.2H₂O: C, 76.80; H, 8.32. Found: C, 76.43; H, 8.01.

5-Bromoadamantane-2-spiro-3′-1′,2′,4′-trioxaspiro[4.5]decane (OZ475). A solution of O-methyl cyclohexanone oxime (1.12 g, 8.84 mmol) and 5-bromo-2-adamantanone (1.35 g, 5.89 nmol) in cyclohexane (80 ml) and CH₂Cl₂ (20 ml) was treated with ozone according to the general procedure. After removal of the solvents, the crude product was purified by chromatography (silica gel, 10% ether in hexanes) followed by crystallization from methanol to afford trioxolane OZ475 (1.60 g, 79%) as a colorless solid (3:2-mixture of two diastereomers). mp 76-79° C.; ¹H NMR (500 MHz, CDCl₃) δ 1.25-2.78 (m, 23H); ¹³C NMR (125.7 MHz, CDCl₃) δ 23.77, 24.86, 30.46, 30.86, 32.80, 32.89, 34.55, 34.62, 39.78, 39.99, 45.81, 45.88, 48.33, 48.40, 62.17, 62.72, 108.89, 108.97, 109.46, 109.57. Anal. Calcd for C₁₆H₂₃BrO₃: C, 55.98; H, 6.75. Found: C, 55.81; H, 6.66.

5-Hydroxyadamantane-2-spiro-3′-1′,2′,4′-trioxaspiro[4.5]decane (OZ476). To a solution of OZ473 (0.90 g, 2.25 mmol) in ethanol (25 ml) and THF (25 ml) was added 1 M aq. NaOH (22.55 ml, 22.5 mmol). The reaction mixture was heated at 50° C. overnight and cooled to rt. The reaction mixture was diluted with water (50 ml) and extracted with CH₂Cl₂ (2×30 ml). The combined organic layer was dried over MgSO₄, filtered, and concentrated. The residue was purified by chromatography (silica gel, 10% EtOAc in hexanes) followed by crystallization from methanol to afford trioxolane OZ476 (130 mg, 21%) as a colorless solid (1:1-mixture of two diastereomers). mp 88-90° C.; ¹H NMR (500 MHz, CDCl₃) δ 1.25-2.21 (m, 23H); ¹³C NMR (125.7 MHz, CDCl₃) δ 23.83, 24.92, 28.77, 29.15, 33.32, 33.41, 34.61, 34.71, 38.02, 38.28, 42.03, 44.43, 62.30, 67.14, 67.56, 81.81, 81.89, 85.97, 97.85, 109.29, 109.35, 110.00. Calcd for C₁₆H₂₄O₄: C, 68.54; H, 8.63. Found: C, 68.38; H, 8.45.

cis-Adamantane-2-spiro-3′-8′-[[[[2′-[(7′-chloro-4′-quinolinyl)amino]ethyl]amino]carbonyl]methyl]-1′,2′,4′-trioxaspiro[4.5]decane (OZ477). To a solution of N-(7-chloro-4-quinolinyl)-1,2-ethanediamine (222 mg, 1.0 mmol) and triethylamine (202 mg, 2.0 mmol) in CH₂Cl₂ (10 ml) was added dropwise a solution of OZ78-HOBt active ester (439 mg, 1.0 mmol) in CH₂Cl₂ (10 ml) The mixture was stirred at rt for 1.5 h before it was quenched with water (20 ml). After the organic layer was separated, the aqueous layer was extracted with CH₂Cl₂ (3×10 ml). The combined organic layers were washed with water (3×20 ml), dried over MgSO₄, and filtered. After removal of the solvent, the residue was dried in a vacuum oven to afford trioxolane OZ477 (310 mg, 59%) as a colorless solid. mp 117-127° C.; ¹H NMR (500 MHz, CDCl₃) δ 1.15-1.28 (m, 2H), 1.55-2.01 (m, 21H), 2.12 (d, J=7.3 Hz, 2H), 3.36-3.42 (m, 2H), 3.68-3.76 (m, 2H), 5.95-6.12 (m, 1H), 6.28 (d, J=5.4 Hz, 1H), 6.61-6.69 (m, 1H), 7.39 (d, J=9.2 Hz, 1H), 7.76 (d, J=9.3 Hz, 1H), 7.93 (s, 1H), 8.48-8.52 (m, 1H); ¹³C NMR (125.7 MHz, CDCl₃) δ 26.45, 26.82, 29.90, 33.60, 33.81, 34.76, 36.34, 36.76, 38.79, 43.34, 46.07, 98.11, 108.30, 111.45, 117.18, 121.82, 125.49, 128.45, 134.93, 149.03, 150.03, 151.91, 175.18.

cis-Adamantane-2-spiro-3′-8′-(4′-nitrophenyl)-1′,2′,4′-trioxaspiro[4.5]decane (OZ478). Step 1. To a solution of 4-phenylcyclohexanone (6.96 g, 39.9 mmol) in acetic anhydride (60 ml) at −2° C. was added copper(II) nitrate (7.50 g, 31.0 mmol). After the mixture was stirred at −2° C. for 4 h, it was poured into water (200 ml). The mixture was extracted with CHCl₃ (3×50 ml). The combined organic layers were washed with water (50 ml) and saturated aq. NaHCO₃ (50 ml), and dried over MgSO₄. After the solvent was evaporated, the residue was crystallized from ethanol to give 4-(4-nitrophenyl)cyclohexanone (2.17 g, 25%) as a colorless solid. mp 160-162° C. (ethanol). ¹H NMR (500 MHz, CDCl₃) δ 1.91-2.09 (m, 2H), 2.20-2.29 (m, 2H), 2.47-2.62 (m, 4H), 3.12-3.22 (m, 1H), 7.42 (d, J=8.8 Hz, 2H), 8.20 (d, J=8.8 Hz, 2H); ¹³C NMR (125.7 MHz, CDCl₃) δ 33.8, 41.3, 42.9, 124.2, 127.9, 147.0, 152.6, 210.1. Step 2. A solution of O-methyl 2-adamantanone oxime (1.23 g, 6.84 mmol) and 4-(4-nitrophenyl)cyclohexanone (1.00 g, 4.56 mmol) in cyclohexane (130 ml) and CH₂Cl₂ (55 ml) was treated with ozone according to the general procedure. The crude product was crystallized from ethanol to give trioxolane OZ478 (1.12 g, 64%) as a colorless solid. mp 147-148° C.; ¹H NMR (500 MHz, CDCl₃) δ 1.62-2.17 (m, 22H), 2.61-2.72 (m, 1H), 7.36 (d, J=8.3 Hz, 2H), 8.15 (d, J=8.3 Hz, 2H); ¹³C NMR (125.7 MHz, CDCl₃) δ 26.45, 26.86, 31.10, 34.44, 34.79, 36.40, 36.76, 42.90, 107.89, 111.66, 123.75, 127.62, 146.52, 153.76. Anal. Calcd for C₂₂H₂₇NO₅: C, 68.55; H, 7.06; N, 3.63. Found: C, 69.17; H, 7.04; N, 3.42.

cis-Adamantane-2-spiro-3′-8′-(4′-aminophenyl)-1′,2′,4′-trioxaspiro[4.5]decane mesylate (OZ479). Step 1. To a solution of 4-(4-nitrophenyl)cyclohexanone (8.92 g, 40.7 mmol) in ethanol (160 ml) at 60° C. was added a solution of ammonium chloride (21.77 g, 407 mmol) in water (50 ml) followed by iron powder (6.82 g, 122 mmol) portionwise. The reaction mixture was refluxed for 3 h, cooled to rt, filtered, and extracted with EtOAc (2×100 ml). The combined organic layers were washed with brine and dried over MgSO₄. Removal of the solvent gave 4-(4-aminophenyl)cyclohexanone (4.83 g, 63%) as a yellow solid. mp 127-128° C. (ethanol); ¹H NMR (500 MHz, CDCl₃) δ 1.82-1.98 (m, 2H), 2.13-2.28 (m, 2H), 2.41-2.55 (m, 4H), 2.84-2.96 (m, 1H), 3.73 (brs, 2H), 6.66 (d, J=8.8 Hz, 2H), 7.04 (d, J=8.3 Hz, 2H); ¹³C NMR (125.7 MHz, CDCl₃) δ 34.5, 41.7, 42.2, 115.8, 127.7, 135.6, 144.4, 211.8. Step 2. To a solution of 4-(4-aminophenyl)cyclohexanone (1.00 g, 5.28 mmol) in toluene (30 ml) at 60° C. was added phthalic anhydride (0.78 g, 5.28 mmol). The reaction mixture was refluxed with a Dean-Stark adapter for 2 h and cooled to rt. After removal of the solvent, the residue was dissolved in CHCl₃, washed with saturated aq. NaHCO₃, and dried over MgSO₄. Removal of the solvent gave 4-(4-phthalimidophenyl)cyclohexanone (0.53 g, 31%) as a yellow solid. mp 150-152° C. (ethanol); ¹H NMR (500 MHz, CDCl₃) δ 1.89-2.05 (m, 2H), 2.21-2.34 (m, 2H), 2.44-2.59 (m, 4H), 3.09 (tt, J=12.2, 3.2 Hz, 1H), 7.39 (s, 4H), 7.76-7.82 (m, 2H), 7.93-7.98 (m, 2H); ¹³C NMR (125.7 MHz, CDCl₃) δ 34.1, 41.5, 42.7, 124.0, 126.9, 127.7, 130.3, 132.0, 134.7, 145.0, 167.6, 211.1. Step 3. A solution of O-methyl 2-adamantanone oxime (0.82 g, 4.55 mmol) and 4-(4-phthalimidophenyl)cyclohexanone (0.97 g, 3.0 mmol) in cyclohexane (80 ml) and CH₂Cl₂ (25 ml) was treated with ozone according to the general procedure. The crude product was crystallized from ethanol to afford the desired phthalimido-protected trioxolane (0.39 g, 27%) as a colorless solid. mp 146-147° C.; ¹H NMR (500 MHz, CDCl₃) δ 1.68-2.15 (m, 22H), 2.54-2.65 (m, 1H), 7.34 (s, 4H), 7.76-7.82 (m, 2H), 7.92-7.98 (m, 2H). Step 4. A mixture of the above phthalimido-protected trioxolane (0.39 g, 0.80 mmol) and hydrazine monohydrate (0.28 g, 5.70 mmol) in CHCl₃ (7 ml) and ethanol (3 ml) was heated to 60° C. overnight. After the reaction mixture was cooled to rt, a solid by-product was filtered off. The filtrate was diluted with CHCl₃, washed with water and brine, and dried over MgSO₄. After removal of the solvent, the residue was dissolved in ether (10 ml) and CHCl₃ (5 ml) and treated with a solution of methanesulfonic acid (0.04 g, 0.47 mmol) in ether (5 ml). The precipitate was collected by filtration to afford trioxolane OZ479 (0.67 g, 70%) as a colorless solid. mp 140-142° C.; ¹H NMR (500 MHz, CDCl₃) δ 1.62-2.11 (m, 22H), 2.48-2.59 (m, 1H), 2.64 (s, 3H), 7.21 (d, J=8.3 Hz, 2H), 7.42 (d, J=8.3 Hz, 2H), 9.73 (brs, 3H); ¹³C NMR (125.7 MHz, CDCl₃) δ 26.48, 26.88, 31.34, 34.54, 34.79, 36.40, 36.80, 39.15, 42.40, 108.14, 111.48, 123.43, 128.09, 128.74, 146.89. Anal. Calcd for C₂₃H₃₃NO₆S: C, 61.17; H, 7.37; N, 3.10. Found: C, 55.96; H, 7.00; N, 3.66.

cis-Adamantane-2-spiro-3′-8′-[3′-[2′-(4′-morpholinyl)ethoxy]phenyl]-1′,2′,4′-trioxaspiro[4.5]decane mesylate (OZ480). To a solution of OZ454 (0.13 g, 0.37 mmol) in dry acetonitrile (15 ml) were added powered NaOH (0.10 g, 2.50 mmol) and tetrabutylammonium hydrogensulfate (0.02 g, 0.07 mmol). The mixture was stirred at rt for 30 min before N-(2-chloroethyl)morpholine hydrochloride (0.20 g, 1.08 mmol) was added. The reaction mixture was stirred at 60° C. overnight, cooled to rt, filtered, and washed with CH₂Cl₂. After the filtrate was concentrated, the residue was dissolved in CH₂Cl₂ (20 ml), washed with water (2×10 ml) and brine (10 ml), and dried over MgSO₄, filtered, and concentrated. The crude product was purified by chromatography (50% EtOH in EtOAc) to afford the desired free base (0.13 g). To a solution of the free base in CH₂Cl₂ (5 ml) was added a solution of methanesulfonic acid (0.03 g, 0.31 mmol) in ether (20 ml). The precipitate was collected by filtration to afford trioxolane OZ480 (0.12 g, 57%) as a colorless solid. mp 146-147° C.; ¹H NMR (500 MHz, CDCl₃) δ 1.59-2.11 (m, 22H), 2.48-2.58 (m, 1H), 2.83 (s, 3H), 3.02-3.14 (m, 2H), 3.51-3.59 (m, 2H), 3.64-3.72 (m, 2H), 3.98-4.18 (m, 4H), 4.44-4.53 (m, 2H), 6.71-6.76 (m, 2H), 6.88 (d, J=7.8 Hz, 1H), 7.23 (t, J=7.8 Hz, 1H), 11.81 (brs, 1H); ¹³C NMR (125.7 MHz, CDCl₃) δ 26.48, 26.87, 31.36, 34.61, 34.80, 36.40, 36.79, 39.37, 42.86, 52.98, 56.90, 62.74, 63.86, 108.27, 111.51, 111.68, 113.45, 120.71, 129.78, 148.45, 157.11. Anal. Calcd for C₂₉H₄₃NO₈S: C, 61.57; H, 7.66; N, 2.48. Found: C, 61.31; H, 7.43; N, 2.34.

cis-Adamantane-2-spiro-3′-8′-[4′-[3′-(4′-acetyl-1′-piperazinyl)propoxy]phenyl]-1′,2′,4′-trioxaspiro[4.5]decane p-tosylate (OZ481). Step 1. To a suspension of 1-(3-chloropropyl)piperazine dihydrochloride (2.00 g, 8.18 mmol) in CH₂Cl₂ (50 ml) at 0° C. was added dropwise triethylamine (11.4 ml, 81.8 mmol) followed by a solution of acetyl chloride (1.2 ml, 16.4 mmol) in CH₂Cl₂ (10 ml). The reaction mixture was stirred at rt overnight and quenched with water. The organic layer was washed with water (3×25 ml), dried over MgSO₄, and filtered. After removal of the solvent under vacuum, the residue was purified by chromatography (silica gel, 50% EtOH in EtOAc) to afford 1-acetyl-4-(3-chloropropyl)piperazine (1.20 g, 72%) as an oil. ¹H NMR (500 MHz, CDCl₃) δ 1.88-1.94 (m, 2H), 2.05 (s, 3H), 2.37 (t, J=5.4 Hz, 2H), 2.41 (t, J=5.1 Hz, 2H), 2.47 (t, J=6.8 Hz, 2H), 3.42-3.44 (m, 2H), 3.56-3.59 (m, 4H). Step 2. To a solution of OZ288 (0.50 g, 1.4 mmol) in dry acetonitrile (50 ml) were added powdered NaOH (0.225 g, 5.61 mmol) and tetrabutylammonium hydrogensulfate (0.095 g, 0.28 mmol). After the reaction mixture was stirred at rt for 30 min, 1-acetyl-4-(3-chloropropyl)piperazine (0.43 g, 2.1 mmol) was added. The reaction mixture was stirred at 60° C. overnight and cooled to rt. The inorganic solid was filtered off and washed with EtOAc (2×25 ml). After removal of the solvents under vacuum, the residue was dissolved in EtOAc (50 ml). The organic layer was washed with water and brine, dried over MgSO₄, and filtered. After removal of the solvent, the residue was purified by chromatography (silica gel, 50% EtOH in EtOAc) to afford the desired free base (0.30 g, 41%). To a solution of the above free base (0.30 g, 0.57 mmol) in EtOAc (10 ml) at 0° C. was added dropwise a solution of p-toluenesulfonic acid monohydrate (0.11 g, 0.57 mmol) in ether (10 ml). The resulting precipitate was filtered, washed with ether (25 ml), and dried under vacuum at 40° C. to afford trioxolane OZ481 (0.35 g, 86%) as a colorless solid. mp 152-154° C.; ¹H NMR (500 MHz, DMSO-d₆) δ 1.48-1.58 (m, 2H), 1.62-1.96 (m, 20H), 2.05 (s, 3H), 2.07-2.15 (m, 2H), 2.29 (s, 3H), 2.48-2.59 (m, 1H), 2.84-2.99 (m, 1H), 3.02-3.15 (m, 1H), 3.21-3.32 (m, 2H), 3.33-3.46 (m, 2H), 3.49-3.59 (m, 2H), 3.95-4.08 (m, 3H), 4.44 (d, J=11.2 Hz, 1H), 6.85 (d, J=8.8 Hz, 2H), 7.09-7.15 (m, 4H), 7.50 (d, J=7.8 Hz, 2H), 9.54 (brs, 1H); ¹³C NMR (125.7 MHz, DMSO-d₆) δ 20.95, 21.13, 23.64, 25.99, 26.40, 31.48, 34.27, 34.43, 35.96, 36.26, 38.03, 40.86, 42.80, 51.00, 51.29, 53.48, 64.82, 108.30, 110.72, 114.58, 125.66, 127.65, 128.28, 137.94, 138.47, 145.67, 156.65, 168.76. Anal. Calcd for C₃₈H₅₂N₂O₈S: C, 65.49; H, 7.52; N, 4.02. Found: C, 65.50; H, 7.77; N, 3.83.

Adamantane-2-spiro-3′-1′,2′,4′-trioxaspiro[4.7]dodecane (OZ483). A solution of O-methyl cyclooctanone oxime (1.55 g, 10 mmol) and 2-adamantanone (3.0 g, 20 mmol) in DCM (10 ml) and pentane (90 ml) was treated with ozone according to the general procedure. The reaction solution was then concentrated to dryness. The residue was purified by flash chromatography (silica gel, 1% ethyl ether in hexane) three times to afford trioxolane OZ483 (105 mg, 4%) as a colorless oil. ¹H NMR (500 MHz, CDCl₃) δ 1.48-2.06 (m, 28H); ¹³C NMR (125.7 MHz, CDCl₃) δ 22.30, 24.56, 26.51, 26.92, 27.69, 33.99, 34.76, 34.84, 36.32, 36.82, 111.36, 112.35.

cis-Adamantane-2-spiro-3′-8′-[2′-[[2′-[(7′-chloro-4′-quinolinyl)amino]ethyl]amino]ethyl]-1′,2′,4′-trioxaspiro[4.5]decane (OZ484). Step 1. To a solution of the OZ78 methyl ester (4.00 g, 11.9 mmol) in CH₂Cl₂ (20 ml) at −78° C. was added DIBAL-H (17.8 ml, 1 M CH₂Cl₂ solution, 17.8 mmol). The reaction mixture was stirred at −78° C. for 2.5 h before it was quenched with saturated aq. NH₄Cl (2 ml). The mixture was warmed to rt, diluted with CH₂Cl₂ (30 ml), washed water (2×50 ml), dried over MgSO₄, and concentrated. The residue was purified by chromatography (silica gel, 10% ether in hexane) to give the desired aldehyde (1.45 g, 40%). ¹H NMR (500 MHz, CDCl₃) δ 1.20-1.35 (m, 2H), 1.51-2.05 (m, 21H), 2.34 (dd, J=6.8, 2.0 Hz, 2H), 9.75 (t, J=2.0 Hz, 1H). Step 2. To a solution of the above aldehyde (0.556 g, 1.8 mmol) in 1,2-dichloroethane (10 ml) was added a solution of N-(7-chloro-4-quinolinyl)-1,2-ethanediamine (0.498 g, 2.2 mmol) in CH₂Cl₂ (10 ml) followed by acetic acid (1 ml). After the mixture was stirred at rt for 2 h, sodium triacetoxyborohydride (0.616 g, 2.9 mmol) was added. The reaction mixture was stirred at rt overnight and then quenched with 1.0 M aq. NaOH (10 ml). After separation of the organic layer, the aqueous layer was extracted with CH₂Cl₂ (2×10 ml). The combined organic layers were washed with water (50 ml), dried over MgSO₄, and concentrated. The residue was purified by crystallization from ether to afford trioxolane OZ484 (0.13 g, 14%) as a colorless solid. mp 142-143° C.; ¹H NMR (500 MHz, CDCl₃) δ 1.19-2.07 (m, 25H), 2.71 (t, J=7.3 Hz, 2H), 3.06 (t, J=5.6 Hz, 2H), 3.37 (t, J=5.6 Hz, 2H), 6.36 (d, J=5.4 Hz, 1H), 7.38 (dd, J=8.8, 2.0 Hz, 1H), 7.77 (d, J=8.8 Hz, 1H), 7.94 (d, J=2.0 Hz, 1H), 8.49 (d, J=5.4 Hz, 1H); ¹³C NMR (125.7 MHz, CDCl₃) δ 26.48, 26.88, 30.17, 34.19, 34.79, 34.81, 36.40, 36.57, 36.80, 41.93, 47.03, 47.53, 99.23, 108.78, 111.29, 117.37, 121.17, 125.31, 128.79, 134.85, 149.15, 149.85, 152.10. Anal. Calcd for C₂₉H₃₈ClN₃O₃: C, 68.02; H, 7.48; N, 8.21. Found: C, 67.88; H, 7.31; N, 8.16.

cis-Adamantane-2-spiro-3′-8′-(2′-hydroxyphenyl)-1′,2′,4′-trioxaspiro[4.5]decane (OZ486). Step 1. To a 250 ml round-bottom flask equipped with a stirrer, condenser, and addition funnel were added magnesium turnings (0.92 g, 38 mmol) and enough THF to cover the Mg. A solution of 2-benzyloxy-1-bromobenzene (5.0 g, 19 mmol) in THF (50 ml) was added dropwise at such a rate that the reaction maintained a gentle reflux. After the mixture was refluxed for an additional 2 h, a solution of 1,4-cyclohexanedione monoethylene ketal (3.0 g, 19 mol) in THF (60 ml) was added dropwise. The mixture was kept at refluxing overnight before being quenched with saturated ammonium chloride solution (10 ml). After removal of magnesium salts by filtration, the filtrate was concentrated to dryness. The residue was dissolved in CH₂Cl₂ (100 ml) and washed with water (2×50 ml) and brine (50 ml). The organic layer was separated, dried over MgSO₄, filtered, and concentrated. The crude product was purified by chromatography (silica gel, 30% ether in hexane) to afford the desired alcohol (3.80 g, 58%) as a colorless solid. mp 103-104° C.; ¹H NMR (500 MHz, CDCl₃) δ 1.64-1.76 (m, 2H), 2.10-2.26 (m, 6H), 3.93-4.03 (m, 4H), 5.17 (s, 2H), 6.96-7.05 (m, 2H), 7.21-7.29 (m, 1H), 7.34-7.50 (m, 6H); ¹³C NMR (125.7 MHz, CDCl₃) δ 30.28, 34.07, 63.97, 64.15, 70.30, 72.06, 108.58, 112.37, 121.20, 125.76, 127.26, 128.06, 128.12, 128.69, 135.30, 136.10, 156.27. Step 2. To a solution of the above alcohol (1.40 g, 4.12 mmol) and triethylamine (3.0 ml, 21.6 mmol) in CH₂Cl₂ (50 ml) at 0° C. was added dropwise methanesulfonyl chloride (0.64 ml, 8.27 mmol). After being stirred at 0° C. for 1 h and at rt overnight, the reaction mixture was washed with water (2×20 ml) and brine (20 ml), dried over MgSO₄, filtered, and concentrated. The crude product was purified by chromatography (silica gel, 10% to 30% ether in hexane) to afford the desired olefin (0.80 g, 60%) as a colorless solid. mp 84-85° C.; ¹H NMR (500 MHz, CDCl₃) δ 1.87 (t, J=7.0 Hz, 2H), 2.43-2.49 (m, 2H), 2.64-2.70 (m, 2H), 3.98-4.08 (m, 4H), 5.09 (s, 2H), 5.65-5.71 (m, 1H), 6.89-6.95 (m, 2H), 7.16-7.23 (m, 2H), 7.27-7.33 (m, 1H), 7.34-7.40 (m, 2H), 7.41-7.46 (m, 2H); ¹³C NMR (125.7 MHz, CDCl₃) δ 28.08, 31.43, 36.22, 64.39, 70.25, 107.91, 112.71, 120.99, 123.37, 126.86, 127.57, 128.00, 128.41, 129.71, 133.21, 136.89, 137.41, 155.84. Step 3. To a solution of the above olefin (0.90 g, 2.8 mmol) in EtOAc (50 ml) was added 10% Pd/carbon (0.1 g). The resulting mixture was hydrogenated for 24 h at atmospheric pressure. After the mixture was filtered through Celite, the filtrate was concentrated to afford the desired phenol (0.60 g, 92%) as a colorless solid. mp 92-93° C.; ¹H NMR (500 MHz, CDCl₃) δ 1.60-1.96 (m, 8H), 2.86-2.98 (m, 1H), 3.99 (s, 4H), 6.71-6.76 (m, 1H), 6.86-6.92 (m, 1H), 7.03-7.10 (m, 1H), 7.19-7.24 (m, 1H); ¹³C NMR (125.7 MHz, CDCl₃) δ 29.98, 35.22, 35.90, 64.25, 64.29, 108.72, 115.17, 120.90, 126.79, 127.02, 132.37, 152.82. Step 4. A mixture of the above phenol (1.10 g, 4.70 mmol) in EtOAc (10 ml) was added to a solution of dry pyridine (20 ml) and acetic anhydride (8 ml) at −70° C., and the reaction mixture was stored at −30° C. overnight. After removal of the solvents, the residue was partitioned between CH₂Cl₂ (30 ml) and water (30 ml). The aqueous layer was extracted with CH₂Cl₂ (2×30 ml). The combined extracts were washed with 1 M aq. HCl (2×10 ml) and water (10 ml), dried over MgSO₄, filtered, and concentrated to afford the desired ketal ester (1.27 g, 98%) as a colorless oil. ¹H NMR (500 MHz, CDCl₃) δ 1.60-1.90 (m, 8H), 2.34 (s, 3H), 2.60-2.70 (m, 1H), 3.98 (s, 4H), 6.98-7.02 (m, 1H), 7.18-7.24 (m, 2H), 7.30-7.36 (m, 1H); ¹³C NMR (125.7 MHz, CDCl₃) δ 20.94, 30.36, 35.25, 36.81, 64.31, 108.35, 122.31, 126.32, 126.90, 127.33, 137.91, 148.20, 169.75. Step 5. A mixture of the above ketal (1.25 g, 4.53 mmol), PPTS (0.1 g) in acetone (20 ml) and water (4 ml) was refluxed for 2 d. After removal of acetone, the residue was partitioned between CH₂Cl₂ (30 ml) and water (30 ml). The aqueous layer was extracted with CH₂Cl₂ (2×30 ml). The combined extracts were washed with water (2×20 ml) and brine (20 ml), dried over MgSO₄, filtered, and concentrated to afford 4-(2-acetoxyphenyl)cyclohexanone (1.0 g, 95%) as a colorless solid. mp 86-87° C.; ¹H NMR (500 MHz, CDCl₃) δ 1.84-1.98 (m, 2H), 2.13-2.22 (m, 2H), 2.37 (s, 3H), 2.43-2.57 (m, 4H), 3.06-3.16 (m, 1H), 7.02-7.08 (m, 1H), 7.20-7.31 (m, 3H); ¹³C NMR (125.7 MHz, CDCl₃) δ 21.01, 32.85, 36.33, 41.46, 122.58, 126.51, 126.80, 127.46, 136.32, 148.22, 169.59, 210.72. Step 6. A solution of O-methyl 2-adamantanone oxime (1.50 g, 8.38 mmol) and 4-(2-acetoxyphenyl)cyclohexanone (1.30 g, 5.60 mmol) in cyclohexane (120 ml) and CH₂Cl₂ (40 ml) was treated with ozone according to the general procedure. After removal of the solvents, the crude product was purified by crystallization from EtOH to afford cis-adamantane-2-spiro-3′-8′-(2′-acetoxyphenyl)-1′,2′,4′-trioxaspiro[4.5]decane (1.32 g, 59%) as a colorless solid. mp 118-119° C.; ¹H NMR (500 MHz, CDCl₃) δ 1.66-2.09 (m, 22H), 2.33 (s, 3H), 2.58-2.68 (m, 1H), 6.96-7.02 (m, 1H), 7.07-7.23 (m, 2H), 7.24-7.30 (m, 1H); ¹³C NMR (125.7 MHz, CDCl₃) δ 20.91, 26.47, 26.88, 30.24, 34.79, 34.82, 34.85, 36.41, 36.57, 36.79, 108.23, 111.44, 122.33, 126.41, 127.02, 127.22, 137.52, 148.15, 169.69. Step 7. To a solution of cis-adamantane-2-spiro-3′-8′-(2′-acetoxyphenyl)-1′,2′,4′-trioxaspiro[4.5]decane (0.40 g, 1.0 mmol) in MeOH (8 ml) and THF (4 ml) was added 15% aq. KOH solution (1.3 ml). The resulting mixture was stirred at 50° C. for 4 h. The solution was concentrated to ˜5 ml and the residue was diluted with water (10 ml) and acidified with acetic acid (1 ml). The precipitate was collected by filtration, washed with cold water, and dried in a vacuum oven at 40° C. to afford trioxolane OZ486 (0.33 g, 94%) as a colorless solid. mp 119-120° C.; ¹H NMR (500 MHz, CDCl₃) δ 1.62-2.17 (m, 22H), 2.82-2.98 (m, 1H), 4.72 (brs, 1H), 6.72 (d, J=7.8 Hz, 1H), 6.90 (t, J=7.6 Hz, 1H), 7.06 (td, J=7.8, 1.5 Hz, 1H), 7.16 (d, J=7.8 Hz, 1H); ¹³C NMR (125.7 MHz, CDCl₃) δ 26.50, 26.90, 29.89, 33.21, 34.81, 34.85, 35.59, 36.42, 36.82, 108.56, 111.36, 115.20, 121.08, 126.90, 126.95, 132.09, 152.66. Anal. Calcd for C₂₂H₂₈O₄: C, 74.13; H, 7.92. Found: C, 75.16; H, 8.36.

cis-Adamantane-2-spiro-3′-8′-[4′-[2′-(4′-acetyl-1′-piperazinyl)ethoxy]phenyl]-1′,2′,4′-trioxaspiro[4.5]decane p-tosylate (OZ487). Step 1. To a solution of N-(2-chloroethyl)piperazine dihydrochloride (1.50 g, 6.82 mmol) and triethylamine (4.75 ml, 34.1 mmol) in 1,2-dichloroethane (50 ml) at 0° C. was added dropwise a solution of acetyl chloride (0.54 gm, 6.82 mmol) in 1,2-dichloroethane (10 ml). After the addition, the reaction mixture was stirred at rt overnight and then quenched with water (25 ml). The organic layer was washed with water (3×25 ml) and dried over MgSO₄. After the solvent was removed under vacuum, the residue was purified by chromatography (silica gel, 50% EtOH in EtOAc) to afford 1-acetyl-4-(2-chloroethyl)piperazine (0.26 g, 20%) as a viscous oil. Step 2. To a solution of OZ288 (0.50 g, 1.40 mmol) in dry acetonitrile (50 ml) were added powdered NaOH (0.17 g, 4.21 mmol) and tetrabutylammonium hydrogensulfate (0.1 g, 0.28 mmol). After the reaction mixture was stirred at rt for 30 min, a solution of 1-acetyl-4-(2-chloroethyl)piperazine (0.26 g, 1.37 mmol) in acetonitrile (5 ml) was added. After the reaction mixture was stirred at 60° C. overnight and cooled to rt, the inorganic solid was filtered off and washed with EtOAc (2×25 ml). After removal of the solvent under vacuum, the residue was dissolved in EtOAc (50 ml). The organic layer was washed with water and brine and dried over MgSO₄. After removal of the solvent under vacuum, the residue was dissolved in EtOAc (20 ml) and treated with a solution of p-toluenesulfonic acid monohydrate (0.27 g, 1.40 mmol) in ether (5 ml) at 0° C. The resulting precipitate was filtered, washed with ether (3×10 ml), and dried under vacuum at 40° C. to afford trioxolane OZ487 (0.18 g, 18%) as a colorless solid. mp 148-150° C.; ¹H NMR (500 MHz, DMSO-d₆) δ 1.48-1.59 (m, 2H), 1.61-1.96 (m, 20H), 2.04 (s, 3H), 2.29 (s, 3H), 2.52-2.62 (m, 1H), 2.89-3.61 (m, 8H), 4.02 (d, J=14.2 Hz, 1H), 4.31 (s, 2H), 4.42 (d, J=12.7 Hz, 1H), 6.92 (d, J=7.8 Hz, 2H), 7.11 (d, J=7.3 Hz, 2H), 7.16 (d, J=7.8 Hz, 2H), 7.49 (d, J=7.3 Hz, 2H), 9.84 (brs, 1H); ¹³C NMR (125.7 MHz, DMSO-d₆) δ 20.95, 21.11, 25.99, 26.40, 31.47, 34.26, 34.43, 35.97, 36.26, 37.91, 40.88, 42.67, 51.45, 51.77, 54.97, 62.20, 108.29, 110.73, 114.85, 125.66, 127.73, 128.27, 137.90, 139.11, 145.71, 155.98, 168.73. Anal. Calcd for C₃₇H₅₀N₂O₈S: C, 65.08; H, 7.38; N, 4.10. Found: C, 65.35; H, 7.37; N, 3.89.

cis-Adamantane-2-spiro-3′-8′-[4′-[2′-(4′-trifluoromethyl-1′-piperidinyl)ethoxy]phenyl]-1′,2′,4′-trioxaspiro[4.5]decane mesylate (OZ488). Step 1. To the stirred mixture of 4-(trifluoromethyl)piperidine hydrochloride (1.00 g, 5.29 mmol) and potassium carbonate (3.70 g, 26.45 mmol) in acetonitrile at rt was added 2-bromoethanol (1.98 g, 15.87 mmol). After the reaction mixture was refluxed for 5 h, cooled to rt, and filtered, the filtrate was concentrated under vacuum. The residue was dissolved in 1,2-dichloroethane (50 ml) and thionyl chloride (5 ml) was added at rt. The reaction mixture was refluxed overnight, cooled to rt, and concentrated. The residue was triturated with ether (50 ml). The resulting precipitate was filtered, washed with ether (3×25 ml), and dried at 50° C. to afford 1-(2-chloroethyl)-4-(trifluoromethyl)piperidine hydrochloride (1.15 g, 87%) as a colorless solid. ¹H NMR (500 MHz, CDCl₃) δ 2.02-2.64 (m, 5H), 2.72-3.9 (m, 5H), 3.98-4.2 (m, 3H), 12.60 (brs, 1H). Step 2. To a solution of OZ288 (0.6 g, 1.69 mmol) in dry acetonitrile (50 ml) were added powdered NaOH (0.27 g, 6.74 mmol) and tetrabutylammonium hydrogensulfate (0.11 g, 0.34 mmol). After the reaction mixture was stirred at rt for 30 min, 1-(2-chloroethyl)-4-(trifluoromethyl)piperidine hydrochloride (0.85 g, 3.37 mmol) was added. The reaction mixture was stirred at 60° C. overnight and cooled to rt. The inorganic solid was filtered off and washed with EtOAc (2×25 ml). After removal of the solvent under vacuum, the residue was dissolved in EtOAc (50 ml). The organic layer was washed with water and brine and dried over MgSO₄. The solvent was removed under vacuum and the residue was purified by chromatography (silica gel, 50% EtOH in EtOAc-EtOH) to afford the desired free base as a colorless solid. ¹H NMR (500 MHz, CDCl₃) δ 1.61-2.12 (m, 30H), 2.46-2.52 (m, 1H), 2.79 (t, J=5.9 Hz, 2H), 3.07-3.09 (m, 1H), 4.07 (t, J=5.6 Hz, 2H), 6.83 (d, J=8.3 Hz, 2H), 7.11 (d, J=8.3 Hz, 2H); ¹³C NMR (125.7 MHz, CDCl₃) δ 24.63 (q, J=2.8 Hz), 26.48, 26.88, 31.63, 34.73, 34.79, 36.4, 36.79, 40.21 (q, J=27.0 Hz), 42.04, 52.84, 57.22, 65.98, 108.41, 111.35, 114.45, 127.46 (q, J=278.5 Hz), 127.61, 138.55, 157.04. To the solution of the above free base in EtOAc (10 ml) at 0° C. was added dropwise a solution of methanesulfonic acid (0.16 g, 1.69 mmol) in ether (10 ml). The resulting precipitate was filtered, washed with ether (3×10 ml), and dried under vacuum at 40° C. to afford trioxolane OZ488 (0.70 g, 66%) as a colorless solid. mp 155-157° C.; ¹H NMR (500 MHz, CDCl₃) δ 1.59-2.24 (m, 26H), 2.29-2.43 (m, 1H), 2.45-2.57 (m, 1H), 2.78 (s, 3H), 2.82-3.05 (m, 2H), 3.56 (s, 2H), 3.84 (d, J=11.7 Hz, 2H), 4.44 (s, 2H), 6.83 (d, J=8.3 Hz, 2H), 7.13 (d, J=8.3 Hz, 2H), 11.20 (brs, 1H); ¹³C NMR (125.7 MHz, CDCl₃) δ 22.24, 26.43, 26.82, 31.53, 34.61, 34.75, 36.36, 36.74, 38.10 (q, J=29.3 Hz), 39.37, 41.94, 49.40, 51.98, 56.31, 62.68, 108.27, 111.38, 114.42, 126.05 (q, J=278.5 Hz), 127.93, 139.93, 155.37. Anal. Calcd for C₃₁H₄₄F₃NO₇S: C, 58.94; H, 7.02; N, 2.22. Found: C, 58.98; H, 7.21; N, 2.07.

cis-Adamantane-2-spiro-3′-8′-[4′-[2′-(4′,4′-difluoro-1′-piperidinyl)ethoxy]phenyl]-1′,2′,4′-trioxaspiro[4.5]decane mesylate (OZ489). Step 1. To a mixture of 4,4-difluoropiperidine hydrochloride (1.00 g, 6.37 mmol) and potassium carbonate (4.40 g, 31.48 mmol) in acetonitrile (50 ml) at rt was added 2-bromoethanol (1.59 g, 12.74 mmol). The reaction mixture was refluxed for 5 h and cooled to rt. The reaction mixture was filtered and the filtrate was concentrated under vacuum. The residue was dissolved in 1,2-dichloroethane (50 ml) and thionyl chloride (5 ml) was added at rt. The reaction mixture was refluxed overnight and cooled to rt. After the solvent was removed under vacuum, the residue was triturated with ether (50 ml). The resulting solid was filtered, washed with ether (3×25 ml), and dried at 50° C. to afford 1-(2-chloroethyl)-4,4-difluoropiperidine hydrochloride (1.20 g, 86%) as a colorless solid. ¹H NMR (500 MHz, CDCl₃) δ 2.26-2.38 (m, 2H), 2.78-2.96 (m, 2H), 3.06-3.18 (m, 2H), 3.47 (t, J=6.1 Hz, 2H), 3.7-3.84 (m, 2H), 4.11 (t, J=6.1 Hz, 2H). Step 2. To a solution of OZ288 (0.60 g, 1.69 mmol) in dry acetonitrile (50 ml) were added powdered NaOH (0.27 g, 6.74 mmol) and tetrabutylammonium hydrogensulfate (0.11 g, 0.34 mmol). After the reaction mixture was stirred at rt for 30 min, 1-(2-chloroethyl)-4,4-difluoropiperidine hydrochloride (0.74 g, 3.37 mmol) was added. The reaction mixture was stirred at 60° C. overnight and cooled to rt. The inorganic solid was filtered off and washed with EtOAc (2×25 ml). After removal of the solvent under vacuum, the residue was purified by crystallization from EtOAc to afford the desired free base (0.65 g, 77%) as a colorless solid. ¹H NMR (500 MHz, CDCl₃) δ 1.66-2.04 (m, 26H), 2.47-2.52 (m, 1H), 2.67-2.69 (m, 4H), 2.84 (t, J=5.6 Hz, 2H), 4.06 (t, J=5.6 Hz, 2H), 6.83 (d, J=8.8 Hz, 2H), 7.11 (d, J=8.8 Hz, 2H); ¹³C NMR (125.7 MHz, CDCl₃) δ 26.47, 26.87, 31.62, 33.95 (t, J=23.1 Hz), 34.71, 34.79, 36.39, 36.78, 42.03, 50.41 (t, J=5.3 Hz), 56.35, 66.11, 108.39, 111.34, 114.44, 121.84 (t, J=241.4 Hz), 127.61, 138.61, 156.99. To a solution of the above free base (0.65 g, 1.29 mmol) in EtOAc (10 ml) at 0° C. was added dropwise a solution of methanesulfonic acid (0.12 g, 1.29 mmol) in ether (10 ml). The resulting precipitate was filtered, washed with ether (3×10 ml), and dried under vacuum at 40° C. to afford trioxolane OZ489 (0.65 g, 84%) as a colorless solid. mp 158-160° C.; ¹H NMR (500 MHz, CDCl₃) δ 1.59-2.08 (m, 22H), 2.21-2.35 (m, 2H), 2.45-2.57 (m, 1H), 2.56-2.74 (m, 2H), 2.79 (s, 3H), 3.13-3.27 (m, 2H), 3.59 (brs, 2H), 3.77-3.88 (m, 2H), 4.45 (brs, 2H), 6.84 (d, J=7.8 Hz, 2H), 7.14 (d, J=8.3 Hz, 2H), 11.59 (brs, 1H); ¹³C NMR (125.7 MHz, CDCl₃) δ 26.39, 26.78, 31.07 (t, J=25.9 Hz), 31.49, 34.57, 34.71, 36.31, 36.70, 39.27, 41.92, 50.32, 50.40, 55.78, 62.77, 108.22, 111.33, 114.40, 118.37 (t, J=243.2 Hz), 127.91, 139.97, 155.29. Anal. Calcd for C₃₀H₄₃F₂NO₇S: C, 60.08; H, 7.23; N, 2.34. Found: C, 60.01; H, 7.15; N, 2.09.

cis-Adamantane-2-spiro-3′-8′-[2′-[2′-(4′-morpholinyl)ethoxy]phenyl]-1′,2′,4′-trioxaspiro[4.5]decane mesylate (OZ490). To a solution of OZ486 (0.23 g, 0.65 mmol) in dry acetonitrile (15 ml) were added powered NaOH (0.16 g, 4.0 mmol) and tetrabutylammonium hydrogensulfate (0.04 g, 0.12 mmol). The mixture was stirred at rt for 30 min before N-(2-chloroethyl)morpholine hydrochloride (0.18 g, 0.97 mmol) was added. The reaction mixture was stirred at 60° C. overnight, cooled to rt, filtered, and washed with CH₂Cl₂. After the filtrate was concentrated, the residue was dissolved in CH₂Cl₂ (20 ml), washed with water (2×10 ml) and brine (10 ml), and dried over MgSO₄, filtered, and concentrated. The residue was dissolved in CH₂Cl₂ (5 ml) and then a solution of methanesulfonic acid (0.06 g, 0.63 mmol) in ethyl acetate (20 ml) was added. The precipitate was collected by filtration to afford trioxolane OZ490 (0.14 g, 38%) as a colorless solid. mp 148-149° C.; ¹H NMR (500 MHz, DMSO-d₆) δ 1.48-1.59 (m, 2H), 1.61-1.96 (m, 20H), 2.32 (brs, 3H), 2.92-3.01 (m, 1H), 3.22-3.41 (m, 2H), 3.54 (d, J=12.7 Hz, 2H), 3.62 (brs, 2H), 3.70 (t, J=11.7 Hz, 2H), 4.03 (d, J=11.7 Hz, 2H), 4.34 (brs, 2H), 6.91-7.03 (m, 2H), 7.15-7.24 (m, 2H), 9.87 (brs, 1H); ¹³C NMR (125.7 MHz, DMSO-d₆) δ 25.97, 26.41, 30.03, 34.45, 34.68, 35.98, 36.25, 52.10, 55.67, 63.10, 63.59, 108.41, 110.72, 112.14, 121.63, 126.32, 127.26, 133.92, 154.91. Anal. Calcd for C₂₉H₄₃NO₈S: C, 61.57; H, 7.66; N, 2.48. Found: C, 61.39; H, 7.74; N, 2.29.

cis-Adamantane-2-spiro-3′-8′-[2′-(3′-aminopropoxy)phenyl]-1′,2′,4′-trioxaspiro[4.5]decane mesylate (OZ491). To a solution of OZ486 (0.25 g, 0.70 mmol) in dry acetonitrile (15 ml) were added powered NaOH (0.17 g, 4.25 mmol) and tetrabutylammonium hydrogensulfate (0.05 g, 0.14 mmol). The mixture was stirred at rt for 30 min before 3-chloropropylamine hydrochloride (0.28 g, 2.1 mmol) was added. The reaction mixture was stirred at 60° C. overnight, cooled to rt, filtered, and washed with CH₂Cl₂. After the filtrate was concentrated, the residue was dissolved in CH₂Cl₂ (20 ml), washed with water (2×10 ml) and brine (10 ml), and dried over MgSO₄, filtered, and concentrated. The residue was dissolved in CH₂Cl₂ (5 ml) and then a solution of methanesulfonic acid (0.07 g, 0.73 mmol) in ethyl acetate (20 ml) was added. The precipitate was collected by filtration to afford trioxolane OZ491 (0.34 g, 94%) as a colorless solid. mp 142-143° C.; ¹H NMR (500 MHz, DMSO-d₆) δ 1.48-1.59 (m, 2H), 1.62-2.08 (m, 22H), 2.32 (s, 3H), 2.86-2.96 (m, 1H), 2.97-3.06 (m, 2H), 4.04 (t, J=5.8 Hz, 2H), 6.88-6.96 (m, 2H), 7.09-7.19 (m, 2H), 7.73 (brs, 3H); ¹³C NMR (125.7 MHz, DMSO-d₆) δ 25.98, 26.41, 27.28, 29.77, 34.43, 34.62, 35.28, 35.99, 36.25, 36.71, 64.63, 108.40, 110.70, 111.82, 120.91, 126.15, 127.19, 133.76, 155.60. Anal. Calcd for C₂₆H₃₉NO₇S: C, 61.27; H, 7.71; N, 2.75. Found: C, 60.98; H, 7.82; N, 2.65.

Adamantane-2-spiro-3′-1′,2′,4′-trioxaspiro[4.6]undecane (OZ492). A solution of O-methyl 2-admantanone oxime (1.35 g, 7.5 mmol) and cycloheptanone (0.57 g, 5.0 mmol) in DCM (10 ml) and pentane (50 ml) was treated with ozone according to the general procedure. The reaction solution was then concentrated to dryness. The residue was purified by flash chromatography (silica gel, 1% ethyl ether in hexane) twice to afford trioxolane OZ492 (250 mg, 18%) as a colorless oil. ¹H NMR (500 MHz, CDCl₃) δ 1.49-2.11 (m, 26H); ¹³C NMR (125.7 MHz, CDCl₃) δ 22.68, 26.51, 26.94, 29.33, 34.77, 34.84, 36.25, 36.85, 37.65, 111.44, 113.08. Anal. Calcd for C₁₇H₂₆O₃: C, 73.34; H, 9.41. Found: C, 73.50; H, 9.61.

cis-Adamantane-2-spiro-3′-8′-[4′-[3′-(methylamino)propoxy]phenyl]-1′,2′,4′-trioxaspiro[4.5]decane mesylate (OZ494). To a solution of OZ288 (0.50 g, 1.4 mmol) in dry acetonitrile (50 ml) were added powered NaOH (0.34 g, 8.5 mmol) and tetrabutylammonium hydrogensulfate (0.05 g, 0.15 mmol). The mixture was stirred at rt for 30 min before N-methyl-3-chloropropylamine hydrochloride (0.60 g, 4.17 mmol) was added. The reaction mixture was stirred at 60° C. overnight, cooled to rt, filtered, and washed with CH₂Cl₂. After the filtrate was concentrated, the residue was dissolved in CH₂Cl₂ (20 ml), washed with water (2×10 ml) and brine (10 ml), and dried over MgSO₄, filtered, and concentrated. The residue was dissolved in CH₂Cl₂ (5 ml) and then a solution of methanesulfonic acid (0.10 g, 1.04 mmol) in ethyl acetate (20 ml) was added. The precipitate was collected by filtration to afford trioxolane OZ494 (0.42 g, 57%) as a colorless solid. mp 125-126° C.; ¹H NMR (500 MHz, DMSO-d₆) δ 1.48-1.59 (m, 2H), 1.62-2.08 (m, 22H), 2.33 (s, 3H), 2.51-2.60 (m, 1H), 2.59 (t, J=5.3 Hz, 3H), 3.01-3.09 (m, 2H), 4.01 (t, J=5.9 Hz, 2H), 6.86 (d, J=8.8 Hz, 2H), 7.13 (d, =8.3 Hz, 2H), 8.34 (brs, 2H); ¹³C NMR (125.7 MHz, DMSO-d₆) δ 25.62, 25.98, 26.39, 31.49, 32.87, 34.26, 34.43, 35.96, 36.25, 40.86, 46.09, 64.70, 108.31, 110.72, 114.57, 127.65, 138.42, 156.69. Anal. Calcd for C₂₇H₄₁NO₇S.1H₂O: C, 59.87; H, 8.00; N, 2.59. Found: C, 59.74; H, 7.71; N, 2.60.

cis-Adamantane-2-spiro-3′-8′-[4′-[2′-(2′-aminoethoxy)ethoxy]phenyl]-1′,2′,4′-trioxaspiro[4.5]decane mesylate (OZ496). Step 1. A mixture of phthalic anhydride (14.8 g, 0.10 mol) and 2-(2-aminoethoxy)ethanol (10.5 g, 0.10 mol) in toluene (250 ml) was heated under reflux for 4 h. After the solvent was removed under vacuum, the residue solidified upon cooling. Crystallization from chloroform gave N-[2-(2-hydroxyethoxy)ethyl]phthalimide (20.0 g, 85%) as a pale yellow solid. mp 63-64° C.; ¹H NMR (500 MHz, CDCl₃) δ 3.54-3.82 (m, 6H), 3.85-3.98 (m, 2H), 7.66-7.78 (m, 2H), 7.80-7.90 (m, 2H). Step 2. Diisopropyl azodicarboxylate (1.4 ml, 7.02 mmol) was added dropwise to a mixture of OZ288 (1.00 g, 2.81 mmol), N-[2-(2-hydroxyethoxy)ethyl]phthalimide (1.3 g, 5.53 mmol), triphenylphosphine (1.84 g, 7.02 mmol), and triethylamine (1.0 ml, 7.02 mmol) in THF (50 ml) at 0° C. under N₂ atmosphere. The resulting mixture was stirred at rt for 24 h. After removal of the solvent, the crude product was purified by crystallization from EtOH to afford the desired phenol ether (1.10 g, 68%) as a colorless solid. mp 140-141° C.; ¹H NMR (500 MHz, CDCl₃) δ 1.64-2.10 (m, 22H), 2.44-2.54 (m, 1H), 3.81-3.90 (m, 4H), 3.91-3.98 (m, 2H), 4.03-4.10 (m, 2H), 6.78 (d, J=9.0 Hz, 2H), 7.08 (d, J=9.0 Hz, 2H), 7.68-7.78 (m, 2H), 7.80-7.90 (m, 2H); ¹³C NMR (125.7 MHz, CDCl₃) δ 26.49, 26.89, 31.63, 34.75, 34.81, 36.41, 36.81, 37.29, 42.03, 67.47, 68.07, 69.18, 108.44, 111.35, 114.47, 123.24, 127.51, 132.11, 133.87, 138.48, 157.03, 168.29. Step 3. A mixture of the above phenol ether (1.00 g, 1.75 mmol) and hydrazine monohydrate (2 ml) in chloroform (80 ml) and methanol (9 ml) was heated at 50° C. for 24 h. After the reaction mixture was cooled to rt and filtered to remove the solid by-product, the filtrate was washed with water (2×40 ml) and brine (40 ml), dried over MgSO₄, filtered, and concentrated. The residue was dissolved in CH₂Cl₂ (5 ml) and a solution of methanesulfonic acid (0.17 g, 1.8 mmol) in ethyl acetate (20 ml) was added. The precipitate was collected by filtration to afford trioxolane OZ496 (0.85 g, 90%) as a colorless solid. mp 148-149° C.; ¹H NMR (500 MHz, DMSO-d₆) δ 1.48-1.59 (m, 2H), 1.62-1.97 (m, 20H), 2.30 (brs, 3H), 2.51-2.60 (m, 1H), 3.00 (t, J=5.1 Hz, 2H), 3.66 (t, J=5.1 Hz, 2H), 3.78 (t, J=4.4 Hz, 2H), 4.09 (t, J=4.4 Hz, 2H), 6.86 (d, J=8.3 Hz, 2H), 7.13 (d, =8.8 Hz, 2H), 7.76 (brs, 3H); ¹³C NMR (125.7 MHz, DMSO-d₆) δ 26.21, 26.61, 31.70, 34.50, 34.66, 36.19, 36.46, 38.85, 41.05, 66.98, 67.29, 69.39, 108.59, 111.03, 114.75, 127.92, 138.58, 157.03. Anal. Calcd for C₂₇H₄₁NO₈S: C, 60.09; H, 7.66; N, 2.60. Found: C, 60.03; H, 7.52; N, 2.63.

cis-Adamantane-2-spiro-3′-8′-[4′-[2′-(tert-butylamino)ethoxy]phenyl]-1′,2′,4′-trioxaspiro[4.5]decane mesylate (OZ497). Step 1. A two-phase mixture of 4-(4-hydroxyphenyl)cyclohexanone (10 g, 53 mmol), 1,2-dibromoethane (30 ml), tetrabutylammonium hydrogen sulfate (0.3 g, 1 mmol), and 3 M aq. NaOH (25 ml) was refluxed overnight. After the reaction mixture was cooled to rt and filtered to remove the solid unreacted phenol, the filtrate was washed with water (2×5 ml) and brine (5 ml), dried over MgSO₄, filtered, and concentrated. The residue was purified by crystallization from MeOH/H₂O (10:1) to afford 4-[4-(2-bromoethoxy)phenyl]cyclohexanone (6.5 g, 42%) as a colorless solid. mp 91-92° C.; ¹H NMR (500 MHz, CDCl₃) δ 1.84-1.98 (m, 2H), 2.15-2.24 (m, 2H), 2.44-2.54 (m, 4H), 2.94-3.04 (m, 1H), 3.63 (t, J=6.5 Hz, 2H), 4.28 (t, J=6.5 Hz, 2H), 6.88 (d, J=9.0 Hz, 2H), 7.17 (d, J=9.0 Hz, 2H); ¹³C NMR (125.7 MHz, CDCl₃) δ 29.12, 34.15, 41.35, 41.92, 67.96, 114.89, 127.69, 137.76, 156.72, 211.15. Step 2. A solution of O-methyl 2-adamantanone oxime (0.58 g, 3.2 mmol) and 4-[4-(2-bromoethoxy)phenyl]cycloheanone (0.63 g, 2.0 mmol) in cyclohexane (60 ml) and CH₂Cl₂ (20 ml) was treated with ozone according to the general procedure. After removal of the solvents, the crude product was purified by crystallization from EtOH/H₂O (10:2) to afford cis-adamantane-2-spiro-3′-8′-[4′-(2′-bromoethoxy)phenyl]-1′,2′,4′-trioxaspiro[4.5]decane (0.41 g, 42%) as a colorless solid. mp 85-86° C.; ¹H NMR (500 MHz, CDCl₃) δ 1.60-2.10 (m, 22H), 2.45-2.55 (m, 1H), 3.62 (t, J=6.5 Hz, 2H), 4.26 (t, J=6.5 Hz, 2H), 6.84 (d, J=8.0 Hz, 2H), 7.12 (d, J=8.0 Hz, 2H); ¹³C NMR (125.7 MHz, CDCl₃) δ 26.49, 26.89, 29.19, 31.62, 34.73, 34.81, 36.41, 36.81, 42.08, 67.95, 108.40, 111.39, 114.72, 127.76, 139.22, 156.43. Step 3. A mixture of cis-adamantane-2-spiro-3′-8′-[4′-(2′-bromoethoxy)phenyl]-1′,2′,4′-trioxaspiro[4.5]decane (0.15 g, 0.32 mmol), tert-butylamine (2 ml), and K₂CO₃ (1.0 g) in dry acetonitrile (20 ml) was heated at 60° C. for 24 h. After the reaction mixture was cooled to rt and filtered to remove the solid material, the filtrate was concentrated. The residue was dissolved in CH₂Cl₂ (5 ml) and a solution of methanesulfonic acid (0.03 g, 0.31 mmol) in ethyl acetate (20 ml) was added. The precipitate was collected by filtration to afford trioxolane OZ497 (0.17 g, 94%) as a colorless solid. mp 156-157° C.; ¹H NMR (500 MHz, CDCl₃) δ 1.44 (s, 9H), 1.61-2.08 (m, 22H), 2.41-2.53 (m, 1H), 2.60 (s, 3H), 3.28 (brs, 2H), 4.35 (t, J=5.9 Hz, 2H), 6.85 (d, J=8.3 Hz, 2H), 7.07 (d, J=8.3 Hz, 2H), 8.56 (brs, 2H); ¹³C NMR (125.7 MHz, CDCl₃) δ 25.70, 26.50, 26.89, 31.60, 34.71, 34.81, 36.41, 36.82, 39.44, 41.09, 42.00, 57.40, 62.94, 108.38, 111.36, 114.61, 127.68, 139.17, 156.11. Anal. Calcd for C₂₉H₄₅NO₇S: C, 63.13; H, 8.22; N, 2.54. Found: C, 63.12; H, 7.96; N, 2.46.

Adamantane-2-spiro-3′-1′,2′,4′-trioxaspiro[4.4]nonane (OZ500). A solution of O-methyl 2-admantanone oxime (1.35 g, 7.5 mmol and cyclopentanone (0.43 g, 5.0 mmol) in DCM (10 ml) and pentane (50 ml) was treated with ozone according to the general procedure. The reaction solution was then concentrated to dryness. The residue was purified by flash chromatography (silica gel, 1% ethyl ether in hexane) twice to afford trioxolane OZ500 (210 mg, 17%) as a colorless oil. ¹H NMR (500 MHz, CDCl₃) δ 1.49-2.12 (m, 22H); ¹³C NMR (125.7 MHz, CDCl₃) δ 23.69, 26.48, 26.88, 34.70, 34.85, 35.28, 35.99, 36.81, 111.15, 118.14. Anal. Calcd for C₁₅H₂₂O₃: C, 71.97; H, 8.86. Found: C, 71.79; H, 8.98.

cis-Adamantane-2-spiro-3′-8′-[4′-[2′-[(2′-hydroxy-2′-methylpropyl)amino]ethoxy]phenyl]-1′,2′,4′-trioxaspiro[4.5]decane p-tosylate (OZ501). A solution of 1,2-epoxy-2-methylpropane (1 ml, 11 mmol) and the free base of OZ323 (600 mg, 1.5 mmol) in ethanol (10 ml) under Ar was stirred at rt for 2 d and then evaporated to dryness. The residue was dissolved in DCM (30 ml), washed with water (5×30 ml), dried over MgSO₄, and evaporated to dryness. The crude product was dissolved in DCM (10 ml), cooled in ice-water bath, and treated with a solution of p-toluenesulfonic acid monohydrate (270 mg, 1.4 mmol) in ethyl ether (30 ml). The resulting precipitate was collected through filtration, washed with ether, and air dried to give trioxolane OZ501 (710 mg, 74%) as a yellowish solid. mp 145-147° C.; ¹H NMR (500 MHz, DMSO-d₆) δ 1.21 (s, 6H), 1.45-1.61 (m, 2H), 1.62-1.98 (m, 20H), 2.29 (s, 3H), 2.50-2.63 (m, 1H), 2.95-3.05 (m, 2H), 3.31-3.42 (m, 3H), 4.24 (t, J=5.1 Hz, 2H), 6.90 (d, J=8.3 Hz, 2H), 7.11 (d, J=7.8 Hz, 2H), 7.16 (d, J=8.3 Hz, 2H), 7.49 (d, J=8.3 Hz, 2H), 8.39 (brs, 2H); ¹³C NMR (125.7 MHz, DMSO-d₆) 20.95, 25.99, 26.40, 27.53, 31.47, 34.26, 34.44, 35.97, 36.26, 40.88, 47.11, 57.25, 63.08, 67.34, 108.30, 110.73, 114.73, 125.67, 127.73, 128.23, 137.82, 138.91, 145.83, 156.20. Anal. Calcd for C₃₅H₄₉NO₈S: C, 65.29; H, 7.67; N, 2.18. Found: C, 65.14; H, 7.57; N, 1.99.

cis-Adamantane-2-spiro-3′-8′-[4′-[3′-(4′-trifluoromethyl-1′-piperidinyl)propoxy]phenyl]-1′,2′,4′-trioxaspiro[4.5]decane mesylate (OZ502). Step 1. To a mixture of 4-(trifluoromethyl)piperidine hydrochloride (1.00 g, 5.29 mmol) and potassium carbonate (3.70 g, 26.45 mmol) in acetonitrile (50 ml) at rt was added 3-bromopropanol (1.10 g, 7.93 mmol). The reaction mixture was refluxed for 5 h and cooled to rt. After the reaction mixture was filtered, the filtrate was concentrated under vacuum. The residue was dissolved in 1,2-dichloroethane (50 ml) and thionyl chloride (5 ml) was added at rt. The reaction mixture was refluxed overnight and then cooled to rt. After the solvent was removed under vacuum, the residue was triturated with ether (50 ml). The resulting precipitate was filtered, washed with ether (3×25 ml), and dried at 50° C. to afford 1-(3-chloropropyl)-4-(trifluoromethyl)piperidine hydrochloride (1.40 g, 100%) as a colorless solid. ¹H NMR (500 MHz, CDCl₃) δ 2.06-2.16 (m, 2H), 2.34-2.52 (m, 5H), 2.81-2.92 (m, 2H), 3.16-3.24 (m, 2H), 3.68 (t, J=5.9 Hz, 4H), 12.08 (brs, 1H). Step 2. To a solution of OZ288 (0.50 g, 1.4 mmol) in dry acetonitrile (50 ml) were added powdered NaOH (0.23 g, 5.62 mmol) and tetrabutylammonium hydrogensulfate (0.10 g, 0.28 mmol). After the reaction mixture was stirred at rt for 30 min, 1-(3-chloropropyl)-4-(trifluoromethyl)piperidine hydrochloride (0.75 g, 2.8 mmol) was added. The reaction mixture was stirred at 60° C. overnight and cooled to rt. The inorganic solid was filtered off and washed with EtOAc (2×25 ml). After removal of the solvent under vacuum, the residue was dissolved in EtOAc (50 ml). The organic layer was washed with water and brine and dried over MgSO₄. After the solvent was removed under vacuum, the residue was purified by chromatography (silica gel, 50% EtOAc in hexane) to afford the desired free base (0.69 g, 90%) as a colorless solid. ¹H NMR (500 MHz, CDCl₃) δ 1.58-2.04 (m, 311H), 2.46-2.51 (m, 3H), 3.00-3.02 (m, 2H), 3.98 (t, J=6.4 Hz, 2H), 6.82 (d, J=8.8 Hz, 2H), 7.10 (d, J=8.3 Hz, 2H); ¹³C NMR (125.7 MHz, CDCl₃) δ 24.66 (q, J=5.0 Hz), 26.47, 26.87, 26.94, 31.64, 40.41 (q, J=27.0 Hz), 42.03, 52.51, 55.14, 66.11, 108.41, 111.32, 114.35, 127.49 (q, J=278.5 Hz), 127.56, 138.28, 157.32. To a solution of the above free base (0.69 g, 1.26 mmol) in EtOAc (10 ml) at 0° C. was added dropwise a solution of methanesulfonic acid (0.14 g, 1.4 mmol) in ether (10 ml). The resulting precipitate was filtered, washed with ether (3×10 ml), and dried under vacuum at 40° C. to afford trioxolane OZ502 (0.72 g, 89%) as a colorless solid. mp 152-154° C.; ¹H NMR (500 MHz, CDCl₃) δ 1.59-2.42 (m, 29H), 2.44-2.57 (m, 1H), 2.78 (s, 3H), 2.71-2.85 (m, 2H), 3.21-3.32 (m, 2H), 3.77 (d, J=11.2 Hz, 2H), 4.06 (t, J=5.6 Hz, 2H), 6.78 (d, J=8.3 Hz, 2H), 7.11 (d, J=8.3 Hz, 2H), 11.06 (brs, 1H); ¹³C NMR (125.7 MHz, CDCl₃) δ 22.11, 24.08, 26.44, 26.84, 31.57, 34.66, 34.76, 36.37, 36.75, 38.38 (q, J=28.9 Hz), 39.38, 41.95, 48.80, 51.64, 55.55, 64.66, 108.33, 111.36, 114.23, 126.04 (q, J=278.0 Hz), 127.74, 139.12, 156.44. Anal. Calcd for C₃₂H₄₆F₃NO₇S: C, 59.52; H, 7.18; N, 2.17. Found: C, 59.53; H, 6.97; N, 2.05.

cis-Adamantane-2-spiro-3′-8′-[4′-[2′-(1′H-imidazol-1′-yl)ethoxy]phenyl]-1′,2′,4′-trioxaspiro[4.5]decane mesylate (OZ503). To a solution of OZ288 (0.60 g, 1.69 mmol), N-(2-hydroxyethyl)imidazole (0.28 g, 2.53 mmol), triphenylphosphine (0.66 g, 2.53 mmol), and triethylamine (0.35 ml, 2.53 mmol) in dry THF (50 ml) at 0° C. was added dropwise a solution of DIAD (0.51 g, 2.53 mmol) in THF (10 ml). After the reaction mixture was stirred at rt overnight, the solvent was removed under vacuum. The residue was dissolved in EtOAc (50 ml), washed with water (3×50 ml), dried over MgSO₄, and concentrated. The residue was purified by chromatography (silica gel, 50% EtOH in EtOAc) to afford the desired free base (0.51 g, 67%) as a colorless solid. To a solution of the above free base (0.51 g, 1.13 mmol) in EtOAc (10 ml) at 0° C. was added dropwise a solution of methanesulfonic acid (0.11 g, 1.13 mmol) in ether (10 ml). The resulting precipitate was filtered, washed with ether (25 ml), and dried under vacuum at 40° C. to afford trioxolane OZ503 (0.58 g, 93%) as a colorless solid. mp 140-142° C.; ¹H NMR (500 MHz, CDCl₃) δ 1.59-2.08 (m, 22H), 2.42-2.55 (m, 1H), 2.83 (s, 3H), 4.31 (t, J=4.7 Hz, 2H), 4.73 (t, J=4.4 Hz, 2H), 6.80 (d, J=8.3 Hz, 2H), 7.11 (d, J=8.8 Hz, 2H), 7.36 (s, 1H), 7.39 (s, 1H), 9.33 (s, 1H); ¹³C NMR (125.7 MHz, CDCl₃) δ 26.43, 26.82, 31.53, 34.62, 34.75, 36.35, 36.74, 39.62, 41.95, 48.90, 66.52, 108.28, 111.35, 114.35, 119.94, 121.86, 127.84, 136.21, 139.68, 155.77. Anal. Calcd for C₂₈H₃₈N₂O₇S: C, 61.52; H, 7.01; N, 5.12. Found: C, 61.44; H, 6.91; N, 5.01.

cis-Adamantane-2-spiro-3′-8′-[4′-[3′-(4′,4′-difluoro-1′-piperidinyl)propoxy]phenyl]-1′,2′,4′-trioxaspiro[4.5]decane mesylate (OZ504). Step 1. To a mixture of 4,4-difluoropiperidine hydrochloride (1.0 g, 6.37 mmol) and potassium carbonate (4.40 g, 31.48 mmol) in acetonitrile (50 ml) at rt was added 3-bromopropanol (1.33 g, 9.55 mmol). The reaction mixture was refluxed for 5 h and cooled to rt. After the reaction mixture was filtered, the filtrate was concentrated under vacuum. The residue was dissolved in 1,2-dichloroethane (50 ml) and thionyl chloride (5 ml) was added at rt. The reaction mixture was refluxed overnight and cooled to rt. After the solvent was removed under vacuum, the residue was triturated with ether (50 ml). The resulting precipitate was filtered, washed with ether (3×25 ml), and dried at 50° C. to afford 1-(3-chloropropyl)-4,4-difluoropiperidine hydrochloride (1.30 g, 88%) as a colorless solid. ¹H NMR (500 MHz, DMSO-d₆) δ 2.21-2.32 (m, 4H), 2.46-2.58 (m, 2H), 3.10-3.24 (m, 4H), 3.59-3.62 (m, 2H), 3.76 (t, J=6.4 Hz, 2H), 11.67 (brs, 1H). Step 2. To a solution of OZ288 (0.50 g, 1.4 mmol) in dry acetonitrile (50 ml) were added powdered NaOH (0.23 g, 5.62 mmol) and tetrabutylammonium hydrogensulfate (0.1 g, 0.28 mmol). After the reaction mixture was stirred at rt for 30 min, 1-(3-chloropropyl)-4,4-difluoropiperidine hydrochloride (0.66 g, 2.8 mmol) was added. The reaction mixture was stirred at 60° C. overnight and cooled to rt. The inorganic solid was filtered off and washed with EtOAc (2×25 ml). After removal of the solvent under vacuum, the residue was dissolved in EtOAc (50 ml). The organic layer was washed with water and brine and dried over MgSO₄. After the solvent was removed under vacuum, the residue was purified by chromatography (silica gel, 50% EtOAc in hexane) to afford the desired free base (0.54 g, 74%) as a colorless solid. ¹H NMR (500 MHz, CDCl₃) δ 1.69-2.04 (m, 28H), 2.47-2.51 (m, 1H), 2.54-2.57 (m, 6H), 3.98 (d, J=6.4 Hz, 2H), 6.82 (d, J=8.8 Hz, 2H), 7.10 (d, J=8.8 Hz, 2H); ¹³C NMR (125.7 MHz, CDCl₃) δ 26.47, 26.87, 27.16, 31.64, 33.98 (t, J=22.9 Hz), 34.72, 34.78, 36.39, 36.78, 42.02, 50.04 (t, J=5.3 Hz), 54.19, 65.94, 108.4, 111.33, 114.33, 122.05 (t, J=241.4 Hz), 127.57, 138.32, 157.29. To a solution of the above free base (0.51 g, 0.99 mmol) in EtOAc (10 ml) at 0° C. was added dropwise a solution of methanesulfonic acid (0.10 g, 0.99 mmol) in ether (10 ml). The resulting precipitate was filtered, washed with ether (25 ml), and dried under vacuum at 40° C. to afford trioxolane OZ504 (0.55 g, 91%) as a colorless solid. mp 152-154° C.; ¹H NMR (500 MHz, CDCl₃) δ 1.59-2.08 (m, 22H), 2.21-2.42 (m, 4H), 2.44-2.55 (m, 1H), 2.62-2.81 (m, 2H), 2.78 (s, 3H), 2.97-3.12 (m, 2H), 3.25-3.38 (m, 2H), 3.73 (d, J=11.7 Hz, 2H), 4.07 (t, J=5.0 Hz, 2H), 6.79 (d, J=8.8 Hz, 2H), 7.11 (d, J=8.8 Hz, 2H), 11.43 (brs, 1H); ¹³C NMR (125.7 MHz, CDCl₃) δ 24.32, 26.41, 26.81, 30.99 (t, J=25.9 Hz), 31.54, 34.63, 34.73, 36.34, 36.72, 39.28, 41.92, 49.82, 49.91, 54.96, 64.48, 108.29, 111.33, 114.19, 118.63 (t, J=239.1 Hz), 127.72, 139.15, 156.35. Anal. Calcd for C₃₁H₄₅F₂NO₇S: C, 60.66; H, 7.39; N, 2.28. Found: C, 60.51; H, 7.20; N, 2.26.

cis-Adamantane-2-spiro-3′-8′-[4′-[2′-[(2′-aminoethyl)thio]ethoxy]phenyl]-1′,2′,4′-trioxaspiro[4.5]decane mesylate (OZ506). Step 1. A mixture of phthalic anhydride (6.11 g, 41 mmol) and 2-(2-aminoethylthio)ethanol (5.00 g, 41 mmol) in toluene (100 ml) was heated under reflux overnight. After the solvent was removed under vacuum, the residue was purified by chromatography to afford N-[2-(2-hydroxyethylthio)ethyl]phthalimide (10.0 g, 96%) as a pale yellow oil. ¹H NMR (500 MHz, CDCl₃) δ 2.82 (t, J=6.0 Hz, 2H), 2.86 (t, J=7.0 Hz, 2H), 3.78 (t, J=6.0 Hz, 2H), 3.93 (t, J=7.0 Hz, 2H), 7.68-7.78 (m, 2H), 7.82-7.92 (m, 2H). Step 2. Diisopropyl azodicarboxylate (1.4 ml, 7.02 mmol) was added dropwise to a mixture of OZ288 (1.00 g, 2.81 mmol), N-[2-(2-hydroxyethylthio)ethyl]phthalimide (1.40 g, 5.58 mmol), triphenylphosphine (1.84 g, 7.02 mmol), and triethylamine (1.0 ml, 7.02 mmol) in THF (50 ml) at 0° C. under N₂ atmosphere. The resulting mixture was stirred at rt for 24 h. After removal of the solvent, the crude product was purified by crystallization from EtOH to afford the desired phenol ether (0.8 g, 48%) as a colorless solid. mp 100-102° C.; ¹H NMR (500 MHz, CDCl₃) δ 1.50-2.08 (m, 22H), 2.29 (s, 3H), 2.44-2.54 (m, 1H), 2.91-3.00 (m, 2H), 3.68-3.77 (m, 2H), 3.94 (t, J=7.0 Hz, 2H), 4.14 (t, J=6.5 Hz, 2H), 6.83 (d, J=8.5 Hz, 2H), 7.11 (d, J=8.5 Hz, 2H), 7.69-7.76 (m, 2H), 7.82-7.89 (m, 2H); ¹³C NMR (125.7 MHz, CDCl₃) δ 26.49, 26.89, 30.54, 30.66, 31.64, 34.75, 34.81, 36.41, 36.81, 37.09, 42.07, 67.84, 108.44, 111.36, 114.50, 123.37, 127.67, 132.02, 134.02, 138.73, 156.79, 168.15. Step 3. A mixture of the above phenol ether (0.20 g, 0.30 mmol) and hydrazine monohydrate (1 ml) in chloroform (20 ml) and methanol (3 ml) was heated at 50° C. for 24 h. After the reaction mixture was cooled to rt and filtered to remove the solid by-product, the filtrate was washed with water (2×10 ml) and brine (10 ml), dried over MgSO₄, filtered, and concentrated. The residue was dissolved in CH₂Cl₂ (5 ml) and a solution of methanesulfonic acid (0.03 g, 0.31 mmol) in ethyl acetate (20 ml) was added. The precipitate was collected by filtration to afford trioxolane OZ506 (0.12 g, 63%) as a colorless solid. mp 129-130° C.; ¹H NMR (500 MHz, DMSO-d₆) δ 1.45-1.59 (m, 2H), 1.62-1.98 (m, 20H), 2.29 (s, 3H), 2.50-2.61 (m, 1H), 2.79 (t, J=7.3 Hz, 2H), 2.91 (t, J=6.4 Hz, 2H), 2.98-3.09 (m, 2H), 4.10 (t, J=6.2 Hz, 2H), 6.86 (d, J=8.3 Hz, 2H), 7.12 (d, J=8.3 Hz, 2H), 7.75 (brs, 3H); ¹³C NMR (125.7 MHz, DMSO-d₆) δ 25.98, 26.39, 28.94, 30.28, 31.48, 34.26, 34.43, 35.95, 36.25, 38.78, 39.94, 40.86, 67.49, 108.30, 110.72, 114.56, 127.67, 138.44, 156.64. Anal. Calcd for C₂₇H₄₁NO₇S₂: C, 58.35; H, 7.44; N, 2.52. Found: C, 50.55; H, 7.02; N, 5.45.

cis-Adamantane-2-spiro-3′-8′-[4′-[2′-[(2′-aminoethyl)sulfonyl]ethoxy]phenyl]-1′,2′,4′-trioxaspiro[4.5]decane mesylate (OZ507). Step 1. To a solution of cis-adamantane-2-spiro-3′-8′-[4′-[2′-[(2′-phthalimidoethyl)thio]ethoxy]phenyl-1′,2′,4′-trioxaspiro[4.5]decane (0.40 g, 0.68 mmol) in CH₂Cl₂ (20 ml) at 0° C. was added dropwise a solution of m-CPBA (0.26 g, 1.51 mmol) in CH₂Cl₂ (10 ml). The resulting mixture was stirred at rt for 24 h and then partitioned between CH₂Cl₂ (20 ml) and saturated aq. NaHCO₃ (20 ml). The organic layer was washed with water (20 ml) and brine (20 ml), dried over MgSO₄, and filtered. After removal of the solvent, the crude product was purified by crystallization from CH₂Cl₂/EtOH (1:10) to afford the desired sulfone (0.40 g, 95%) as a colorless solid. mp 144-145° C.; ¹H NMR (500 MHz, CDCl₃) δ 1.64-2.08 (m, 22H), 2.45-2.55 (m, 1H), 3.52-3.60 (m, 4H), 4.27 (t, J=6.0 Hz, 2H), 4.40 (t, J=5.0 Hz, 2H), 6.83 (d, J=8.5 Hz, 2H), 7.13 (d, J=8.5 Hz, 2H), 7.70-7.77 (m, 2H), 7.84-7.91 (m, 2H); ¹³C NMR (125.7 MHz, CDCl₃) δ 26.49, 26.89, 31.59, 31.68, 34.70, 34.81, 36.41, 36.80, 42.07, 52.10, 52.68, 62.13, 108.35, 111.42, 114.48, 123.60, 127.94, 131.90, 134.23, 139.88, 155.81, 167.78. Step 2. A mixture of the above sulfone (0.40 g, 0.60 mmol) and hydrazine monohydrate (1 ml) in chloroform (40 ml) and methanol (6 ml) was heated at 50° C. for 24 h. After the reaction mixture was cooled to rt and filtered to remove the solid by-product, the filtrate was washed with water (2×10 ml) and brine (10 ml), dried over MgSO₄, filtered, and concentrated. The residue was dissolved in CH₂Cl₂ (5 ml) and then a solution of methanesulfonic acid (0.05 g, 0.52 mmol) in ethyl acetate (20 ml) was added. The precipitate was collected by filtration to afford trioxolane OZ507 (0.21 g, 55%) as a colorless solid. mp 145-146° C.; ¹H NMR (500 MHz, DMSO-d₆) δ 1.45-1.59 (m, 2H), 1.62-1.98 (m, 20H), 2.29 (s, 3H), 2.50-2.61 (m, 1H), 3.21-3.31 (m, 2H), 3.52 (t, J=7.6 Hz, 2H), 3.76 (t, J=5.2 Hz, 2H), 4.30 (t, J=5.4 Hz, 2H), 6.92 (d, J=8.8 Hz, 2H), 7.15 (d, J=8.8 Hz, 2H), 7.90 (brs, 3H); ¹³C NMR (125.7 MHz, DMSO-d₆) δ 25.97, 26.39, 31.46, 32.66, 34.26, 34.43, 35.96, 36.25, 51.54, 61.95, 108.30, 110.74, 114.66, 127.74, 139.09, 156.04. Anal. Calcd for C₂₇H₄₁NO₉S₂: C, 55.18; H, 7.03; N, 2.38. Found: C, 55.10; H, 6.91; N, 2.35.

cis-Adamantane-2-spiro-3′-8′-(4′-aminocyclohexyl)-1′,2′,4′-trioxaspiro[4.5]decane mesylate (OZ509). Step 1. Diisopropyl azodicarboxylate (1.22 ml, 6.1 mmol) was added dropwise to a mixture of OZ508 (2.2 g, 6.1 mmol), phthalimide (0.90 g, 6.1 mmol), and triphenylphosphine (1.60 g, 6.1 mmol) in THF (100 ml) at 0° C. under N₂ atmosphere. The resulting mixture was stirred at rt for 24 h. After removal of the solvent, the crude product was purified by crystallization from EtOH to afford the desired phthalimido derivative. ¹H NMR (500 MHz, CDCl₃) δ 1.07-2.38 (m, 32H), 4.10-4.30 (m, 1H), 7.67-7.73 (m, 2H), 7.78-7.84 (m, 2H). Step 2. A mixture of the above phthalimido derivative (2.5 g, 5.1 mmol) and hydrazine monohydrate (3 ml) in chloroform (50 ml) and methanol (10 ml) was heated at 50° C. for 24 h. After the reaction mixture was cooled to rt and filtered to remove the solid by-product, the filtrate was washed with water (2×10 ml) and brine (10 ml), dried over MgSO₄, filtered, and concentrated. The residue was dissolved in CH₂Cl₂ (10 ml) and a solution of methanesulfonic acid (0.48 g, 5.0 mmol) in ethyl acetate (50 ml) was added. The precipitate was collected by filtration to afford trioxolane OZ509 (1.15 g, 98%) as a colorless solid. mp 151-152° C.; ¹H NMR (500 MHz, CDCl₃) δ 1.09-1.29 (m, 4H), 1.47-2.05 (m, 28H), 2.74 (s, 3H), 3.28-3.48 (m, 1H), 7.53 (brs, 3H); ¹³C NMR (125.7 MHz, CDCl₃) δ 23.92, 26.49, 26.88, 27.35, 27.83, 34.34, 34.79, 36.38, 36.81, 39.42, 40.03, 48.18, 108.81, 111.20. Anal. Calcd for C₂₃H₃₉NO₆S.0.5H₂O: C, 59.20; H, 8.64; N, 3.00. Found: C, 58.80; H, 8.40; N, 3.43.

cis-Adamantane-2-spiro-3′-8′-(4′-oxocyclohexyl)-1′,2′,4′-trioxaspiro[4.5]decane oxime (OZ510). To a solution of OZ495 (0.55 g, 1.54 mol) in ethanol (20 ml) was added pyridine (0.20 ml, 2.41 mol) followed by hydroxylamine hydrochloride (0.12 g, 1.73 mol). The reaction mixture was stirred at rt for 48 h, concentrated in vacuo, and diluted with water (20 ml). The solid was collected by filtration to afford trioxolane OZ510 (0.51 g, 89%) as a colorless solid. mp 158-160° C.; ¹H NMR (500 MHz, CDCl₃) δ 1.07-1.42 (m, 6H), 1.58-2.09 (m, 25H), 2.38-2.43 (m, 1H), 3.25-3.36 (m, 1H); ¹³C NMR (125.7 MHz, CDCl₃) δ 23.75, 26.48, 26.88, 27.10, 27.16, 28.75, 29.95, 31.62, 34.45, 34.79, 36.38, 36.80, 40.87, 41.67, 108.83, 111.26, 160.95. Anal. Calcd for C₂₂H₃₃NO₄: C, 70.37; H, 8.86; N, 3.73. Found: C, 70.63; H, 8.69; N, 3.25.

cis-Adamantane-2-spiro-3′-8′-[4′-(4′-piperidinylmethoxy)phenyl]-1′,2′,4′-trioxaspiro[4.5]decane mesylate (OZ511). Step 1. To a stirred solution of 4-piperidinemethanol (2.00 g, 17.4 mmol) in CH₂Cl₂ (25 ml) and 1,4-dioxane (25 ml) was added Boc₂O (3.80 g, 17.4 mmol). The resulting mixture was stirred at rt for 2 h. After evaporation of the solvents, the residue was partitioned between EtOAc (100 ml) and saturated aq. NH₄Cl (50 ml). The organic layer was washed with brine (50 ml), dried over MgSO₄, filtered, and concentrated to give N-Boc-4-piperidinemethanol (2.30 g, 61%) as a colorless solid. mp 80-81° C.; ¹H NMR (500 MHz, CDCl₃) δ 1.09-1.20 (m, 2H), 1.46 (s, 9H), 1.60-1.76 (m, 3H), 2.62-2.78 (m, 2H), 3.46-3.54 (m, 2H), 4.02-4.24 (m, 2H). Step 2. Diisopropyl azodicarboxylate (1.14 ml, 5.7 mmol) was added dropwise to a mixture of OZ288 (2.0 g, 5.6 mmol), N-Boc-4-piperidinemethanol (1.20 g, 5.6 mmol), and triphenylphosphine (1.5 g, 5.7 mmol) in THF (100 ml) at 0° C. under N₂. The resulting mixture was stirred at rt for 24 h. After removal of the solvent, the crude product was purified by crystallization from EtOH to afford the desired phenol ether (2.60 g, 84%) as a colorless solid. mp 146-147° C.; ¹H NMR (500 MHz, CDCl₃) δ 1.20-1.31 (m, 2H), 1.46 (s, 9H), 1.64-2.08 (m, 25H), 2.45-2.54 (m, 1H), 2.67-2.80 (m, 2H), 3.77 (d, J=6.5 Hz, 2H), 4.04-4.24 (m, 2H), 6.81 (d, J=8.5 Hz, 2H), 7.11 (d, J=8.5 Hz, 2H); ¹³C NMR (125.7 MHz, CDCl₃) δ 26.49, 26.89, 28.46, 31.65, 34.74, 34.81, 36.23, 36.41, 36.81, 42.05, 72.33, 79.36, 108.43, 111.37, 114.32, 127.62, 138.40, 154.87, 157.35. Step 3. A mixture of the above phenol ether (2.30 g, 4.16 mmol) and 1.5 M MsOH in THF (27.7 ml) was stirred at rt for 6 h. The resulting precipitate was filtered off, and washed with ether (30 mL), and dried to afford trioxolane OZ511 (2.10 g, 91%) as a colorless solid. mp 95-96° C.; ¹H NMR (500 MHz, DMSO-d₆) δ 1.38-1.59 (m, 4H), 1.61-1.95 (m, 22H), 1.96-2.08 (m, 1H), 2.33 (s, 3H), 2.49-2.60 (m, 1H), 2.82-2.96 (m, 2H), 3.30 (d, J=12.2 Hz, 2H), 3.81 (d, J=6.3 Hz, 2H), 6.84 (d, J=8.8 Hz, 2H), 7.10 (d, J=8.8 Hz, 2H), 8.19 (brs, 1H), 8.49 (brs, 1H); ¹³C NMR (125.7 MHz, DMSO-d₆) δ 25.42, 26.00, 26.41, 31.50, 33.21, 34.28, 34.45, 35.97, 36.27, 40.87, 43.09, 71.25, 108.33, 110.74, 114.53, 127.68, 138.29, 156.98. Anal. Calcd for C₂₉H₄₃NO₇S: C, 63.36; H, 7.88; N, 2.55. Found: C, 60.54; H, 7.48; N, 2.37.

cis-Adamantane-2-spiro-3′-8′-[4′-[2′-[4′-(aminocarbonyl)-1′-piperidinyl]ethoxy]phenyl]-1′,2′,4′-trioxaspiro[4.5]decane (OZ512). A mixture of cis-adamantane-2-spiro-3′-8′-[4′-(2′-bromoethoxy)phenyl]-1′,2′,4′-trioxaspiro[4.5]decane (0.50 g, 1.08 mmol), isonipecotamide (0.35 g, 2.73 mmol), and K₂CO₃ (2.0 g) in dry acetonitrile (80 ml) was heated at 60° C. for 2 d. After the reaction mixture was cooled to rt and filtered to remove the solid material, the filtrate was concentrated. The residue was washed with water (50 ml) and dried in vacuo to afford trioxolane OZ512 (0.35 g, 64%) as a colorless solid. mp 152-153° C.; ¹H NMR (500 MHz, CDCl₃) δ 1.59-2.23 (m, 29H), 2.45-2.57 (m, 1H), 2.79 (t, J=5.9 Hz, 2H), 3.04 (d, J=11.7 Hz, 2H), 4.07 (t, J=5.6 Hz, 2H), 5.29 (brs, 1H), 5.45 (brs, 1H), 6.83 (d, J=8.8 Hz, 2H), 7.11 (d, J=8.3 Hz, 2H); ¹³C NMR (125.7 MHz, CDCl₃) δ 26.49, 26.89, 28.93, 31.64, 34.74, 34.81, 36.41, 36.81, 42.05, 53.53, 57.35, 65.98, 108.43, 111.37, 114.47, 127.62, 138.53, 157.08, 177.06. Anal. Calcd for C₃₀H₄₂N₂O₅: C, 70.56; H, 8.29; N, 5.49. Found: C, 68.22; H, 7.58; N, 4.58.

cis-Adamantane-2-spiro-3′-8′-[(4′-hydroxyphenyl)methyl]-1′,2′,4′-trioxaspiro[4.5]decane (OZ514). Step 1. To a solution of 4-benzyloxybenzyl alcohol (10.7 g, 50 mmol) in DCM (200 ml) at 5° C. was added dropwise a solution of PBr₃ (13.5 g, 50 mmol) in DCM (50 ml). The reaction solution was stirred at rt for 2 d and quenched with water (150 ml). The DCM layer was separated, washed with saturated aq. NaHCO₃ (100 ml) and water (2×100 ml), dried over Na₂SO₄, filtered, and evaporated to dryness. The residue was crystallized from hexane to yield 4-benzyloxybenzyl bromide (11.2 g, 81%) as a white powder. ¹H NMR (500 MHz, CDCl₃) δ 4.49 (s, 2H), 5.06 (s, 2H), 6.93 (d, J=8.3 Hz, 2H), 7.31-7.42 (m, 7H); ¹³C NMR (125.7 MHz, CDCl₃) δ 33.9, 70.0, 115.1, 127.4, 128.0, 128.6, 130.2, 130.4, 136.7, 158.9. Step 2. A mixture of 4-benzyloxybenzyl bromide (11.1 g, 40 mmol) and PPh₃ (10.5 g, 40 mmol) in xylene (150 ml) was refluxed for 24 h. The resulting solution was then cooled and kept at 0° C. for 2 h. The crystalline was collected through filtration, washed with ether (2×300 ml), and dried under vacuum to yield (4-benzyloxybenzyl)triphenylphosphonium bromide (20.5 g, 95%) as a white powder. mp 226-227° C.; ¹H NMR (500 MHz, DMSO-d₆) δ 5.05 (s, 2H), 5.10 (d, J=15.1 Hz, 2H), 6.89 (s, 4H), 7.31-7.41 (m, 5H), 7.64-7.76 (m, 12H), 7.89-7.92 (m, 3H); ¹³C NMR (125.7 MHz, DMSO-d₆) δ 27.6 (d, J=46.1 Hz), 69.4, 115.3 (d, J=3.4 Hz), 118.1 (d, J=84.9 Hz), 119.5 (d, J=8.6 Hz), 127.9, 128.0, 128.6, 130.2 (d, J=12.5 Hz), 132.2 (d, J=5.8 Hz), 134.2 (d, J=9.6 Hz), 135.2 (d, J=2.9 Hz), 136.9, 158.4 (d, J=3.8 Hz). Step 3. To a freshly made sodium dimsyl [a mixture of NaH (2.0 g, 60% oil dispersion, 50 mmol) and DMSO (110 ml) was stirred at 60° C. for 1 h and cooled to rt.] was added (4-benzyloxybenzyl)triphenylphosphonium bromide (20.5 g, 38 mmol). After the mixture was stirred at rt for 10 min, 1,4-cyclohexanedione monoethylene ketal (5.93 g, 38 mmol) was added. The resulting solution was stirred at rt for 22 h before being quenched with ice-water (500 ml). The mixture was extracted with ether (4×400 ml). The ether layers were combined and concentrated. The residue was extracted with hexane (4×400 ml). The hexane layers were combined and concentrated. The residue was purified by flash chromatography (silica gel, 1-6% EtOAc in hexane) to yield 8-[(4-benzyloxyphenyl)methylene]-1,4-dioxaspiro[4.5]decane (5.70 g, 44.7%) as a white solid. mp 56-57° C.; ¹H NMR (500 MHz, CDCl₃) δ 1.67 (t, J=6.3 Hz, 2H), 1.77 (t, J=6.3 Hz, 2H), 2.40 (t, J=6.3 Hz, 2H), 2.51 (t, J=6.3 Hz, 2H), 3.97 (s, 4H), 5.04 (s, 2H), 6.24 (s, 1H), 6.92 (d, J=8.3 Hz, 2H), 7.12 (d, J=8.3 Hz, 2H), 7.29-7.43 (m, 5H); ¹³C NMR (125.7 MHz, CDCl₃) δ 25.7, 34.0, 35.3, 36.1, 64.3, 70.0, 108.7, 114.4, 122.8, 127.4, 127.9, 128.5, 129.9, 130.8, 137.1, 139.1, 157.1. Step 4. A suspension of 8-[(4-benzyloxyphenyl)methylene]-1,4-dioxaspiro[4.5]decane (5.40 g, 16.1 mmol) and 10% Pd—C (500 mg) in ether (30 ml) and MeOH (20 ml) under H₂ was stirred at rt for 24 h. The catalyst was then removed by filtration. The filtrate was concentrated, dissolved in a solution of acetone (20 ml) and 12 M aq. HCl (4 ml), and stirred at rt for 3 h. After the reaction mixture was concentrated, the residue was partitioned between water and EtOAc. The organic layer was washed with water and evaporated to dryness to yield 4-[(4-hydroxyphenyl)methyl]cyclohexanone (3.10 g, 94%) as a semi-solid. ¹H NMR (500 MHz, CDCl₃) δ 1.38-1.46 (m, 2H), 1.93-2.03 (m, 3H) 2.27-2.41 (m, 4H), 2.53 (d, J=7.3 Hz, 2H), 6.10 (s, br, 1H), 6.79 (d, J=6.8 Hz, 2H), 7.01 (d, J=6.8 Hz, 2H); ¹³C NMR (125.7 MHz, CDCl₃) δ 32.3, 38.1, 40.7, 41.1, 115.2, 130.0, 132.0, 154.2, 213.7. Step 5. To a mixture of 4-[(4-hydroxyphenyl)methyl]cyclohexanone (3.10 g, 15.2 mmol) and Et₃N (14 ml) in DCM (50 ml) at 5° C. was added dropwise a solution of acetyl chloride (3.60 g, 45.6 mmol) in DCM (10 ml). The resulting solution was stirred at rt for 20 h and then at 35° C. for 3 h. The solution was poured into ice-water (100 ml), acidified with 6 M aq. HCl to pH=5, and extracted with DCM (5×50 ml). The DCM layers were combined, dried over Na₂SO₄, and evaporated to dryness. The residue was crystallized from MeOH to yield 4-[(4-acetoxyphenyl)methyl]cyclohexanone (2.90 g, 78%) as a white powder. ¹H NMR (500 MHz, CDCl₃) δ 1.39-1.48 (m, 2H), 1.96-2.05 (m, 3H) 2.26-2.38 (m, 4H), 2.29 (s, 3H), 2.60 (d, J=6.8 Hz, 2H), 7.01 (d, J=8.3 Hz, 2H), 7.17 (d, J=8.3 Hz, 2H); ¹³C NMR (125.7 MHz, CDCl₃) δ 21.1, 32.3, 38.0, 40.6, 41.5, 121.3, 129.8, 137.8, 148.9, 169.5, 211.8. Step 6. A solution of O-methyl 2-adamantanone oxime (3.24 g, 18 mmol) and 4-[(4-acetoxyphenyl)methyl]cyclohexanone (2.90 g, 15 mmol) in cyclohexane (80 ml) and CH₂Cl₂ (20 ml) was treated with ozone according to the general procedure. After removal of the solvents, the crude product was purified by chromatography (silica gel, 0-7.5% ether in hexane) followed by crystallization from MeOH to yield cis-adamantane-2-spiro-3′-8′-[(4′-acetoxyphenyl)methyl]-1′,2′,4′-trioxaspiro[4.5]decane (2.40 g, 32%) as a white solid. mp 120-121° C.; ¹H NMR (500 MHz, CDCl₃) δ 1.19-1.27 (m, 2H), 1.48-1.98 (m, 21H), 2.28 (s, 3H), 2.48 (d, J=7.3 Hz, 2H), 6.98 (d, J=8.8 Hz, 2H), 7.12 (d, J=8.8 Hz, 2H); ¹³C NMR (125.7 MHz, CDCl₃) δ 21.1, 26.4, 26.8, 29.8, 34.1, 34.73, 34.74, 36.3, 36.8, 38.1, 42.2, 108.8, 111.2, 121.1, 129.9, 138.4, 148.7, 169.6. Step 7. A solution of cis-adamantane-2-spiro-3′-8′-[(4′-acetoxyphenyl)methyl]-1′,2′,4′-trioxaspiro[4.5]decane (840 mg, 2.04 mmol), THF (15 ml), EtOH (15 ml), and 1 M aq. NaOH (15 ml) was stirred at 50° C. for 16 h. The resulting solution was concentrated, mixed with ice-water (20 ml), acidified with 1 M aq. HCl to pH=4, and extracted with DCM (5×30 ml). The DCM layers were combined, dried over Na₂SO₄, and evaporated to dryness. The residue was crystallized from MeOH to afford trioxolane OZ514 (585 mg, 78%) as a colorless solid. mp 132-133° C.; ¹H NMR (500 MHz, CDCl₃) δ 1.13-1.29 (m, 2H), 1.39-1.55 (m, 1H), 1.56-2.05 (m, 20H), 2.41 (d, J=6.8 Hz, 2H), 5.53 (brs, 1H), 6.75 (d, J=8.3 Hz, 2H), 6.97 (d, J=8.8 Hz, 2H); ¹³C NMR (125.7 MHz, CDCl₃) δ 26.40, 26.79, 29.79, 34.09, 34.73, 36.30, 36.72, 38.21, 41.87, 109.09, 111.30, 115.00, 130.05, 132.86, 153.65. Anal. Calcd for C₂₃H₃₀O₄: C, 74.56; H, 8.16. Found: C, 74.38; H, 7.93.

cis-Adamantane-2-spiro-3′-8′-[[4′-(3′-aminopropoxy)phenyl]methyl]-1′,2′,4′-trioxaspiro[4.5]decane p-tosylate (OZ515). A mixture of OZ514 (670 mg, 1.81 mmol), NaOH (340 mg, 8.5 mmol), and Bu₄NHSO₄ (100 mg, 0.29 mmol) in MeCN (30 ml) was stirred at rt for 30 min before 3-chloropropylamine hydrochloride (550 mg, 4.1 mmol) was added. The resulting solution was stirred at 60° C. for 16 h, then poured into ice-water (75 ml), and extracted with DCM (6×30 ml). The DCM layers were combined, washed with water (2×30 ml), dried over Na₂SO₄, and evaporated to dryness to give the desired free base. A solution of the free base (760 mg, 1.78 mmol) and p-toluenesulfonic acid monohydrate (342 mg, 1.78 mmol) in DCM (10 ml) was stirred at rt overnight. The precipitate was collected by filtration, washed with ether (10 ml), and dried under vacuum to yield trioxolane OZ515 (560 mg, 52%) as a colorless solid. mp 154-155° C.; ¹H NMR (500 MHz, DMSO-d₆) δ 1.01-1.17 (m, 2H), 1.41-2.05 (m, 23H), 2.29 (s, 3H), 2.41 (d, J=6.8 Hz, 2H), 2.89-3.02 (m, 2H), 4.00 (t, J=5.8 Hz, 2H), 6.83 (d, J=8.3 Hz, 2H), 7.06 (d, J=8.3 Hz, 2H), 7.12 (d, J=7.8 Hz, 2H), 7.49 (d, J=7.8 Hz, 2H), 7.72 (s, 3H); ¹³C NMR (125.7 MHz, DMSO-d₆) δ 20.94, 25.96, 26.37, 27.05, 29.53, 33.68, 34.41, 35.91, 36.24, 36.63, 37.53, 41.15, 64.55, 108.81, 110.57, 114.30, 125.65, 128.23, 130.06, 132.77, 137.82, 145.80, 156.61. Anal. Calcd for C₃₃H₄₅NO₇S: C, 66.08; H, 7.56; N, 2.34. Found: C, 65.91; H, 7.44; N, 2.46.

cis-3-Phenyl-7,14,15-trioxadispiro[5.1.5.2]pentadecane (OZ516). A solution of O-methyl cyclohexanone oxime (0.90 g, 7.0 mmol) and 4-phenylcyclohexanone (1.20 g, 6.9 mmol) in cyclohexane (100 ml) and CH₂Cl₂ (50 ml) was treated with ozone according to the general procedure. After removal of the solvents, the crude product was purified by chromatography (silica gel, 10% ether in hexane) to afford trioxolane OZ516 (0.63 g, 32%) as a colorless solid. mp 74-75° C.; ¹H NMR (500 MHz, CDCl₃) δ 1.21-2.01 (m, 16H), 2.06 (d, J=13.2 Hz, 2H), 2.49-2.61 (m, 1H), 7.17-7.35 (m, 5H); ¹³C NMR (125.7 MHz, CDCl₃) δ 23.80, 24.90, 31.41, 34.61, 34.65, 42.92, 108.32, 108.96, 126.18, 126.77, 128.40, 146.10. Anal. Calcd for C₁₈H₂₄O₃: C, 74.97; H, 8.39. Found: C, 74.70; H, 8.29.

cis-Adamantane-2-spiro-3′-8′-[4′-[2′-[bis(2′-hydroxyethyl)amino]ethoxy]phenyl]-1′,2′,4′-trioxaspiro[4.5]decane mesylate (OZ517). A mixture of cis-adamantane-2-spiro-3′-8′-[4′-(2′-bromoethoxy)phenyl]-1′,2′,4′-trioxaspiro[4.5]decane (0.30 g, 0.65 mmol), diethanolamine (0.34 g, 3.24 mmol), and K₂CO₃ (2.0 g) in dry acetonitrile (80 ml) was heated at 60° C. for 2 d. After the reaction mixture was cooled to rt and filtered to remove the solid material, the filtrate was concentrated. The residue was washed with water (50 ml) and dried in vacuo. The crude product was dissolved in CH₂Cl₂ (5 ml) and a solution of methanesulfonic acid (0.04 g, 0.42 mmol) in ethyl acetate (20 ml) was added. The precipitate was collected by filtration to afford trioxolane OZ517 (0.16 g, 42%) as a colorless solid. mp 109-110° C.; ¹H NMR (500 MHz, CDCl₃) δ 1.61-2.09 (m, 22H), 2.45-2.58 (m, 1H), 2.61-2.85 (m, 2H), 2.79 (s, 3H), 3.51-3.61 (m, 4H), 3.76-3.84 (m, 2H), 3.96-4.14 (m, 4H), 4.36-4.44 (m, 2H), 6.83 (d, J=8.3 Hz, 2H), 7.14 (d, J=8.8 Hz, 2H), 9.39 (brs, 1H); ¹³C NMR (125.7 MHz, CDCl₃) δ 26.48, 26.88, 31.58, 34.67, 34.80, 36.41, 36.80, 39.25, 42.02, 53.27, 56.05, 57.05, 62.30, 108.31, 111.43, 114.48, 127.98, 140.09, 155.45. Anal. Calcd for C₂₉H₄₅NO₉S: C, 59.67; H, 7.77; N, 2.40. Found: C, 59.45; H, 7.79; N, 2.48.

cis-Adamantane-2-spiro-3′-8′-[4′-[2′-[(2′-hydroxyethyl)ethylamino]ethoxy]phenyl]-1′,2′,4′-trioxaspiro[4.5]decane mesylate (OZ518). A mixture of cis-adamantane-2-spiro-3′-8′-[4′-(2′-bromoethoxy)phenyl]-1′,2′,4′-trioxaspiro[4.5]decane (0.50 g, 1.08 mmol), 2-ethylaminoethanol (0.50 g, 5.62 mmol), and K₂CO₃ (2.0 g) in dry acetonitrile (80 ml) was heated at 60° C. for 2 d. After the reaction mixture was cooled to rt and filtered to remove the solid material, the filtrate was concentrated. The residue was washed with water (50 ml) and dried in vacuo. The crude product was dissolved in CH₂Cl₂ (5 ml) and then a solution of methanesulfonic acid (0.09 g, 0.94 mmol) in ethyl acetate (20 ml) was added. The precipitate was collected by filtration to afford trioxolane OZ518 (0.42 g, 69%) as a colorless solid. mp 133-134° C.; ¹H NMR (500 MHz, CDCl₃) δ 1.45 (t, J=7.2 Hz, 3H), 1.61-2.09 (m, 22H), 2.45-2.58 (m, 1H), 2.82 (s, 3H), 3.14-3.38 (m, 2H), 3.39-3.49 (m, 3H), 3.54-3.79 (m, 2H), 4.01 (brs, 2H), 4.36-4.48 (m, 2H), 6.83 (d, J=8.8 Hz, 2H), 7.14 (d, J=8.3 Hz, 2H), 10.14 (brs, 1H); ¹³C NMR (125.7 MHz, CDCl₃) δ 8.78, 26.47, 26.87, 31.58, 34.67, 34.80, 36.40, 36.79, 39.22, 42.03, 49.80, 52.22, 56.22, 56.37, 62.56, 108.32, 111.44, 114.40, 127.99, 140.02, 155.47. Anal. Calcd for C₂₉H₄₅NO₈S: C, 61.35; H, 7.99; N, 2.47. Found: C, 61.29; H, 7.76; N, 2.21.

cis-Adamantane-2-spiro-3′-8′-[4′-[2′-[(trans-4′-hydroxycyclohexyl)amino]ethoxy]phenyl]-1′,2′,4′-trioxaspiro[4.5]decane mesylate (OZ519). A mixture of cis-adamantane-2-spiro-3′-8′-[4′-(2′-bromoethoxy)phenyl]-1′,2′,4′-trioxaspiro[4.5]decane (0.50 g, 1.08 mmol), trans-4-aminocyclohexanol (0.62 g, 5.40 mmol), and K₂CO₃ (2.0 g) in dry acetonitrile (80 ml) was heated at 60° C. for 2 d. After the reaction mixture was cooled to rt and filtered to remove the solid material, the filtrate was concentrated. The residue was washed with water (50 ml) and dried in vacuo. The crude product was dissolved in CH₂Cl₂ (5 ml) and then a solution of methanesulfonic acid (0.08 g, 0.83 mmol) in ethyl acetate (20 ml) was added. The precipitate was collected by filtration to afford trioxolane OZ519 (0.42 g, 66%) as a colorless solid. mp 131-132° C.; ¹H NMR (500 MHz, CDCl₃) δ 1.22-1.39 (m, 2H), 1.59-2.09 (m, 27H), 2.23 (d, J=11.7 Hz, 2H), 2.44-2.54 (m, 1H), 2.69 (s, 3H), 3.04-3.16 (m, 1H), 3.38 (brs, 2H), 3.62-3.71 (m, 1H), 4.28 (t, J=5.2 Hz, 2H), 6.84 (d, J=8.8 Hz, 2H), 7.11 (d, J=8.8 Hz, 2H), 8.71 (brs, 2H); ¹³C NMR (125.7 MHz, CDCl₃) δ 26.49, 26.79, 26.89, 31.60, 33.06, 34.68, 34.81, 36.41, 36.81, 41.98, 43.86, 56.24, 63.35, 68.85, 108.35, 111.42, 114.50, 127.81, 139.53, 155.92. Anal. Calcd for C₃₁H₄₇NO₈S.1H₂O: C, 60.86; H, 8.07; N, 2.29. Found: C, 60.30; H, 7.92; N, 2.11.

cis-Adamantane-2-spiro-3′-8′-[4′-[2′-(4′-oxo-1′-piperidinyl)ethoxy]phenyl]-1′,2′,4′-trioxaspiro[4.5]decane mesylate (OZ520). A mixture of cis-adamantane-2-spiro-3′-8′-[4′-(2′-bromoethoxy)phenyl]-1′,2′,4′-trioxaspiro[4.5]decane (0.50 g, 1.08 mmol), 4-piperidone hydrochloride monohydrate (0.41 g, 2.67 mmol), and K₂CO₃ (2.0 g) in dry acetonitrile (80 ml) was heated at 60° C. for 3 d. After the reaction mixture was cooled to rt and filtered to remove the solid material, the filtrate was concentrated. The residue was washed with water (50 ml) and dried in vacuo. The crude product was dissolved in CH₂Cl₂ (5 ml) and then a solution of methanesulfonic acid (0.05 g, 0.52 mmol) in ethyl acetate (20 ml) was added. The precipitate was collected by filtration to afford trioxolane OZ520 (0.15 g, 24%) as a colorless solid. mp 156-157° C.; ¹H NMR (500 MHz, CDCl₃) δ 1.59-2.09 (m, 22H), 2.44-2.54 (m, 1H), 2.55-2.65 (m, 2H), 2.81 (brs, 3H), 3.23-3.36 (m, 4H), 3.61-3.69 (m, 2H), 4.01-4.11 (m, 2H), 4.47-4.53 (m, 2H), 6.84 (d, J=8.8 Hz, 2H), 7.15 (d, J=8.8 Hz, 2H), 12.24 (brs, 1H); ¹³C NMR (125.7 MHz, CDCl₃) δ 26.48, 26.87, 31.58, 34.66, 34.80, 36.41, 36.79, 37.76, 39.32, 42.03, 52.76, 56.05, 62.90, 108.30, 111.46, 114.45, 128.05, 140.23, 155.25, 200.54. Anal. Calcd for C₃₀H₄₃NO₈S: C, 62.37; H, 7.50; N, 2.42. Found: C, 62.26; H, 7.38; N, 2.32.

cis-Adamantane-2-spiro-3′-8′-[4′-[2′-(1′-piperazinyl)ethoxy]phenyl]-1′,2′,4′-trioxaspiro[4.5]decane dimesylate (OZ521). A mixture of cis-adamantane-2-spiro-3′-8′-[4′-(2′-bromoethoxy)phenyl]-1′,2′,4′-trioxaspiro[4.5]decane (0.50 g, 1.08 mmol), piperazine (0.25 g, 2.91 mmol), and K₂CO₃ (2.0 g) in dry acetonitrile (80 ml) was heated at 60° C. for 1 d. After the reaction mixture was cooled to rt and filtered to remove the solid material, the filtrate was concentrated. The residue was washed with water (50 ml) and dried in vacuo. The crude product was dissolved in CH₂Cl₂ (5 ml) and then a solution of methanesulfonic acid (0.20 g, 2.08 mmol) in ethyl acetate (30 ml) was added. The precipitate was collected by filtration to afford trioxolane OZ521 (0.51 g, 69%) as a colorless solid. mp 149-150° C.; ¹H NMR (500 MHz, CDCl₃) δ 1.59-2.09 (m, 22H), 2.42-2.53 (m, 1H), 2.72 (s, 6H), 3.59-3.96 (m, 10H), 4.36 (brs, 2H), 6.86 (d, J=8.8 Hz, 2H), 7.12 (d, J=8.8 Hz, 2H), 9.25 (brs, 2H), 10.08 (brs, 1H); ¹³C NMR (125.7 MHz, CDCl₃) δ 26.49, 26.89, 31.59, 34.66, 34.80, 36.41, 36.80, 39.49, 41.05, 41.98, 49.15, 56.33, 62.42, 108.30, 111.39, 114.72, 127.92, 140.02, 155.60. Anal. Calcd for C₃₀H₄₈N₂O₁₀S₂: C, 54.52; H, 7.32; N, 4.24. Found: C, 54.32; H, 7.21; N, 4.19.

cis-Adamantane-2-spiro-3′-8′-[4′-[2′-[4′-(methylsulfonyl)-1′-oxido-1′-piperidinyl]ethoxy]phenyl]-1′,2′,4′-trioxaspiro[4.5]decane mesylate (OZ522). Step 1. A mixture of cis-adamantane-2-spiro-3′-8′-[4′-(2′-bromoethoxy)phenyl]-1′,2′,4′-trioxaspiro[4.5]decane (0.50 g, 1.08 mmol), 4-methylthio-piperidine (0.18 g, 1.37 mmol), and K₂CO₃ (2.0 g) in dry acetonitrile (80 ml) was heated at 60° C. for 2 d. After the reaction mixture was cooled to rt and filtered to remove the solid material, the filtrate was concentrated. The residue was purified by crystallization from EtOH/H₂O (1:1) to afford the desired amine (0.30 g, 55%) as a colorless solid. ¹H NMR (500 MHz, CDCl₃) δ 1.52-2.07 (m, 26H), 2.09 (s, 3H), 2.16-2.25 (m, 2H), 2.44-2.60 (m, 2H), 2.78 (t, J=6.0 Hz, 2H), 2.93-3.02 (m, 2H), 4.07 (t, J=6.0 Hz, 2H), 6.83 (d, J=9.0 Hz, 2H), 7.10 (d, J=9.0 Hz, 2H); ¹³C NMR (125.7 MHz, CDCl₃) δ 13.03, 26.49, 26.89, 31.64, 32.21, 34.74, 34.80, 36.41, 36.81, 42.05, 42.37, 53.74, 57.31, 66.01, 108.43, 111.36, 114.45, 127.60, 138.48, 157.10. Step 2. To a solution of the above amine (0.30 g, 0.58 mmol) in CH₂Cl₂ (20 ml) at 0° C. was added dropwise a solution of m-CPBA (0.32 g, 1.27 mmol) in CH₂Cl₂ (10 ml). The resulting mixture was stirred at rt for 24 h and then partitioned between CH₂Cl₂ (20 ml) and saturated aq. NaHCO₃ (20 ml). The organic layer was washed with water (20 ml) and brine (20 ml), dried over MgSO₄, and filtered. After removal of the solvent, the crude product was purified by crystallization from EtOAc to afford trioxolane OZ522 (0.18 g, 55%) as a colorless solid. mp 150-151° C.; ¹H NMR (500 MHz, CDCl₃) δ 1.59-2.09 (m, 22H), 2.19 (d, J=12.7 Hz, 2H), 2.42-2.53 (m, 1H), 2.71-2.84 (m, 2H), 2.85-2.94 (m, 1H), 2.89 (s, 3H), 3.26 (t, J=11.7 Hz, 2H), 3.53 (d, J=11.2 Hz, 2H), 3.63 (brs, 2H), 4.61 (brs, 2H), 6.83 (d, J=8.8 Hz, 2H), 7.13 (d, J=8.3 Hz, 2H); ¹³C NMR (125.7 MHz, CDCl₃) δ 20.97, 26.49, 26.89, 31.61, 34.69, 34.81, 36.16, 36.41, 36.80, 42.04, 58.49, 61.36, 64.36, 70.78, 108.35, 111.43, 114.43, 127.90, 139.57, 155.81. Anal. Calcd for C₃₀H₄₃NO₇S: C, 64.14; H, 7.72; N, 2.49. Found: C, 58.32; H, 7.43; N, 2.28.

cis-Adamantane-2-spiro-3′-8′-[4′-[2′-(4′-formyl-1′-piperazinyl)ethoxy]phenyl]-1′,2′,4′-trioxaspiro[4.5]decane p-tosylate (OZ523). A mixture of cis-adamantane-2-spiro-3′-8′-[4′-(2′-bromoethoxy)phenyl]-1′,2′,4′-trioxaspiro[4.5]decane (0.50 g, 1.08 mmol), 1-piperazinecarboxaldehyde (0.30 g, 2.63 mmol), and K₂CO₃ (2.0 g) in dry acetonitrile (80 ml) was heated at 60° C. for 2 d. After the reaction mixture was cooled to rt and filtered to remove the solid material, the filtrate was concentrated. The residue was washed with water (50 ml) and dried in vacuo. The crude product was dissolved in CH₂Cl₂ (5 ml) and then a solution of p-toluenesulfonic acid monohydrate (0.17 g, 0.94 mmol) in ethyl acetate (30 ml) was added. The precipitate was collected by filtration to afford trioxolane OZ523 (0.42 g, 58%) as a colorless solid. mp 155-156° C.; ¹H NMR (500 MHz, CDCl₃) δ 1.59-2.09 (m, 22H), 2.37 (s, 3H), 2.44-2.55 (m, 1H), 2.84-2.99 (m, 2H), 3.35-3.46 (m, 1H), 3.54-3.64 (m, 2H), 3.70-4.00 (m, 4H), 4.39-4.46 (m, 2H), 4.53 (d, J=14.1 Hz, 1H), 6.76 (d, J=8.3 Hz, 2H), 7.12 (d, J=8.3 Hz, 2H), 7.19 (d, J=7.8 Hz, 2H), 7.76 (d, J=7.8 Hz, 2H), 8.06 (s, 1H), 12.15 (brs, 1H); ¹³C NMR (125.7 MHz, CDCl₃) δ 21.37, 26.48, 26.88, 31.59, 34.67, 34.81, 36.41, 36.57, 36.79, 42.03, 52.46, 53.27, 56.87, 62.79, 108.30, 111.46, 114.41, 125.83, 127.99, 129.01, 140.14, 140.57, 141.56, 155.19, 160.23. Anal. Calcd for C₃₆H₄₈N₂O₈S: C, 64.65; H, 7.23; N, 4.19. Found: C, 64.46; H, 7.22; N, 4.33.

cis-Adamantane-2-spiro-3′-8′-[4′-[3′-(1′H-imidazol-1′-yl)propoxy]phenyl]-1′,2′,4′-trioxaspiro[4.5]decane mesylate (OZ524). Step 1. To a solution of OZ288 (5.00 g, 14.04 mmol), 3-bromopropanol (2.93 g, 21.06 mmol), and triphenylphosphine (5.53 g, 21.06 mmol) in dry THF (75 ml) at 0° C. was added dropwise a solution of DIAD (4.26 g, 21.06 mmol) in THF (25 ml). After the reaction mixture was stirred at rt overnight, the solvent was removed under vacuum. The residue was dissolved in EtOAc (100 ml), washed with water (3×50 ml), dried over MgSO₄, and concentrated. The residue was purified by crystallization from EtOH (75 ml) to afford cis-adamantane-2-spiro-3′-8′-[4′-(3′-bromopropoxy)phenyl]-1′,2′,4′-trioxaspiro[4.5]decane (5.30 g, 79%) as a colorless solid. ¹H NMR (500 MHz, CDCl₃) δ 1.66 (m, 22H), 2.27-2.32 (m, 2H), 2.46-2.52 (m, 1H), 3.59 (t, J=6.4 Hz, 2H), 4.07 (t, J=5.9 Hz, 2H), 6.83 (d, J=8.8 Hz, 2H), 7.12 (d, J=8.8 Hz, 2H). Step 2. To a stirred mixture of cis-adamantane-2-spiro-3′-8′-[4′-(3′-bromopropoxy)phenyl]-1′,2′,4′-trioxaspiro[4.5]decane (0.60 g, 1.26 mmol) and potassium carbonate (2.0 g) in acetonitrile (50 ml) at rt was added imidazole (0.17 g, 2.52 mmol). The reaction mixture was stirred at 60° C. for 48 h and cooled to rt. The inorganic solid was filtered off and washed with EtOAc (2×25 ml). The combined filtrate was evaporated to dryness under vacuum. The residue was dissolved in EtOAc (50 ml), washed with water (3×25 ml), dried over MgSO₄, filtered. Removal of the solvent gave the desired free base as a colorless solid. To a solution of the above free base in EtOAc (10 ml) at 0° C. was added dropwise a solution of methanesulfonic acid (0.12 g, 1.26 mmol) in ether (10 ml). The resulting precipitate was filtered, washed with ether (3×10 ml), and dried under vacuum at 40° C. to afford trioxolane OZ524 (0.30 g, 43%) as a colorless solid. mp 149-151° C.; ¹H NMR (500 MHz, CDCl₃) δ 1.59-2.09 (m, 22H), 2.31-2.39 (m, 2H), 2.44-2.54 (m, 1H), 2.81 (s, 3H), 3.97 (t, J=5.6 Hz, 2H), 4.49 (t, J=7.2 Hz, 2H), 6.78 (d, J=8.3 Hz, 2H), 7.11 (d, J=8.8 Hz, 2H), 7.18 (s, 1H), 7.42 (s, 1H), 9.22 (s, 1H); ¹³C NMR (125.7 MHz, CDCl₃) δ 26.43, 26.83, 30.01, 31.56, 34.65, 34.75, 36.36, 36.74, 39.53, 41.94, 46.62, 63.64, 108.32, 111.35, 114.22, 120.42, 121.14, 127.76, 135.94, 139.11, 156.34. Anal. Calcd for C₂₉H₄₀N₂O₇S: C, 62.12; H, 7.19; N, 5.00. Found: C, 62.20; H, 7.16; N, 4.87.

cis-Adamantane-2-spiro-3′-8′-[4′-[3′-(4′-hydroxy-11′-piperidinyl)propoxy]phenyl]-1′,2′,4′-trioxaspiro[4.5]decane (OZ525). To a stirred mixture of cis-adamantane-2-spiro-3′-8′-[4′-(3′-bromopropoxy)phenyl]-1′,2′,4′-trioxaspiro[4.5]decane (0.40 g, 0.84 mmol) and potassium carbonate (1.20 g, 84 mmol) in acetonitrile (50 ml) at rt was added 4-hydroxypiperidine (0.17 g, 1.68 mmol). The reaction mixture was stirred at 60° C. for 48 h and cooled to rt. The inorganic solid was filtered off and washed with EtOAc (2×25 ml). The combined filtrate was evaporated to dryness under vacuum. The residue was dissolved in EtOAc (50 ml), washed with water (3×25 ml), dried over MgSO₄, and filtered. Removal of the solvent gave trioxolane OZ525 (0.41 g, 99%) as a colorless solid. mp 118-120° C.; ¹H NMR (500 MHz, CDCl₃) δ 1.52-2.09 (m, 29H), 2.10-2.22 (m, 2H), 2.42-2.55 (m, 3H), 2.72-2.84 (m, 2H), 3.64-3.74 (m, 1H), 3.97 (t, J=6.4 Hz, 2H), 6.82 (d, J=8.8 Hz, 2H), 7.10 (d, J=8.8 Hz, 2H); ¹³C NMR (125.7 MHz, CDCl₃) δ 26.45, 26.85, 27.06, 31.62, 34.45, 34.71, 34.76, 36.37, 36.77, 42.00, 51.13, 55.06, 66.24, 108.41, 111.31, 114.34, 127.53, 138.23, 157.30. Anal. Calcd for C₃₀H₄₃NO₅: C, 72.40; H, 8.71; N, 2.81. Found: C, 72.24; H, 8.96; N, 2.85.

cis-Adamantane-2-spiro-3′-8′-[4′-[3′-(3′,5′-dioxo-1′-piperazinyl)propoxy]phenyl]-1′,2′,4′-trioxaspiro[4.5]decane mesylate (OZ526). Step 1. To a stirred suspension of glycine methyl ester hydrochloride (5.00 g, 39.8 mmol) and potassium bicarbonate (9.96 g, 99.55 mmol) in acetonitrile (100 ml) at rt was added 2-bromoacetamide (5.5 g, 39.8 mmol). The reaction mixture was then refluxed for 8 h and cooled to rt. After filtration, the filtrate was concentrated. The solid residue was filtered, washed with chloroform (3×15 ml), acetone (3×15 ml), and dried to give piperazine-2,6-dione (0.85 g, 19%). ¹H NMR (500 MHz, DMSO-d₆) δ 3.34-3.38 (m, 4H), 10.81 (brs, 2H). Step 2. To a stirred mixture of cis-adamantane-2-spiro-3′-8′-[4′-(3′-bromopropoxy)phenyl]-1′,2′,4′-trioxaspiro[4.5]decane (0.60 g, 1.26 mmol) and potassium carbonate (2.00 g) in acetonitrile (50 ml) at rt was added piperazine-2,6-diones (0.29 g, 2.52 mmol). The reaction mixture was stirred for 48 h at 60° C. and cooled to rt. The inorganic solid was filtered off and washed with EtOAc (2×25 ml). The combined filtrate was evaporated to dryness under vacuum. The residue was dissolved in EtOAc (50 ml), washed with water (3×25 ml), dried over MgSO₄, and filtered. Removal of the solvent gave the desired free base as a colorless solid. To a solution of the above free base in EtOAc (10 ml) at 0° C. was added dropwise a solution of methanesulfonic acid (0.12 g, 1.26 mmol) in ether (10 ml). The resulting precipitate was filtered, washed with ether (3×10 ml), and dried under vacuum at 40° C. to afford trioxolane OZ526 (0.63 g, 82%) as a colorless solid. mp 149-151° C.; ¹H NMR (500 MHz, DMSO-d₆) δ 1.46-1.59 (m, 2H), 1.61-1.96 (m, 22H), 2.35 (s, 3H), 2.49-2.60 (m, 1H), 3.84 (t, J=6.8 Hz, 2H), 3.93 (t, J=6.1 Hz, 2H), 4.13 (s, 4H), 6.83 (d, J=8.3 Hz, 2H), 7.11 (d, J=8.3 Hz, 2H), 9.64 (brs, 2H); ¹³C NMR (125.7 MHz, DMSO-d₆) δ 25.99, 26.40, 27.33, 31.48, 34.29, 34.44, 35.96, 36.27, 36.35, 40.88, 45.12, 65.32, 108.32, 110.71, 114.58, 127.60, 138.19, 156.94, 165.81. Anal. Calcd for C₃₀H₄₂N₂O₉S: C, 59.39; H, 6.98; N, 4.62. Found: C, 59.60; H, 7.05; N, 4.40.

cis-Adamantane-2-spiro-3′-8′-[4′-[2′-[4′-(aminocarbonyl)-1′-piperazinyl]ethoxy]phenyl]-1′,2′,4′-trioxaspiro[4.5]decane p-tosylate (OZ528). A mixture of cis-adamantane-2-spiro-3′-8′-[4′-(2′-bromoethoxy)phenyl]-1′,2′,4′-trioxaspiro[4.5]decane (0.50 g, 1.08 mmol), piperazine-1-carboxamide hydrochloride (0.30 g, 1.81 mmol), and K₂CO₃ (2.0 g) in dry acetonitrile (80 ml) was heated at 60° C. for 2 d. After the reaction mixture was cooled to rt and filtered to remove the solid material, the filtrate was concentrated. The residue was washed with water (50 ml) and dried in vacuo. The crude product was dissolved in CH₂Cl₂ (5 ml) and then a solution of p-toluenesulfonic acid monohydrate (0.16 g, 0.89 mmol) in ethyl acetate (30 ml) was added. The precipitate was collected by filtration to afford trioxolane OZ528 (0.40 g, 54%) as a colorless solid. mp 153-154° C.; ¹H NMR (500 MHz, DMSO-d₆) δ 1.46-1.59 (m, 2H), 1.61-1.96 (m, 20H), 2.28 (s, 3H), 2.49-2.61 (m, 1H), 2.96-3.14 (m, 4H), 3.46-3.59 (m, 4H), 3.96-4.08 (m, 2H), 4.26-4.34 (m, 2H), 6.26 (s, 2H), 6.92 (d, J=8.3 Hz, 2H), 7.10 (d, J=7.8 Hz, 2H), 7.16 (d, J=8.8 Hz, 2H), 7.46 (d, J=7.8 Hz, 2H), 9.67 (brs, 1H); ¹³C NMR (125.7 MHz, DMSO-d₆) δ 20.98, 26.00, 26.41, 31.49, 34.27, 34.45, 35.98, 36.27, 40.75, 40.90, 51.53, 54.95, 62.03, 108.32, 110.77, 114.87, 125.68, 127.77, 128.24, 137.76, 139.15, 145.97, 156.02, 157.67. Anal. Calcd for C₃₆H₄₉N₃O₈S: C, 63.23; H, 7.22; N, 6.14. Found: C, 63.09; H, 6.99; N, 6.03.

cis-Adamantane-2-spiro-3′-8′-[4′-[2′-(3′-oxo-1′-piperazinyl)ethoxy]phenyl]-1′,2′,4′-trioxaspiro[4.5]decane p-tosylate (OZ529). A mixture of cis-adamantane-2-spiro-3′-8′-[4′-(2′-bromoethoxy)phenyl]-1′,2′,4′-trioxaspiro[4.5]decane (0.50 g, 1.08 mmol), 2-piperazinone (0.30 g, 3.0 mmol), and K₂CO₃ (2.0 g) in dry acetonitrile (80 ml) was heated at 60° C. for 2 d. After the reaction mixture was cooled to rt and filtered to remove the solid material, the filtrate was concentrated. The residue was washed with water (50 ml) and dried in vacuo. The crude product was dissolved in CH₂Cl₂ (5 ml) and then a solution of p-toluenesulfonic acid monohydrate (0.17 g, 0.94 mmol) in ethyl acetate (30 ml) was added. The precipitate was collected by filtration to afford trioxolane OZ529 (0.38 g, 54%) as a colorless solid. mp 156-157° C.; ¹H NMR (500 MHz, DMSO-d₆) δ 1.46-1.59 (m, 2H), 1.61-1.96 (m, 20H), 2.28 (s, 3H), 2.50-2.62 (m, 1H), 3.31-3.48 (m, 4H), 3.55-3.66 (m, 2H), 3.82-3.94 (m, 2H), 4.26-4.36 (m, 2H), 6.92 (d, J=8.3 Hz, 2H), 7.10 (d, J=7.8 Hz, 2H), 7.16 (d, J=8.3 Hz, 2H), 7.47 (d, J=8.3 Hz, 2H), 8.43 (brs, 1H), 10.15 (brs, 1H); ¹³C NMR (125.7 MHz, DMSO-d₆) δ 20.99, 26.01, 26.42, 31.50, 34.28, 34.46, 35.98, 36.28, 40.91, 47.98, 54.95, 108.33, 110.77, 114.86, 125.69, 127.78, 128.27, 137.85, 139.15, 145.86, 155.99. Anal. Calcd for C₃₅H₄₆N₂O₈S: C, 64.20; H, 7.08; N, 4.28. Found: C, 63.98; H, 7.12; N, 4.10.

cis-Adamantane-2-spiro-3′-8′-[4′-[2′-(3′,5′-dioxo-1′-piperazinyl)ethoxy]phenyl]-1′,2′,4′-trioxaspiro[4.5]decane p-tosylate (OZ530). A mixture of cis-adamantane-2-spiro-3′-8′-[4′-(2′-bromoethoxy)phenyl]-1′,2′,4′-trioxaspiro[4.5]decane (0.50 g, 1.08 mmol), 2,6-piperazinedione (0.25 g, 2.19 mmol), and K₂CO₃ (2.0 g) in dry acetonitrile (80 ml) was heated at 60° C. for 2 d. After the reaction mixture was cooled to rt and filtered to remove the solid material, the filtrate was concentrated. The crude product was dissolved in CH₂Cl₂ (5 ml) and then a solution of p-toluenesulfonic acid monohydrate (0.13 g, 0.72 mmol) in ethyl acetate (30 ml) was added. The precipitate was collected by filtration to afford trioxolane OZ530 (0.20 g, 28%) as a colorless solid. mp 149-151° C.; ¹H NMR (500 MHz, DMSO-d₆) δ 1.45-1.59 (m, 2H), 1.61-1.96 (m, 20H), 2.27 (s, 3H), 2.49-2.60 (m, 1H), 3.96-4.07 (m, 4H), 4.12 (s, 4H), 6.81 (d, J=8.8 Hz, 2H), 7.10 (d, J=7.8 Hz, 2H), 7.11 (d, J=7.8 Hz, 2H), 7.47 (d, J=8.3 Hz, 2H); ¹³C NMR (125.7 MHz, DMSO-d₆) δ 21.01, 26.03, 26.44, 31.52, 34.31, 34.48, 36.00, 36.30, 40.89, 45.29, 64.24, 108.36, 110.78, 114.55, 125.71, 127.72, 128.30, 137.92, 138.56, 145.79, 156.52, 166.08. Anal. Calcd for C₃₅H₄₄N₂O₉S: C, 62.86; H, 6.63; N, 4.19. Found: C, 63.00; H, 6.63; N, 4.03.

cis-Adamantane-2-spiro-3′-8′-[4′-[3′-[(2′-hydroxyethyl)ethylamino]propoxy]phenyl]-1′,2′,4′-trioxaspiro[4.5]decane mesylate (OZ531). To a stirred mixture of cis-adamantane-2-spiro-3′-8′-[4′-(3′-bromopropoxy)phenyl]-1′,2′,4′-trioxaspiro[4.5]decane (0.60 g, 1.26 mmol) and potassium carbonate (2.00 g) in acetonitrile (50 ml) at rt was added 2-(ethylamino)ethanol (0.23 g, 2.52 mmol). The reaction mixture was stirred for 48 h at 60° C. and cooled to rt. The inorganic solid was filtered off and washed with EtOAc (2×25 ml). The combined filtrate was evaporated to dryness under vacuum. The residue was dissolved in EtOAc (50 ml), washed with water (3×25 ml), dried over MgSO₄, filtered. Removal of the solvent gave the desired free base as a colorless solid. To a solution of the above free base in EtOAc (10 ml) at 0° C. was added dropwise a solution of methanesulfonic acid (0.12 g, 1.26 mmol) in ether (10 ml). The resulting precipitate was filtered, washed with ether (3×10 ml), and dried under vacuum at 40° C. to afford trioxolane OZ531 (0.60 g, 82%) as a colorless solid. mp 135-137° C.; ¹H NMR (500 MHz, CDCl₃) δ 1.39 (t, J=7.6 Hz, 3H), 1.61-2.09 (m, 22H), 2.21-2.35 (m, 2H), 2.46-2.53 (m, 1H), 2.75 (s, 3H), 3.23-3.46 (m, 6H), 3.99 (t, J=4.8 Hz, 2H), 4.05 (t, J=5.4 Hz, 2H), 4.06-4.19 (m, 1H), 6.80 (d, J=8.8 Hz, 2H), 7.11 (d, J=8.8 Hz, 2H), 9.72 (brs, 1H); ¹³C NMR (125.7 MHz, CDCl₃) δ 8.41, 23.75, 26.39, 26.79, 31.53, 34.61, 34.71, 36.32, 36.71, 39.24, 41.90, 48.76, 50.89, 55.29, 56.18, 64.57, 108.28, 111.30, 114.24, 127.66, 139.01, 156.44. Anal. Calcd for C₃₀H₄₇NO₈S: C, 61.94; H, 8.14; N, 2.41. Found: C, 62.18; H, 8.17; N, 2.07.

cis-Adamantane-2-spiro-3′-8′-[4′-[3′-[bis(2′-hydroxyethyl)amino]propoxy]phenyl]-1′,2′,4′-trioxaspiro[4.5]decane p-tosylate (OZ532). To a stirred mixture of cis-adamantane-2-spiro-3′-8′-[4′-(3′-bromopropoxy)phenyl]-1′,2′,4′-trioxaspiro[4.5]decane (0.60 g, 1.26 mmol) and potassium carbonate (2.0 g) in acetonitrile (50 ml) at rt was added diethanolamine (0.27 g, 2.52 mmol). The reaction mixture was stirred for 48 h at 60° C. and cooled to rt. The inorganic solid was filtered off and washed with EtOAc (2×25 ml). The combined filtrate was evaporated to dryness under vacuum. The residue was dissolved in EtOAc (50 ml), washed with water (3×25 ml), dried over MgSO₄, and filtered. Removal of the solvent gave the desired free base as a colorless solid. To a solution of the above free base in EtOAc (10 ml) at 0° C. was added dropwise a solution of p-toluenesulfonic acid monohydrate (0.24 g, 1.26 mmol) in ether (10 ml). The resulting precipitate was filtered, washed with ether (3×10 ml), and dried under vacuum at 40° C. to afford trioxolane OZ532 (0.60 g, 69%) as a colorless solid. mp 126-128° C.; ¹H NMR (500 MHz, DMSO-d₆) δ 1.45-1.59 (m, 2H), 1.61-1.96 (m, 20H), 2.08-2.16 (m, 2H), 2.29 (s, 3H), 2.48-2.60 (m, 1H), 3.19-3.41 (m, 6H), 3.71-3.81 (m, 4H), 4.01 (t, J=6.2 Hz, 2H), 5.29 (t, J=4.4 Hz, 2H), 6.86 (d, J=8.3 Hz, 2H), 7.11 (d, J=7.8 Hz, 2H), 7.13 (d, J=8.8 Hz, 2H), 7.47 (d, J=8.3 Hz, 2H), 9.03 (brs, 1H); ¹³C NMR (125.7 MHz, DMSO-d₆) δ 20.95, 23.36, 26.00, 26.41, 31.48, 34.28, 34.44, 35.97, 36.27, 40.87, 50.89, 54.80, 55.38, 65.01, 108.31, 110.72, 114.58, 125.67, 127.64, 128.25, 137.84, 138.44, 145.82, 156.70. Anal. Calcd for C₃₆H₅₁NO₉S: C, 64.17; H, 7.63; N, 2.08. Found: C, 64.17; H, 7.54; N, 2.09.

cis-Adamantane-2-spiro-3′-8′-[4′-[3′-(3′-oxo-1′-piperazinyl)propoxy]phenyl]-1′,2′,4′-trioxaspiro[4.5]decane mesylate (OZ533). To a stirred mixture of cis-adamantane-2-spiro-3′-8′-[4′-(3′-bromopropoxy)phenyl]-1′,2′,4′-trioxaspiro[4.5]decane (0.60 g, 1.26 mmol) and potassium carbonate (2.00 g) in acetonitrile (50 ml) at rt was added 2-oxopiperazine (0.25 g, 2.52 mmol). The reaction mixture was stirred for 48 h at 60° C. and cooled to rt. The inorganic solid was filtered off and washed with EtOAc (2×25 ml). The combined filtrate was evaporated to dryness under vacuum. The residue was dissolved in EtOAc (50 ml), washed with water (3×25 ml), dried over MgSO₄, and filtered. Removal of the solvent gave the desired free base as a colorless solid. To a solution of the above free base in EtOAc (10 ml) at 0° C. was added dropwise a solution of methanesulfonic acid (0.12 g, 1.26 mmol) in ether (10 ml). The resulting precipitate was filtered, washed with ether (3×10 ml), and dried under vacuum at 40° C. to afford trioxolane OZ533 (0.35 g, 47%) as a colorless solid. mp 156-158° C.; ¹H NMR (500 MHz, DMSO-d₆) δ 1.45-1.59 (m, 2H), 1.61-1.96 (m, 20H), 2.08-2.19 (m, 2H), 2.35 (s, 3H), 2.48-2.60 (m, 1H), 3.23-3.51 (m, 4H), 3.61-3.99 (m, 4H), 4.02 (t, J=5.8 Hz, 2H), 6.87 (d, J=8.8 Hz, 2H), 7.14 (d, J=8.3 Hz, 2H), 8.45 (s, 1H), 10.05 (brs, 1H); ¹³C NMR (125.7 MHz, DMSO-d₆) δ 23.64, 25.99, 26.40, 31.48, 34.27, 34.43, 35.96, 36.26, 37.04, 40.87, 47.59, 52.42, 53.43, 64.80, 108.30, 110.71, 114.59, 127.65, 138.48, 156.66, 162.59. Anal. Calcd for C₃₀H₄₄N₂O₈S: C, 60.79; H, 7.48; N, 4.73. Found: C, 60.61; H, 7.48; N, 4.62.

cis-Adamantane-2-spiro-3′-8′-[4′-[3′-[4′-(aminocarbonyl)-1′-piperidinyl]propoxy]phenyl]-1′,2′,4′-trioxaspiro[4.5]decane p-tosylate (OZ536). To a stirred mixture of cis-adamantane-2-spiro-3′-8′-[4′-(3′-bromopropoxy)phenyl]-1′,2′,4′-trioxaspiro[4.5]decane (0.60 g, 1.26 mmol) and potassium carbonate (2.00 g) in acetonitrile (50 ml) at rt was added isonipecotamide (0.32 g, 2.52 mmol). The reaction mixture was stirred for 48 h at 60° C. and cooled to rt. The inorganic solid was filtered off and washed with EtOAc (2×25 ml). The combined filtrate was evaporated to dryness under vacuum. The residue was dissolved in EtOAc (50 ml), washed with water (3×25 ml), dried over MgSO₄, and filtered. Removal of the solvent gave the desired free base as a colorless solid. To the solution of the above free base in EtOAc (10 ml) at 0° C. was added dropwise a solution of p-toluenesulfonic acid monohydrate (0.07 g, 0.36 mmol) in ether (10 ml). The resulting precipitate was filtered, washed with ether (3×10 ml), and dried under vacuum at 40° C. to afford trioxolane OZ536 (0.25 g, 29%) as a colorless solid. mp 150-152° C.; ¹H NMR (500 MHz, DMSO-d₆) δ 1.45-1.59 (m, 2H), 1.61-1.96 (m, 24H), 2.02-2.17 (m, 2H), 2.29 (s, 3H), 2.30-2.41 (m, 1H), 2.47-2.60 (m, 1H), 2.86-2.99 (m, 2H), 3.16-3.25 (m, 2H), 3.55 (d, J=11.7 Hz, 2H), 4.00 (t, J=5.6 Hz, 2H), 6.85 (d, J=8.8 Hz, 2H), 6.95 (s, 1H), 7.12 (d, J=7.8 Hz, 2H), 7.13 (d, J=8.8 Hz, 2H), 7.42 (s, 1H), 7.48 (d, J=7.8 Hz, 2H), 8.96 (brs, 1H); ¹³C NMR (125.7 MHz, DMSO-d₆) δ 20.98, 23.78, 26.01, 26.10, 26.42, 31.51, 34.29, 34.46, 35.98, 36.28, 38.80, 40.89, 51.52, 53.79, 64.92, 108.33, 110.74, 114.57, 125.69, 127.68, 128.30, 137.91, 138.48, 145.77, 156.69, 174.91. Anal. Calcd for C₃₈H₅₂N₂O₈S: C, 65.49; H, 7.52; N, 4.02. Found: C, 65.28; H, 7.75; N, 4.11.

cis-Adamantane-2-spiro-3′-8′-[4′-[2′-[(2′-amino-2′-oxoethyl)methylamino]ethoxy]phenyl]-1′,2′,4′-trioxaspiro[4.5]decane mesylate (OZ538). A mixture of cis-adamantane-2-spiro-3′-8′-[4′-(2′-bromoethoxy)phenyl]-1′,2′,4′-trioxaspiro[4.5]decane (0.50 g, 1.08 mmol), sarcosine amide hydrochloride (0.27 g, 2.17 mmol), and K₂CO₃ (2.0 g) in dry acetonitrile (80 ml) was heated at 60° C. for 3 d. After the reaction mixture was cooled to rt and filtered to remove the solid material, the filtrate was concentrated. The crude product was dissolved in CH₂Cl₂ (5 ml) and then a solution of methanesulfonic acid (0.06 g, 0.63 mmol) in ethyl acetate (20 ml) was added. The precipitate was collected by filtration to afford trioxolane OZ538 (0.36 g, 59%) as a colorless solid. mp 149-150° C.; ¹H NMR (500 MHz, CDCl₃) δ 1.61-2.09 (m, 22H), 2.46-2.53 (m, 1H), 2.68 (s, 3H), 3.07 (s, 3H), 3.59-3.84 (m, 2H), 4.09-4.43 (m, 4H), 6.83 (d, J=8.8 Hz, 2H), 6.84 (s, 1H), 7.09 (d, J=8.3 Hz, 2H), 8.01 (s, 1H), 9.77 (brs, 1H); ¹³C NMR (125.7 MHz, CDCl₃) δ 26.49, 26.89, 31.60, 34.67, 34.80, 36.41, 36.81, 39.40, 41.98, 42.38, 55.62, 57.07, 62.71, 108.32, 111.36, 114.59, 127.86, 139.73, 155.72, 166.72. Anal. Calcd for C₂₈H₄₂N₂O₈S: C, 59.34; H, 7.47; N, 4.94. Found: C, 59.10; H, 7.75; N, 4.79.

### Example 4

### Treatment of Fascioliasis Using Trioxolanes

### Materials and Methods

OZ72, OZ78, OZ352, and OZ418 were prepared as a suspension in 7% (v/v) Tween-80 and 3% (v/v) ethanol before oral administration. Metacercariae of F. hepatica were purchased from G. Graham (Addlestone, UK). Female Wistar rats (n=32, age: 5 weeks, weight: ˜100 g) were purchased from RCC (Itingen, Switzerland). Animals were kept in groups of 5 in macrolon cages in environmentally-controlled conditions (temperature: ˜25° C.; humidity: ˜70%; 12 h light/dark cycle) and acclimatized for 1 week. They had free access to water and rodent diet. Thirty-two rats were infected intragastrically with 25 metacercarial cysts of F. hepatica each. Eight to 9 weeks post-infection, 4 groups of 5 rats were treated orally with OZ78 at single doses ranging from 50 to 400 mg/kg. Twelve untreated rats served as control group. Ten days post-treatment, rats were euthanised by CO₂. At necropsy F. hepatica were harvested from the excised bile ducts and counted. Average worm burdens were expressed as arithmetic means, including values of zero for animals with no worms. The Kruskal-Wallis (KW) test was used to compare the median of the responses among the treatment groups. A difference in median was considered to be significant at a level of 5%. Statistical analyses were done with Version 2.4.5 of Statsdirect Statistical software (Statsdirect LtD; Cheshire, UK).

The effects of the four trioxolanes (OZ) on adult F. hepatica harbored in rats are summarized in Table 4. Trioxolanes that are the subject of the inventors' previous patent application, OZ78 and OZ418 had good curative efficacies, whereas OZ72 and OZ352 were only weakly active against F. hepatica. For OZ78, administration of single oral doses of 100 mg/kg and above resulted in worm burden reductions of 100%. Even at the lowest dose (50 mg/kg) investigated, a statistically significant worm burden reduction of 53% was obtained (KW=4.53; P=0.033). For comparison, a slightly lower dose of the drug of choice for fascioliasis, triclabendazole, namely 40 mg/kg resulted in worm burden reductions of 99% in F. hepatica-infected rats.

Earlier work on the effect of artesunate on F. hepatica is also summarized in Table 4 [Keiser et al., 2006]. Administration of artesunate at a single oral dose of 400 mg/kg to F. hepatica-infected rats resulted in worm burden reductions of 100%. At half this dose, a worm burden reduction of 71.4% was achieved. The lowest oral dose investigated, 100 mg/kg was not effective, yielding only a low worm burden reduction of 30%. The higher doses of artesunate were toxic; at the 200 mg/kg dose, one rat died following treatment, and at the 400 mg/kg dose, three rats died following treatment. This compares to the relative safety of OZ78 where no mortality was observed at doses up to 1000 mg/kg. The effect OZ78 on C. sinensis harbored in rats was as follows. A single dose of 300 mg/kg OZ78 resulted in worm burden reductions of 78.5% (1/4 cures) and 98.5% (4/5 cures) against juvenile and adult C. sinensis, respectively. A single dose of 150 mg/kg OZ78 resulted in worm burden reductions of 46.0% (0/4 cures) and 85.3% (1/4 cures) against juvenile and adult C. sinensis, respectively. For comparison, a single oral dose of 375 mg/kg praziquantel, the current drug of choice for clonorchiasis, has been found to be the minimum effective dose in rats [Fan, 2005]. At a lower dose of 275 mg/kg, praziquantel achieved only a moderate worm burden reduction of 33% [Fan, 2005].

In summary, selected trioxolanes (OZ) were found to be highly efficacious against two liver flukes Fasciola hepatica and Clonorchis sinensis.

TABLE 4

| | | | Total worm |
|---|---|---|---|
| | | No. of rats | burden |
| Treatment | Dose (mg/kg) | cured | reduction (%) |
| Control | — | 0/12 | — |
| OZ72 | 100 | 1/4 | 52 |
| OZ78 | 50 | 0/5 | 53 |
| | 100 | 5/5 | 100 |
| | 200 | 5/5 | 100 |
| | 400 | 5/5 | 100 |
| OZ352 | 100 | 0/5 | 14 |
| OZ418 | 50 | 0/5 | 23 |
| | 100 | 4/5 | 99 |
| Artesunate | 100 | 2/5 | 30 |
| | 200^{a} | 1/5 | 71 |
| | 400^{b} | 2/5 | 100 |

^{a}1 rat died following treatment
^{b}3 rats died following treatment

It should be appreciated that the spiro and dispiro 1,2,4-trioxolane compositions of this invention may contain trioxolanes within the scope of the formulas described above, or prodrugs or analogues of these compounds or a racemic mixture of either the D or the L form. The invention is also intended to include all biologically active salt forms of the compounds. Also, minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

What is claimed is:

1. A spiro or dispiro 1,2,4-trioxolane selected from the group consisting of OZ406, OZ407, OZ408, OZ409, OZ410, OZ411, OZ412, OZ413, OZ414, OZ415, OZ416, OZ417, OZ418, OZ419, OZ420, OZ421, OZ422, OZ423, OZ424, OZ425, OZ426, OZ427, OZ428, OZ430, OZ431, OZ432, OZ433, OZ434, OZ435, OZ436, OZ437, OZ438, OZ440, OZ441, OZ442, OZ443, OZ444, OZ445, OZ446, OZ447, OZ448, OZ449, OZ450, OZ451, OZ452, OZ454, OZ455, OZ456, OZ457, OZ458, OZ459, OZ463, OZ465, OZ467, OZ468, OZ469, OZ470, OZ471, OZ472, OZ473, OZ474, OZ475, OZ476, OZ477, OZ478, OZ479, OZ480, OZ481, OZ483, OZ484, OZ486, OZ487, OZ488, OZ489, OZ490, OZ491, OZ492, OZ494, OZ496, OZ497, OZ500, OZ501, OZ502, OZ503, OZ504, OZ506, OZ507, OZ509, OZ510, OZ511, OZ512, OZ514, OZ515, OZ516, OZ517, OZ518, OZ519, OZ520, OZ521, OZ522, OZ523, OZ524, OZ525, OZ526, OZ528, OZ529, OZ530, OZ531, OZ532, OZ533, OZ536, OZ538, and their prodrugs and analogues.

2. The spiro or dispiro 1,2,4-trioxolane of claim 1 that is selected from the group consisting of OZ413, OZ433, OZ434, OZ435, OZ436, OZ457, OZ465, OZ480, OZ481, OZ494, OZ501, and OZ511.

3. The spiro or dispiro 1,2,4-trioxolane of claim 1 further including a pharmaceutically acceptable carrier.

4. A method of preventing or treating malaria comprising:
administrating a malaria prevention or malaria treatment effective amount of a spiro or dispiro 1,2,4-trioxolane in a pharmaceutically acceptable carrier, said trioxolane being selected from the group consisting of OZ406, OZ407, OZ408, OZ409, OZ410, OZ411, OZ412, OZ413, OZ414, OZ415, OZ416, OZ417, OZ418, OZ419, OZ420, OZ421, OZ422, OZ423, OZ424, OZ425, OZ426, OZ427, OZ428, OZ430, OZ431, OZ432, OZ433, OZ434, OZ435, OZ436, OZ437, OZ438, OZ440, OZ441, OZ442, OZ443, OZ444, OZ445, OZ446, OZ447, OZ448, OZ449, OZ450, OZ451, OZ452, OZ454, OZ455, OZ456, OZ457, OZ458, OZ459, OZ463, OZ465, OZ467, OZ468, OZ469, OZ470, OZ471, OZ472, OZ473, OZ474, OZ475, OZ476, OZ477, OZ478, OZ479, OZ480, OZ481, OZ483, OZ484, OZ486, OZ487, OZ488, OZ489, OZ490, OZ491, OZ492, OZ494, OZ496, OZ497, OZ500, OZ501, OZ502, OZ503, OZ504, OZ506, OZ507, OZ509, OZ510, OZ511, OZ512, OZ514, OZ515, OZ516, OZ517, OZ518, OZ519, OZ520, OZ521, OZ522, OZ523, OZ524, OZ525, OZ526, OZ528, OZ529, OZ530, OZ531, OZ532, OZ533, OZ536, OZ538, and combinations of the same.

5. A method of manufacturing a composition for prophylaxis and treatment of malaria comprising: mixing at least one malaria prophylaxis or malaria treatment-effective amount of a spiro or dispiro 1,2,4-trioxolane, its prodrugs and optical isomers thereof, with a pharmaceutically acceptable carrier, said trioxolane being selected from the group consisting of: OZ406, OZ407, OZ408, OZ409, OZ410, OZ411, OZ412, OZ413, OZ414, OZ415, OZ416, OZ417, OZ418, OZ419, OZ420, OZ421, OZ422, OZ423, OZ424, OZ425, OZ426, OZ427, OZ428, OZ430, OZ431, OZ432, OZ433, OZ434, OZ435, OZ436, OZ437, OZ438, OZ440, OZ441, OZ442, OZ443, OZ444, OZ445, OZ446, OZ447, OZ448, OZ449, OZ450, OZ451, OZ452, OZ454, OZ455, OZ456, OZ457, OZ458, OZ459, OZ463, OZ465, OZ467, OZ468, OZ469, OZ470, OZ471, OZ472, OZ473, OZ474, OZ475, OZ476, OZ477, OZ478, OZ479, OZ480, OZ481, OZ483, OZ484, OZ486, OZ487, OZ488, OZ489, OZ490, OZ491, OZ492, OZ494, OZ496, OZ497, OZ500, OZ501, OZ502, OZ503, OZ504, OZ506, OZ507, OZ509, OZ510, OZ511, OZ512, OZ514, OZ515, OZ516, OZ517, OZ518, OZ519, OZ520, OZ521, OZ522, OZ523, OZ524, OZ525, OZ526, OZ528, OZ529, OZ530, OZ531, OZ532, OZ533, OZ536, OZ538, and their prodrugs and analogues.

6. A method of prophylaxis or treatment of schistosomiasis comprising:
administrating a schistosomiasis prophylaxis or treatment effective amount of a spiro or dispiro 1,2,4-trioxolane in a pharmaceutically acceptable carrier, said trioxolane being selected from the group consisting of: OZ406, OZ407, OZ408, OZ409, OZ410, OZ411, OZ412, OZ413, OZ414, OZ415, OZ416, OZ417, OZ418, OZ419, OZ420, OZ421, OZ422, OZ423, OZ424, OZ425, OZ426, OZ427, OZ428, OZ430, OZ431, OZ432, OZ433, OZ434, OZ435, OZ436, OZ437, OZ438, OZ440, OZ441, OZ442, OZ443, OZ444, OZ445, OZ446, OZ447, OZ448, OZ449, OZ450, OZ451, OZ452, OZ454, OZ455, OZ456, OZ457, OZ458, OZ459, OZ463, OZ465, OZ467, OZ468, OZ469, OZ470, OZ471, OZ472, OZ473, OZ474, OZ475, OZ476, OZ477, OZ478, OZ479, OZ480, OZ481, OZ483, OZ484, OZ486, OZ487, OZ488, OZ489, OZ490, OZ491, OZ492, OZ494, OZ496, OZ497, OZ500, OZ501, OZ502, OZ503, OZ504, OZ506, OZ507, OZ509, OZ510, OZ511, OZ512, OZ514, OZ515, OZ516, OZ517, OZ518, OZ519, OZ520, OZ521, OZ522, OZ523, OZ524, OZ525, OZ526, OZ528, OZ529, OZ530, OZ531, OZ532, OZ533, OZ536, OZ538, and their prodrugs and analogues.

7. A method of prophylaxis or treatment of fascioliasis comprising: administrating a fascioliasis prophylaxis or treatment effective amount of a spiro or dispiro 1,2,4-trioxolane in a pharmaceutically acceptable carrier, said trioxolane having the following structure: 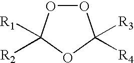
wherein R₁, R₂, R₃, and R₄ are the same or different, and are selected from the group consisting of substituted or unsubstituted linear or branched alkyl, aryl, and alkaryl groups and substituted or unsubstituted alicyclic groups that may be interrupted by one or more oxygen, sulfur or nitrogen atoms, and substituted or unsubstituted aromatic or heterocyclic groups, whereby none of R₁, R₂, R₃, or R₄ may be hydrogen; and further providing that R₁ and R₂ taken together and/or R₃ and R₄ taken together may form a substituted or unsubstituted alicyclic group which is optionally interrupted by one or more oxygen, sulfur or nitrogen atoms.

8. The method of claim 7 whereby the trioxolane is selected from the group consisting of OZ78, OZ288, OZ418, and combinations of the same

9. The method of claim 7 whereby the trioxolane is administered in a dose ranging from about 100-200 mg/kg/day.

10. The method of claim 7 whereby the trioxolane is administered orally.
